SEQUENCE LISTING (11 sequences)

SEQ ID NO: 1 - protein, 8 aa, Artificial Sequence
  Synthetic Construct
SIINFEKL

SEQ ID NO: 2 - DNA, 19 bp, Artificial Sequence
  Synthetic Construct
acggctaccgtctggacat

SEQ ID NO: 3 - DNA, 18 bp, Artificial Sequence
  Synthetic Construct
cgatggcttcttcgatgc

SEQ ID NO: 4 - DNA, 28 bp, Artificial Sequence
  Synthetic Construct
aaccaagctttcggcgattgtgatgagg

SEQ ID NO: 5 - DNA, 32 bp, Artificial Sequence
  Synthetic Construct
aaccaagcttctatccgggctatacccaagac

SEQ ID NO: 6 - DNA, 30 bp, Artificial Sequence
  Synthetic Construct
aaccaagctttgtttgcgctggacagtccc

SEQ ID NO: 7 - DNA, 30 bp, Artificial Sequence
  Synthetic Construct
aaccaagcttaacgcgtcctcccttgatgg

SEQ ID NO: 8 - DNA, 21 bp, Artificial Sequence
  Synthetic Construct
gatgccatcaatggccaggag

SEQ ID NO: 9 - DNA, 19 bp, Artificial Sequence
  Synthetic Construct
acgaaagtcgcgcaggaag

SEQ ID NO: 10 - DNA, 20 bp, Artificial Sequence
  Synthetic Construct
gttgaccacgatgccgatcc

SEQ ID NO: 11 - DNA, 20 bp, Artificial Sequence
  Synthetic Construct
gcatgacgaaggcgtagctc

(19) United States
(12) Patent Application Publication
(10) Pub. No.: US 2014/0363465 A1
(43) Pub. Date: Dec. 11, 2014

(54) MYCOBACTERIAL VACCINE VECTORS AND METHODS OF USING THE SAME

(71) Applicants: Norman L Letvin, Newton, MA (US); Michael W. Panas, Westborough, MA (US); Geoff Gillard, Newton, MA (US)

(72) Inventors: Norman L Letvin, Newton, MA (US); Michael W. Panas, Westborough, MA (US); Geoff Gillard, Newton, MA (US)

(73) Assignee: Beth Israel Deaconess Medical Center, Inc., Boston, MA (US)

(21) Appl. No.: 14/009,376

(22) PCT Filed: Apr. 4, 2012
(86) PCT No.: PCT/US12/32164

§ 371 Date: Aug. 27, 2014

Related U.S. Application Data
(60) Provisional application No. 61/471,516, filed on Apr. 4, 2011.

(51) Int. Cl. A61K 39/00; A61K 39/12; A61K 39/02

(52) U.S. Cl. CPC A61K 39/0002; A61K 39/0003; A61K 39/02; A61K 39/12; A61K 2039/70
USPC 424/200.1; 435/252.3; 435/6.15

(57) ABSTRACT

Mycobacterium bovis BCG is a potent stimulator of the cellular immune response and has potential as a recombinant vaccine vector. Disclosed are BCG strains that generate greater MHC class I presentation of a transgenic protein, relative to the unmutated parental strain, and that improve a recipient's CD8+ T cell response against the transgenic protein following vaccination. The mycobacterial constructs and their respective t/ansposon mutant BCG strains exhibit increased immunogenicity that is several times greater than responses generated by the parental strain and other existing modified rBCG strains. Furthermore, upon introducing the SIV gag gene into these novel strains, we observed that these strains primed for increased CD8+ T cell responses in a heterologous prime/boost regimen, comparable to levels generated by plasmid DNA vaccines. Creation of a second generation rBCG vector that may be utilized as a vaccine vector for immunizing against a variety of pathogens.

### FIELD OF THE INVENTION

The invention provides mycobacterial vectors with increased immunogenicity and methods of using the vectors as vaccines. The vectors can include any of a number of foreign antigens (e.g., pathogen, cancer, or allergen-based antigens) for use as an immunizing agent.

### BACKGROUND OF THE INVENTION

Vaccines have had a profound impact on the control of infectious disease. The development of live recombinant vectors in the past three decades has greatly advanced the field of vaccinology and immunology. Novel live recombinant vectors will most likely be a source of future vaccines for emerging infectious agents and complex pathogens for which traditional vaccines have provided inadequate protection.

Since the initial creation of a live recombinant pox virus delivering influenza hemagglutinin, recombinant vaccine vectors have been created out of a diversity of viruses and bacteria [1]. Key features of recombinant vectors are in vivo replication, induction of a memory immune response, a genome capable of tolerating large foreign genes, and expression of those foreign genes in a form that is nearly identical to that expressed under natural conditions [2]. In addition, a successful recombinant vector must be cost effective, have a good safety profile, have high and durable expression of the transgenic protein, and have the ability to induce a robust transgene product-specific immune response at the site of infection of the pathogen [3].

Each specific live vector stimulates the immune response in a unique fashion based upon its life cycle in the host, the pathogen associated molecular patterns (PAMPS) it contains, and the limited pathology it causes. Some vectors induce a stronger humoral immune response, some vectors induce a stronger cellular immune response, and some vectors induce both types of responses. Each pathogen is controlled by the immune response in a particular way, so it is necessary to select a vector for creating a vaccine that induces an immune response appropriate for the control and clearance of the specific pathogen [4]. Also, as there is often a correlation between pathogenicity of a vector and its immunogenicity, it is important to balance potent immunogenicity and a tolerable safety profile.

One live vector, recombinant Mycobacterium bovis BCG (rBCG), stands out as a potential vaccine vector for the stimulation of cellular immune responses. The original application of BCG was to combat tuberculosis. However, through the expression of foreign transgenic proteins, there are a large number of infectious agents in humans for which one could use rBCG. rBCG is a strong T cell-inducting vaccine vector because of its unique biological properties; specifically, it contains many highly immunogenic lipoproteins and is an intracellular pathogen capable of surviving in macrophages and continuously synthesizing proteins. Therefore, rBCG is an attractive delivery vector because it is highly immunogenic and can persist within the host where it replicates and produces a transgene product. The safety profile of BCG has been borne out by the fact that it has been given to approximately 3 billion individuals with minimal adverse effects [2]. In addition, rBCG has the potential to stimulate a large cellular immune response skewed to a Th1 phenotype that is targeted to the mucosal area [5, 6]. Furthermore, when combined with a protein boost, rBCG vaccines have demonstrated the ability to enhance transgene protein-specific antibody responses [7, 8].

The ability to generate robust cytotoxic T lymphocyte (CTL) and T helper cell responses in combination with antibody responses, all targeted to mucosal surfaces, suggest that rBCG has potential as an effective vaccine vector. However, as a derivative of the pathogenic M. bovis strain, rBCG contains genes that limit the effective immune response against a transgenic protein. First generation recombinant BCG vaccines have been created and tested in simian models and human clinical trials with some success [9-12]. The robust BCG vector-specific responses suggest the potential of this vaccine, although responses of the same magnitude against a transgenic protein have not been realized. There is good reason to believe that the limitations that exist in using rBCG as a vector vaccine reflect the biology of BCG, and that elimination of these limitations would substantially increase the potential of BCG as a vaccine vector. There is need to engineer BCG so as to address these limitations and harness BCG as a vaccine vector.

### SUMMARY OF THE INVENTION

In a first aspect, the invention features a mycobacterium that includes one or more mutations (e.g., one or more deletions, substitutions, or insertions) that ablate or reduce expression of at least one (e.g., two, three, four, or more) gene selected from BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and BCG_{—}3808c, or a homolog thereof, or an operon that includes the gene. In an embodiment, the mutation is a deletion of all or a part of the gene(s), an operon(s) including the gene(s), or a promoter region(s) for the gene(s). In another embodiment, the mycobacterium is selected from M. africanum, M. avium, M. bovis, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi (e.g., the mycobacterium is M. bovis BCG). In another embodiment, the mycobacterium is engineered to express a foreign antigen, epitope, or polypeptide (e.g., a foreign antigen, epitope, or polypeptide from a pathogen (e.g., a virus (e.g., HIV, such as gp120 env, gp140 env, gp160 env, gag, pol, vif, vpr, vpu, tat, rev or nef), bacteria, fungus, or parasite), a cancer cell, and/or an allergen, and/or a foreign antigen, epitope, or polypeptide associated with an autoimmune disease or graft rejection, and/or a foreign antigen, epitope, or polypeptide selected from a cytokine, a chemokine, an immunoregulatory agent, and/or a therapeutic agent). In other embodiments, a nucleic acid molecule encoding the epitope, antigen, or polypeptide is incorporated within the genome of the mycobacterium, incorporated within a nucleic acid vector (e.g., a plasmid) that is stably transformed in the mycobacterium; and/or incorporated at the site of the at least one gene (e.g., at the site of one or more of the following genes: BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c, or a homolog thereof, or at the site of an operon that includes the gene(s)).

In other embodiments of the first aspect of the invention, the mycobacterium is a vector (e.g., a vaccine vector). In other embodiments, the mycobacterium is nonpathogenic (e.g., nonpathogenic to a subject (e.g., a human) to which the mycobacterium is administered). In other embodiments, the mycobacterium is clone G9, J13, A79, AK27, C46, K14, BC15, CN11, A25, AF30, C63, AF25, C57, BL2, AE29, AZ11, or CX18.

In still other embodiments of the first aspect of the invention, the mycobacterium is formulated in combination with a pharmaceutically acceptable carrier, diluent, and/or excipient.

A second aspect of the invention features a composition that includes the mycobacterium of the first aspect of the invention in combination with a pharmaceutically acceptable carrier, diluent, and/or excipient. In an embodiment, the composition further includes an adjuvant.

A third aspect of the invention features a vaccine that includes the mycobacterium of the first aspect of the invention or the composition of the second aspect of the invention, in which the mycobacterium is capable of inducing an immune response in a mammal (e.g., a human or other mammal) against the epitope, antigen, and/or polypeptide or priming an immune response in a mammal against the epitope, antigen, and/or polypeptide. In yet another embodiment, the vaccine is for use in prophylaxis against or treatment of a pathogenic infection (e.g., human immunodeficiency virus (HIV) or Mycobacterium spp. (e.g., M. tuberculosis)), cancer, an allergy, an autoimmune disease, and/or graft rejection.

A fourth aspect of the invention features a method of inducing an immune response in a mammal (e.g., a human or other mammal) against an antigen by administering a composition that includes at least one mycobacterium of the first aspect of the invention, the composition of the second aspect of the invention, and/or the vaccine of the third aspect of the invention. In an embodiment, the composition includes a dosage of about 1×10³ to about 1×10¹² CFU of the mycobacterium. In yet other embodiments, the mammal is administered a single dose or a plurality of doses (e.g., 2, 3, 4, 5, 6, 7, 8, 9, 10, or more doses). In another embodiment, the plurality of doses are administered at least one day apart. In other embodiments, the composition is at least two weeks apart (e.g., at least 3, 4, 5, 6, 7, 8, 9, or 10 weeks apart). In still other embodiments, the method includes using the at least one mycobacterium of the first aspect of the invention, the composition of the second aspect of the invention, and/or the vaccine of the third aspect of the invention in a prime/boost protocol to induce an immune response in a mammal. In other embodiment, the at least one mycobacterium of the first aspect of the invention, the composition of the second aspect of the invention, and/or the vaccine of the third aspect of the invention can be used alone as an immunizing composition or as a priming vector, or the at least one mycobacterium of the first aspect of the invention, the composition of the second aspect of the invention, and/or the vaccine of the third aspect of the invention can be used in combination with a boosting composition, such as a recombinant adenovirus vector (rAd) expressing an antigen, epitope, and/or polypeptide for which an immune response is sought to be generated, a NYVAC vector expressing an antigen, epitope, and/or polypeptide against which an immune response is sought to be generated (see, e.g., Tartaglia et al., Virology 188(1):217-232, 1992), or a composition that includes an antigen, epitope and/or polypeptide (e.g., a protein antigen) against which an immune response is sought to be generated.

A fifth aspect of the invention features a kit for immunizing a mammal against a disease or disorder or for priming a mammal for an immune response against a disease or disorder. The kit includes at least one mycobacterium of the first aspect of the invention, the composition of the second aspect of the invention, and/or the vaccine of the third aspect of the invention, and instructions for their use. The at least one mycobacterium of the first aspect of the invention, the composition of the second aspect of the invention, and/or the vaccine of the third aspect of the invention can be used alone as an immunizing composition or as a priming vector, or the at least one mycobacterium of the first aspect of the invention, the composition of the second aspect of the invention, and/or the vaccine of the third aspect of the invention can be used in combination with a boosting composition, such as a recombinant adenovirus vector (rAd) expressing an antigen for which an immune response is sought to be generated, a NYVAC vector expressing an antigen against which an immune response is sought to be generated (see, e.g., Tartaglia et al., Virology 188(1):217-232, 1992), or a composition that includes an antigen (e.g., a protein antigen against which an immune response is sought to be generated).

A sixth aspect of the invention features a method of optimizing a mycobacterial vaccine vector by: a) mutagenizing a parental mycobacterial strain (e.g., Mycobacterium bovis BCG, such as M. bovis BCG Danish) expressing an antigen by disruption of one or more genes of the parental mycobacterial strain using a transposon to produce a mutated mycobacterium; and b) assaying the mutated mycobacterium for MHC class I presentation of the antigen, in which an increase in MHC class I presentation of the antigen relative to the parental mycobacterial strain indicates the mutated mycobacterium is an optimized mycobacterial vaccine vector. In an embodiment, the method further includes: c) assaying (e.g., using a tetramer assay) the mutant mycobacterium for a MHC class I-restricted T cell response (e.g., a CD8+ T cell response) against the antigen, in which an increase in the MHC class I-restricted T cell response against the antigen relative to the parental mycobacterial strain indicates the mutated mycobacterium is an optimized mycobacterial vaccine vector.

### BRIEF DESCRIPTION OF THE DRAWINGS

FIG. 1. Schematic representation of the in vitro screening of a transposon-mutagenized rBCG library for enhanced MHC class I presentation. Macrophages infected with rBCG-SIINFEKL transposon mutant strains (yellow) are compared to macrophages infected with the parental rBCG-SIINFEKL strain (blue) for their ability to stimulate IL-2 production by the RF33.70 T cell hybridoma. The presentation of SIINFEKL (SEQ ID NO: 1) on H-2K^{b} elicits IL-2 production by the RF33.70 hybridoma proportional to the number of SIINFEKL:H-2K^{b} complexes on the surface of the presenting A3.1A7 cells.

FIG. 2. IL-2 production by RF33.70 cells in response to exogenous SIINFEKL peptide over a range of concentrations.

FIG. 3. Schematic representation of the multicopy episomal pMV261-SIINFEKL plasmid illustrating the structure of the transgene.

FIG. 4. IL-2 production by RF33.70 in response to IFN-β-stimulated A3.1A7 cells infected with recombinant M. smegmatis expressing the 19 kDaSIINFEKL protein (rM. smeg-OVA) over a range of MOIs.

FIGS. 5A and 5B. Identification of novel rBCG transposon mutant strains that generate enhanced MHC class I-mediated presentation in vitro. FIGS. 5A and 5B are graphs showing representative results of IL-2 production by the T cell hybridoma RF33.70 stimulated by the presentation of SIINFEKL from A3.1A7 cells infected with various rBCG transposon mutant strains. The dotted line indicates the level of IL-2 production in response to the parental BCG-SIINFEKL strain.

FIG. 6. Primary CD8+ T cell responses induced in vivo by novel rBCG strains that generate enhanced MHC class I presentation in vitro. (A) Primary SIINFEKL-specific CD8⁺ T cell responses in C57Bl/6 mice inoculated IV with 1×10⁷ CFU of selected rBCG strains that generated enhanced MHC class I-mediated SIINFEKL presentation in vitro. (B) A second experiment testing different mutants.

FIG. 7. Secondary CD8+ T cell responses induced in vivo by boosting with rAd-SIINFEKL. Secondary responses in mice primed with selected rBCG transposon mutant strains. SIINFEKL-specific CD8⁺ T cell responses were assessed 7 days after boosting with a suboptimal dose of rAd-OVA (1×10⁶ vp) IM in mice primed with the indicated rBCG strains.

FIG. 8. Comparison of selected mutants C57 and J13 to AERAS 401 for their ability to generate SIINFEKL-specific CD8+ T cell responses. (A) Western blot for SIINFEKL expression from AERAS 401, a perfringolysin O-expressing strain of BCG engineered to express SIINFEKL from the pMV261-19 kDaSIINFEKL plasmid. (B)ᵣBCG mutants C57 and J13 were compared to recombinant AERAS 401 expressing SIINFEKL for their ability to generate SIINFEKL-specific CD8+ T cell responses.

FIG. 9. Persistence of novel rBCG strains in vivo. C57Bl/6 mice were inoculated with 1×10⁷ CFU of the indicated rBCG strains. Three and six weeks post-inoculation, mice were sacrificed and bacterial burden was assessed ex vivo. Bacterial burdens in spleen, liver and lung are indicated for each individual mouse.

FIG. 10. Complementing plasmid pYUB1141-K14 contains a region of the BCG genome including gene BCG_{—}1790. A region of genomic DNA spanning the site of the K14 transposon mutation was cloned into the integrating plasmid pYUB 1141.

FIG. 11. Complementing plasmid pYUB1141-AZ11 contains a region of the BCG genome including genes echA18 and amiD. A region of genomic DNA spanning the site of the AZ11 transposon mutation was cloned into the integrating plasmid pYUB 1141.

FIG. 12. Genetic complementation of novel rBCG strains K14 and AZ11. (A) Culture PCR reactions were performed to amplify a fragment of the BCG_{—}1790 gene from BCG wild type, BCG-SIINFEKL, K14, and the complemented strain of K14. (B) Culture PCR reactions were performed to amplify a fragment of the BCG_{—}3445 gene (echA18) from BCG wild type, BCG-SIINFEKL, AZ11, and the complemented strain of AZ11.

FIG. 13. Complementation of novel rBCG strains reduces CD8+ T cell immunogenicity. (A) SIINFEKL-specific T cell responses induced by the K14 rBCG strain are greater than those induced by the parental rBCG strain. Complementation of the K14 rBCG strain with the BCG_{—}1790 gene reduces the elicited SIINFEKL-specific CD8+ T cell responses. (B) SIINFEKL-specific CD8+ T cell responses induced by the AZ11 strain of rBCG are greater than those induced by the parental strain. Complementation of the AZ11 strain of rBCG with the echA18 and amiD genes reduces the elicited SIINFEKL-specific CD8+ T cell responses.

FIG. 14. Locations of mutations in the mycobacterial genome. FIG. 14 is a schematic showing the locations of mutations in the mycobacterial genome that give rise to increased transgene product-specific CD8+ T cell responses.

FIGS. 15A and 15B. Immunogenicity of selected rBCG mutant strains compared to plasmid DNA vaccine. Vaccination with rBCG mutants AF25 (ICO K) and J13 (ICO B) was compared with vaccination with plasmid DNA for elicitation of SIINFEKL-specific CD8+ T cell responses. As is shown in FIG. 15A, rBCG mutants AF25 (ICO K) and J13 (ICO B) generated greater SIINFEKL-specific CD8⁺ T cell responses than recombinant AERAS 401 expressing SIINFEKL. FIG. 15B is a graph showing that primary SIINFEKL-specific CD8⁺ T cell responses to rBCG mutants AF25 (ICO K) and J13 (ICO B) were comparable to the peak primary response elicited by vaccination with plasmid DNA. Peak tetramer responses occurred on day 7 following inoculation with the rBCG constructs and day 14 following inoculation with the plasmid DNA construct. Data are presented as mean±SEM, p values were determined by student's t test (* P<0.05, ** P<0.01).

FIG. 16. CD8+ T cell responses of rBCG- and plasmid DNA-primed mice following rAd5-boost immunization. Comparison of rBCG mutant C57-SIINFEKL with plasmid DNA-SIINFEKL for their ability to prime for a rAd5 boost. The various groups of primed mice were boosted with the suboptimal dose of 10⁶ PFU of rAd5-SIINFEKL and SIINFEKL-specific CD8+ T cell responses were assessed 11 days later by SIINFEKL-H-2K^{b} tetramer staining. C57 (ICO K)-primed, rAd-boosted SIINFEKL-specific CD8 T cell responses were comparable to DNA-primed, rAd-boosted responses and greater than recombinant AERAS 401-primed, rAd-boosted responses in mice. Secondary SIINFEKL-specific CD8⁺ T cell responses were tested on day 10 following boosting with a suboptimal dose of rAd5-SIINFEKL. Data are presented as mean±SEM, p values were determined by student's t test (* P<0.05, ** P<0.01).

FIG. 17. Construction of an AES to reproduce in wild type BCG Danish the gene deletions identified in the rBCG mutants AF25 and C57. (A) Transposons in the mutant rBCG strains AF25 and C57 were both mapped to the operon BCG_{—}2587-BCG_{—}2590. (B) An AES, pAES2589-Operon, was created to delete the entire BCG_{—}2587-BCG_{—}2590 operon. (C) Correct cloning of the AES was confirmed by digestion with EcoRI and sequencing of the homologous L and R arms (left panel). PacI digestion of the phasmid containing the allelic exchange substrate pAES2589-Operon confirmed the presence of the 47 kb phasmid phAE159 and the 6.5 kb AES (right panel).

FIG. 18. Construction of an AES to reproduce in wild type BCG Danish the gene deletion identified in the rBCG mutant J13. (A) The transposon in mutant rBCG strain J13 was mapped to the gene cmaA2 in the operon BCG_{—}0546c-BCG_{—}0547c. (B) An AES, pAES0546c-Gene, was created to delete the gene cmaA2 (BCG_{—}0546c). (C) Correct cloning of the AES was confirmed by digestion with Van91I and sequencing of the homologous L and R arms (left panel). PacI digestion of the phasmid containing the allelic exchange substrate pAES0546c-Gene confirms the presence of the 47 kb phasmid phAE159 and the 6.6 kb AES (right panel).

FIG. 19. Successful deletion of the BCG_{—}2587-BCG_{—}2590 operon in wild type BCG Danish. An AES packaged into the phasmid phAE2589-Operon was introduced into wild type BCG Danish using specialized transduction. (A) Deletion of the operon was demonstrated by PCR using primers that amplify a region of the BCG_{—}2588 gene and by primers that amplify a region of the BCG_{—}2589 gene. Primers that amplify PPE41, present in slow growing mycobacteria, confirmed successful PCR reactions. Primers that amplify the hygroR gene present in the AES demonstrated the successful introduction of the AES into the bacterium. Lane 1-wild type BCG, 2-BCG-SIINFEKL, 3-C57-SIINFEKL, 4-AF25-SIINFEKL 5-AF25Rec, 6-AF25Rec-SIINFEKL colony 1, 7-AF25Rec-SIINFEKL colony 2. (B) Southern blot data, probing with a probe specific to the gene BCG_{—}2589, demonstrates the presence of BCG_{—}2589 in wild type BCG Danish and the strain J13Rec, but the elimination of the BCG_{—}2589 gene in the strain AF25Rec. FIG. 19B confirms successful deletion of the operon BCG_{—}2587-2590 based on hybridization with a 306 bp probe specific for the gene BCG_{—}2589. The presence of BCG_{—}2589 was demonstrated in wild type BCG, but the gene was eliminated in the strain ICO K Rec. Equal loading of genomic DNA is demonstrated in the left panel by ethidium bromide staining of the gel.

FIG. 20. Successful deletion of the cmaA2 gene in wild type BCG Danish. An AES packaged into the phasmid phAE0546c-Gene was introduced into wild type BCG Danish using specialized transduction. (A) Deletion of the cmaA2 gene was demonstrated by PCR using primers that amplify a region of the cmaA2 gene. Primers that amplify PPE41, present in slow growing mycobacteria, confirmed successful PCR reactions. Primers that amplify the hygroR gene present in the AES demonstrated the successful introduction of the AES into the bacterium. Lane 1-BCG wild type, 2-BCG-SIINFEKL, 3-J13-SIINFEKL, 4-J13Rec-SIINFEKL colony 1, 5-J13Rec-SIINFEKL colony 2. (B) Southern blot data, probing with a 295 bp BCG_{—}0546c-specific (cmaA2-specific) probe, demonstrated that BCG_{—}0546c was not disrupted in another mutant (BCG transposon strain A25) or in wild type BCG Danish, but was eliminated in the strain J13Rec organism and was disrupted in the J13 transposon mutant rBCG strain.

FIG. 21. Novel rBCG strains AF25Rec and J13Rec expressing SIINFEKL elicited higher SIINFEKL-specific CD8+ T cell responses than wild type BCG Danish expressing SIINFEKL. (A) The plasmid pMV261-19 kDaSIINFEKL was cloned into BCG Danish, J13Rec and AF25Rec. Western blot analysis showed comparable expression of the 19 kDaSIINFEKL (20 kDa) protein by all the strains. (B) The reconstructed mutant rBCG strains AF25Rec and J13Rec elicited SIINFEKL-specific CD8+ T cell responses comparable in magnitude to their respective mutant strains selected from the transposon library.

FIG. 22. SIV Gag is secreted from rBCG constructs. (A) The SIV gag-containing plasmids pSL10 and pSL7 were cloned into the J13Rec and AF25Rec strains of BCG and cell lysates were analyzed by Western blot for SIV Gag protein. (B) Secretion of SIV Gag as assayed by p27 ELISA demonstrates comparable secretion among BCG constructs containing the pSL7 plasmid and comparable secretion among BCG constructs containing the pSL10 plasmid.

FIG. 23. Novel rBCG strains AF25Rec and J13Rec expressing SIV Gag elicited higher SIV Gag epitope-specific CD8+ T cell responses than wild type BCG Danish expressing SIV Gag. (A) The novel constructs AF25Rec and J13Rec expressing an Ag85a-SIV Gag fusion protein from pSL10 primed for higher Gag-specific CD8+ T cell responses upon suboptimal rAd5-SIV Gag boost than wild type BCG Danish expressing Ag85a-SIV Gag. (B) The novel construct J13Rec and the transposon mutant J13 expressing a 19 kDa-SIV Gag fusion protein from pSL7 primed for increased Gag-specific CD8+ T cell responses upon suboptimal rAd5-Gag boosting. These levels were comparable to the Gag-specific CD8+ T cell responses elicited by a plasmid DNA-SIV Gag/Ad5-SIV Gag vaccination regimen.

FIG. 24. Gene deletions associated with enhanced MHC class I-restricted transgene product-specific CD8+ T cell responses identified in a two-tiered screen of rBCG transposon mutants. FIG. 24 is a table showing disrupted genes in mutant rBCG strains that were associated with increased MHC class I presentation in vitro and increased transgene product-specific CD8+ T cell responses.

FIGS. 25A and 25B. Identification of novel rBCG transposon mutant strains that generate increased CD8⁺ T cell responses in vivo. FIG. 25 B is a graph showing primary SIINFEKL-specific CD8⁺ T cell responses generated in the peripheral blood following vaccination with 10⁷ CFU of selected novel rBCG mutant clones. Clones shown here are a subset of those that generated enhanced MHC class I-mediated SIINFEKL presentation in vitro relative to parental rBCG. FIG. 25B is a graph showing secondary SIINFEKL-specific CD8⁺ T cell responses in mice primed with selected rBCG transposon mutant clones. SIINFEKL-specific CD8⁺ T cell responses were assessed 10 days after boosting with a suboptimal dose of rAd-SIINFEKL (10⁶ vp) in mice primed with the indicated rBCG strains. Data are presented as mean±SEM.

FIG. 26. In vitro assessment of MHC class I presentation. Table summarizing in vitro and in vivo screening results for all 3290 rBCG strains tested.

FIGS. 27A and 27B. Novel rBCG strains ICO K Rec and ICO B Rec expressing SIINFEKL conferred greater protection against a heterologous Listeria challenge. FIG. 27A is a graph showing that ICO B Rec generated a SIINFEKL-specific CD8⁺ T cell response that was significantly greater than the response generated by wild type BCG expressing SIINFEKL. FIG. 27B is a graph showing that ICO B Rec provided significantly greater protection than BCG or BCG-SIINFEKL against a rLM-OVA challenge. Data are presented as mean±SEM, p values were determined by student's t test (** P<0.01).

### ACRONYMS AND ABBREVIATIONS

Ad—Adenovirus
AERAS—Global tuberculosis foundation
AES—Allelic Exchange Substrate
APC—Antigen Presenting Cell
BCG—Bacillus Calmette Guerin
CD—Cluster of Differentiation
CFU—Colony Forming Units
CMA—Cyclopropane Mycolic Acid
CTL—Cytotoxic T Lymphocyte
CR—Complement Receptor
DDM—Dideoxymycobactin
DI—Dairen I vaccinia virus
DNA—Deoxyribonucleic Acid
ELISA—Enzyme-linked Immunosorbent Assay
ELISPOT—Enzyme-Linked Immunosorbent Spot
ER—Endoplasmic Reticulum
Fc—Fragment, Crystallizable region of antibody
IFN—Interferon
kDa—KiloDalton
LAM—Lipoarabinomannan
mAb—Monoclonal Antibody
MHC—Major Histocompatibility Complex
MOI—Multiplicity of Infection
NETS—Neutrophil Extracellular Traps
OD—Optical Density
PAMPS—Pathogen Associated Molecular Patterns
PBMC—Peripheral Blood Mononuclear Cells
PCR—Polymerase Chain Reaction
PDIM—Phthiocerol Dimycocerosate
PE—Proline, Glutamic acid
PFU—Plaque Forming Units
PG—Peptidoglycan
PPD—Purified Protein Derivative
PPE—Proline, Proline, Glutamic acid
RD—Region of Difference
RPMI—Media developed at Roswell Park Memorial Institute
SIINFEKL—Immunodominant peptide from chicken ovalbumin
SCID—Severe Combined Immunodeficiency
SFC—Spot Forming Cells
SMAC—Supramolecular Activation Clusters
U—Units
UNICEF—United Nations Children's Fund

### DEFINITIONS

An “antigen” refers to any agent, generally a macromolecule, which can elicit an immunological response in an individual. As used herein, “antigen” is generally used to refer to a polypeptide molecule or portion thereof which contains one or more epitopes. Furthermore, for the purposes of the present invention, an “antigen” also includes a polypeptide having modifications, such as deletions, additions, and substitutions (generally conservative in nature) to the native sequence, so long as the polypeptide maintains sufficient immunogenicity. These modifications may be deliberate, for example through site-directed mutagenesis, or may be accidental, such as through mutations of hosts which produce the antigens.

An “immune response” against an antigen of interest is the development in a mammalian subject (e.g., a human) of a humoral and/or a cellular immune response to that antigen. For purposes of the present invention, a “humoral immune response” refers to an immune response mediated by antibody molecules, while a “cellular immune response” is one mediated by T-lymphocytes and/or other white blood cells. A mammalian subject to be treated with a BCG vector of the invention may be any member of the subphylum cordata, including, without limitation, humans and other primates, including non-human primates, such as chimpanzees and other apes and monkey species; farm animals such as cattle, sheep, pigs, goats and horses; domestic mammals, such as dogs and cats; laboratory animals including rodents, such as mice, rats and guinea pigs; birds, including domestic, wild and game birds, such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly. If a mammal, the subject will preferably be a human, but may also be a domestic livestock, laboratory subject or pet animal.

As used herein, the term “epitope” generally refers to the site on a target antigen which is recognised by an immune receptor such as a T-cell receptor and/or an antibody. Preferably it is a short peptide derived from or as part of a protein. However the term is also intended to include peptides with glycopeptides and carbohydrate epitopes. A single antigenic molecule may include several different epitopes.

### DETAILED DESCRIPTION OF THE INVENTION

### I. Introduction—Mycobacteria as Live Recombinant Vaccine Vectors

Vaccines have had a profound impact on the control of infectious disease. The development of live recombinant vectors in the past three decades has greatly advanced the field of vaccinology and immunology. Novel live recombinant vectors will most likely be a source of future vaccines for emerging infectious agents and complex pathogens for which traditional vaccines have provided inadequate protection.

Since the initial creation of a live recombinant pox virus delivering influenza hemagglutinin, recombinant vaccine vectors have been created out of a diversity of viruses and bacteria [1]. Key features of recombinant vectors are in vivo replication, induction of a memory immune response, a genome capable of tolerating large foreign genes, and expression of those foreign genes in a form that is nearly identical to that expressed under natural conditions [2]. In addition, a successful recombinant vector must be cost effective, have a good safety profile, have high and durable expression of the transgenic protein, and have the ability to induce a robust transgene product-specific immune response at the site of infection of the pathogen [3].

Each specific live vector stimulates the immune response in a unique fashion based upon its life cycle in the host, the pathogen associated molecular patterns (PAMPS) it contains, and the limited pathology it causes. Some vectors induce a stronger humoral immune response, some vectors induce a stronger cellular immune response, and some vectors induce both types of responses. Each pathogen is controlled by the immune response in a particular way, so it is necessary to select a vector for creating a vaccine that induces an immune response appropriate for the control and clearance of the specific pathogen [4]. Also, as there is often a correlation between pathogenicity of a vector and its immunogenicity, it is important to balance potent immunogenicity and a tolerable safety profile.

One live vector, recombinant Mycobacterium bovis BCG (rBCG), stands out as a potential vaccine vector for the stimulation of cellular immune responses. The original application of BCG was to combat tuberculosis. However, through the expression of foreign transgenic proteins, there are a large number of infectious agents in humans for which one could use rBCG. rBCG is a strong T cell-inducting vaccine vector because of its unique biological properties; specifically, it contains many highly immunogenic lipoproteins and is an intracellular pathogen capable of surviving in macrophages and continuously synthesizing proteins. Therefore, rBCG is an attractive delivery vector because it is highly immunogenic and can persist within the host where it replicates and produces a transgene product. The safety profile of BCG has been borne out by the fact that it has been given to approximately 3 billion individuals with minimal adverse effects [2]. In addition, rBCG has the potential to stimulate a large cellular immune response skewed to a Th1 phenotype that is targeted to the mucosal area [5, 6]. Furthermore, when combined with a protein boost, rBCG vaccines have demonstrated the ability to enhance transgene protein-specific antibody responses [7, 8].

The ability to generate robust cytotoxic T lymphocyte (CTL) and T helper cell responses in combination with antibody responses, all targeted to mucosal surfaces, suggest that rBCG has potential as an effective vaccine vector. However, as a derivative of the pathogenic M. bovis strain, rBCG contains genes that limit the effective immune response against a transgenic protein. First generation recombinant BCG vaccines have been created and tested in simian models and human clinical trials with some success [9-12]. The robust BCG vector-specific responses suggest the potential of this vaccine, although responses of the same magnitude against a transgenic protein have not been realized. There is good reason to believe that the limitations that exist in using rBCG as a vector vaccine reflect the biology of BCG, and that elimination of these limitations would substantially increase the potential of BCG as a vaccine vector. There is need to engineer BCG so as to address these limitations and harness BCG as a vaccine vector. Understanding the biology of BCG and the nature of the immune response generated by this organism is a crucial first step to understanding how to improve its immunogenicity.

### 1.1 BCG History

One hundred years ago there was pressing need for a tuberculosis vaccine. Tuberculosis, known as consumption and the white plague in the early 1900's, was responsible for over 40% of deaths in European cities. In 1880, Robert Koch identified the etiological agent for tuberculosis as a bacillus he named Mycobacterium tuberculosis. While sanitary measures had a profound effect on tuberculosis control, they were insufficient to eliminate the disease. Koch had designed a subunit vaccine that he claimed could cure tuberculosis; however, clinical trials data indicated it was a failure [13].

At the time, successful vaccines were being developed by using microbes that were close relatives of human pathogens; these organisms contained many homologous antigens but were less pathogenic than the human pathogens. Work by Edward Jenner had demonstrated successful prophylactic vaccination against smallpox using the similar but less pathogenic strain of cow pox. Attempts to vaccinate against M. tuberculosis using M. bovis isolated from the milk of a cow suffering from tuberculous mastitis proved unsuccessful, as M. bovis is also highly pathogenic in humans. Both M. tuberculosis and M. bovis are members of the paratuberculosis complex, a collection of seven species that include M. canettii, M. africanum, M. pinnipedii, M. microti, and M. caprae. These species of mycobacteria all cause similar pathologies in mammalian hosts [14]. The genomic sequence of M. bovis differs from M. tuberculosis by as little as 0.05%, and therefore is quite similar [15-17].

Albert Calmette and Camille Guerin made the observation that M. bovis changed morphology and was slightly attenuated over the course of 15 serial in vitro passages on ox bile and glycerol-soaked potato slices. They devised a radical approach that would result in a novel vaccine: to genetically modify M. bovis by serial in vitro passage, making it less pathogenic yet still able to confer protection against tuberculosis. For 13 years, Calmette and Guerin grew M. bovis on potato slices soaked in glycerol and ox bile. During this time, they periodically checked for strains that were less pathogenic. By the end of 13 years, they were satisfied that they had selected for an attenuated M. bovis strain that had lost in vivo pathogenicity while growing in vitro.

Until the development of modern molecular techniques and full genomic sequencing, the new phenotype of this strain was evident while the genetic changes responsible for this phenotype were not. Now, through the full genome sequencing of this strain of M. bovis, we know that approximately 133 genes were lost during this 13 year culture period, although the main mechanism of attenuation has been attributed to the loss of one operon [18, 19]. This region, termed the Region of Difference 1 (RD1), encodes both effector proteins and a novel secretion pathway to pump these effectors out of the cell. The effector proteins, identified as Cfp-10 and Esat-6, form a complex that is capable of puncturing a hole in the vesicle holding the bacillus, allowing it to escape into the host cell's cytosol. Loss of this region from M. bovis and M. tuberculosis is associated with a dramatic loss of pathogenicity [20, 21].

The new strain of M. bovis, M. bovis Bacillus Calmette Guerin, was distributed throughout the world as a vaccine for tuberculosis after 230 in vitro passages. Between 1921 and 1961, another 943 passages occurred, further attenuating the strain. Until the advent of cryopreservation in 1961 and the establishment of a seed lot system by the World Health Organization in 1966, strains of BCG at different institutions were kept continuously growing, causing them to diverge genetically from each other [22].

During the second period of attenuation from 1921-1966, deletions, duplications, and point mutations arose that led to a genetic divergence in the strains kept at different institutions. Of particular significance, a second Region of Difference (RD2) deletion occurred in strains that were acquired after 1927, including BCG Danish and BCG Pasteur. The RD2 deletion leads to decreased M. tuberculosis proliferation within the host macrophage and decreased ability to modulate the host immune response [23]. At about the same time, a substitution mutation arose in BCG strains Pasteur, Danish, and others that were obtained after 1927. This substitution results in a 98_{glycine}_{_{→}}_{aspartic acid} substitution that inactivates the mmaA3 gene and leads to a loss of methoxymycolate production [24]. This loss of methoxymycolate expression appears to have no impact on BCG pathogenicity [25, 26].

The large genetic differences between BCG strains has been shown to lead to a large variation in gene expression [18], a large range in immunogenicity, and a large range in the efficacy of each strain as a vaccine for tuberculosis. This difference has been acknowledged by the vaccine community, although there is no consensus as to the optimal strain for use as a tuberculosis vaccine. Currently, BCG Danish is one of the three most commonly administered strains by UNICEF [27].

It has been estimated that over 3 billion doses of BCG have been administered, with over 100 million given yearly [2]. The efficacy of BCG as an adult pulmonary tuberculosis vaccine has been quite varied, ranging from 1-80%. This variation has been attributed to human exposure to non-pathogenic environmental mycobacteria, the strain of BCG used during vaccination, the freeze drying preparation of the BCG, and variations in the infecting strains of M. tuberculosis. However, while BCG shows limited and variable protection against adult pulmonary tuberculosis, it has proven more consistent and efficacious against severe forms of tuberculosis; BCG is over 80% efficacious against childhood miliary tuberculosis and tuberculosis meningitis [2].

### 1.2 Immune Responses Generated to BCG

The utility of BCG as a vaccine vector is a consequence of its highly immunogenic nature. In analyzing the immune responses generated to a live rBCG vector, it is important to examine both the immune response that clears the bacteria as well as the range of immune responses that are generated during infection. A broad range of immune responses are generated during mycobacterial infection, but not all contribute significantly to clearance of the bacteria. However, while these responses may not be responsible for clearing the bacteria, they still may be functional. In the creation of a vaccine, it is possible to harness the full range of immune responses generated by a vector [28].

1.2.1 Innate Activation

A typical vaccine inoculum of BCG contains 10⁵-10⁶ viable organisms, and is administered intradermally at the insertion of the deltoid in the upper arm of infants days to weeks after birth [29-31].

The innate immune response that occurs at the site of inoculation has a profound impact on the ensuing adaptive immune response, yet these events are poorly characterized. There is an immediate influx of first-responding neutrophils at the site of inoculation [32]. Neutrophils are capable of phagocytosing bacilli, becoming activated, and then killing these bacteria [33]; however, they have also been shown to apoptose and limit mycobacterial spread through the formation of neutrophil extracellular traps (NETS) [34]. Neutrophils are the predominant cell type responsible for early transport of bacilli and antigen to the draining lymph nodes [35]. Neutrophils containing live mycobacteria apoptose, and professional antigen presenting cells (APC), such as macrophages and dendritic cells, take up the apoptotic particles containing bacteria.

Macrophages and dendritic cells are also rapid responders to the site of BCG inoculation and are critical to the stimulation of robust adaptive cellular immune responses. For this reason, pathogenic mycobacteria have evolved numerous ways of inhibiting normal processing of internalized bacteria. Resident dendritic cells, dendritic cells derived from peripheral blood monocytes [36], and macrophages are all capable of internalizing bacilli.

Internalization by macrophages has been shown to be a receptor mediated, actin-dependent process. Specifically, pathogenic mycobacteria coated by complement fragments C3b and C3bi are taken up by complement receptors CR1, CR3, and CR4 [37]. Work using human macrophages has demonstrated that pathogenic mycobacteria can be taken up directly through the interaction of lipoarabinomannan (LAM) with the Mannose Receptor (CD206) [38, 39]. In the murine system, a different scavenging receptor (CD36) is necessary for optimal uptake of BCG [40]. Multiple other receptors are capable of binding to mycobacteria, including CD14 binding to LAM and Fcγ receptors binding to mycobacteria coated with IgG antibody. These examples illustrate the many and complex mechanisms responsible for mycobacteria entry into host phagocytes [41].

Significant activation of these phagocytic cells occurs through Toll Like Receptor (TLR)-ligand interactions. Mycobacterial lipoproteins and peptidoglycan bind to TLR-2, and unmethylated CpG DNA activates TLR-9 [6, 42]. Complement also has a role in activating macrophages and neutrophils to undergo a respirative burst; the complement anaphylatoxin C5a binds to C5a receptor (CD88), which is upregulated during BCG infection, stimulating an oxidative burst capable of bactericidal activity [43].

Following pattern recognition receptor ligation during the course of an infection, the APC shuttles peptide-loaded MHC class I and MHC class II molecules to the surface to provide “signal 1” to T cells. It also upregulates expression of key coreceptors including B7.1 and B7.2 to provide “signal 2” to T cells.

1.2.2 Phagosome Subversion

Normal macrophage activity involves the progressive acidification of the phagosomal vesicle containing the bacteria, fusion of this vesicle with the lysosomal compartment, and the introduction of hydrolytic molecules and enzymes including inducible nitric oxide synthase (iNOS) and phagocyte oxidase NOX2/gp91phox [44]. Once internalized in the phagosome, M. tuberculosis and M. bovis BCG alter the phagosomal membrane to prevent phagosomal-lysosomal fusion, prevent acidification of the vesicle, and replicate in this new permissive environment [45]. Through direct contact with the phagosomal membrane, these bacteria are capable of influencing the host proteins and enzymes in the phagosomal membrane [46]. Phthiocerol dimycocerosates (PDIM) on the surface of pathogenic mycobacteria have been shown to be inserted into the phagosomal membrane and limit the acidification of the vesicle [47]. Differential localization of Rab GTPases 5, 7, and 10, which direct vesicle transport and fusion, have been found on the surfaces of mycobacterium-containing vesicles [48, 49]. Of note, Rab7, which directs phagosomes for fusion with late endosomes, is found in an inactive, GDP-bound state on the surface of BCG-containing phagosomes [50]. The inappropriate localization and inactivation of the Rab family of GTPases prevents the fusion of lysosomes with the BCG-bearing phagosomes, allowing the mycobacteria to survive within the host cell.

In addition, M. tuberculosis has also been shown to destabilize the phagosomal membrane in an RDI-dependent mechanism and escape into the host cytosol where it can undergo further replication. BCG, lacking the RD1 operon, cannot escape from the phagosome [51].

1.2.3 Antigen Presentation

Adaptive immune responses play a critical role in overcoming the processing and presentation block that is crucial to surviving a mycobacterial infection. CD4+ T helper cells provide critical cytokines for macrophage activation that can overcome the phagosome/lysosome fusion block and lead to killing of the bacteria. Indeed, the successful induction of a CD4+ T cell response is the single most important adaptive response for control and clearance of pathogenic mycobacteria, as mice deficient in MHC class II and CD4+ T cells die rapidly after infection [52]. CD8+ cytotoxic T cells are also necessary for protection against pathogenic mycobacterial infections, although mice deficient in β-2M and CD8+ T cells die after infection several weeks after their MHC class II-deficient counterparts [53]. CD8+ T cells are capable of directly lysing infected APCs using perforin, Fas-Fas ligand interactions, and TNF-R [54] as well as producing activating cytokines IFN-γ and TNF-α. IFN-γ is capable of stimulating alternative IFN-γ-inducible Rab GTPases on vesicles, which overcome the block that mycobacteria have established in infected cells, allowing for the fusion of the late endosome with lysosomes. IFN-γ also acts by activating inducible Nitric Oxide Synthase (iNOS) to produce nitrite and nitrate, which turn to nitric oxide in the acidic phagosomal compartment and kill internalized bacteria. [55, 56]. TNF-α enhances neutrophil killing of pathogenic mycobacteria [57]. In murine studies, protection conferred by BCG was correlated with rapid and large numbers of CD8+ T cells in M. tuberculosis-infected tissue [58]. In studies involving nonhuman primates, protection mediated by BCG vaccination against M. tuberculosis can be abrogated by CD8+ T cell depletion [59]. CD4+ T cell help at the time of primary mycobacterial infection is necessary for the generation of a cytotoxic CD8+ T cell memory population in response to mycobacteria [60, 61]. In the absence of CD4+ T cells, pathogenic mycobacteria are capable of undergoing exponential growth that is normally restricted to a stationary phase by the CD4+ T cell population [62]. For these reasons, antigen presentation to CD4+ and CD8+ T cells is crucial to a successful immune response to mycobacteria.

1.2.4 Mechanism of MHC Class II Presentation

Traditional foreign antigen taken up by APC through phagocytosis is degraded into peptides in the late lysosomal compartment. These compartments contain high levels of MHC class II in an immature form, with the peptide binding groove occupied by the chaperoning peptide invariant chain. Foreign antigen is processed and loaded onto MHC class II molecules in these late endosomal compartments, giving them the name MHC class II loading compartment (MIIC). Activation of the APC through pattern recognition receptors or activating cytokines causes the APC to cease phagocytosis and instead shuttle MHC class II: peptide complexes to the cell surface. Tubulation of the MIIC directs large numbers of these complexes to the surface for interaction with the TCR of CD4+ T cells [63]. In addition, the activation of the APC stimulates the cell to upregulate costimulatory molecules including CD40, B7.1, B7.2, molecules necessary for supramolecular activation cluster (SMAC) formation, and cytokines.

1.2.5 Mechanism of MHC Class I Presentation

Self-antigen and intracellular antigen found in the cytosol are presented on MHC class I molecules. These antigens are degraded in the proteosome and immunoproteosome and then shuttled from the cytosol into the endoplasmic reticulum lumen by TAP. At this point, the fragments are trimmed to 8-10 amino acid peptide fragments and loaded in the groove of the MHC class I molecule. From there, the MHC class I:peptide complexes are transported through the golgi apparatus to the cell surface.

During the course of active mycobacterial infection, secreted proteins from live bacilli gain access to the MHC class I and MHC class II presentation pathways [64, 65]. The paradigm of MHC class II loading predicts that proteins from mycobacteria in the phagosome will be loaded onto MHC class II molecules in the late endosomes/lysomes MIIC vesicle, however it is unclear how mycobacterial proteins gain access to the host's MHC class I pathway. Given that BCG within an intact phagosome are topologically separate from the traditional cytoplasmic areas that feed the MHC class I presentation machinery, it is unclear how these antigens move to the endoplasmic reticulum (ER) lumen where MHC class I presentation machinery exists. Data demonstrating phagosomal escape by RD1-containing M. tuberculosis, M. marinum, and M. leprae suggest a mechanism for the exit of antigen from the phagosome into the cytosol. Stronger CD8+ T cell responses have been measured to mycobacteria escaping from the phagosome, such as M. tuberculosis, compared to those mycobacteria that do not, such as BCG. Deletion of the RD1 region in M. tuberculosis has been shown to lower the CD8+ T cell response [66]. However, mycobacterial strains lacking the RD1 region are still able to generate MHC class I-restricted responses indicating that there are other mechanisms for mycobacterial proteins to access the MHC class I response. The generation of a CD8+ T cell response against BCG indicates that the lack of phagosomal escape in combination with the phagosomal/lysosomal block is insufficient to inhibit generating CD8+ T cell responses [67, 68].

1.2.6 Mechanisms of Cross Presentation

Cross presentation of antigen from the MHC class II pathway into the MHC class I pathway is one mechanism that explains the generation of a CD8+ T cell response. Cross presentation of exogenous antigen by bone marrow-derived APC and the subsequent generation of a CD8+ T cell response was demonstrated in 1990 [69]. Different mechanisms of cross presentation have been proposed, including retrograde transportation of peptide out from the endosome into the cytosol of the infected macrophage, processing of the MHC class I epitope and loading on MHC class I in the phagosome, and apoptosis of the infected macrophage and transfer of the antigens to uninfected dendritic cells. Several reports have indicated that murine macrophages derived from monocytes are unable to stimulate a CD8+ CTL response alone. Furthermore, loading of antigen onto MHC class I in ER-like endosomes was not observed in monocyte-derived macrophages [70]. These studies would suggest that mycobacteria residing in the early endosomes of macrophages are blocking endosomal/lysosomal fusion in part to evade detection. Unlike macrophages, dendritic cells are capable of loading exogenous particulate matter into MHC class I molecules within ER-like endocytic vesicles. Dendritic cells can also pump soluble antigen from the endosome to the cytosol and into the ER lumen for processing and loading onto MHC class I in the ER, albeit with a much lower level of efficiency of presentation compared to traditional MHC class I loading [70, 71].

A large body of literature would suggest that transfer of antigen from infected macrophage to dendritic cells primes the CD8+ T cell response. Two hypotheses exist to explain this transfer: exosomes shed from the infected macrophage are taken up by dendritic cells, and the infected macrophage undergoes apoptosis and apoptotic blebs containing mycobacterial antigens are taken up by dendritic cells. These dendritic cells then cross present antigen by the above mentioned mechanisms.

1.2.6.1 Cross Presentation Via Exosomes

Exosomes from infected macrophages are capable of stimulating CD4+ and CD8+ T cell activation. Exosomes are a product of exocytosis, secreted into the surrounding environment when early or late endosomal vesicles fuse with the cellular plasma membrane [72]. When exosomes are derived from endosomal compartments containing mycobacteria, mycobacterial antigens, especially glycolipoproteins, are incorporated in the exosome [73]. In addition, when derived from APC, exosomes contain both MHC class I and MHC class II molecules on their surface. These exosomes can stimulate DC activation and maturation as well as CD4+ and CD8+ T cell activation [74]. Exosomes generated from macrophages infected with BCG were capable of directly stimulating CD4+ and CD8+ T cells, but responses were much more potent when the macrophage-derived exosomes were first taken up by dendritic cells and then the antigen was presented to T cells.

1.2.6.2 Cross Presentation Via Apoptosis

In a mechanism that relies upon infected-host cell death, apoptotic vesicles carrying fragments of mycobacteria have been shown to stimulate an alternative “detour” pathway of cross presentation to CD8+ T cells [75]. In studies investigating this detour pathway, inhibition of apoptosis decreased CD8+ T cell priming. Pathogenic strains of M. tuberculosis are capable of sending APC down a necrotic death pathway by inhibiting macrophage synthesis of Prostaglandin E2 (PGE2) [76]. Without PGE2, the macrophages cannot repair damage to the plasma membrane and mitochondria, which shuttles the cell down a necrotic pathway avoiding apoptosis and avoiding subsequent T cell activation [77]. By avoiding apoptosis and instead diverting the host down a necrotic death pathway, the pathogen escapes significant T cell immune activation [78]. Attenuated and avirulent strains of mycobacteria, including BCG, induce an apoptotic death of infected macrophages that increases detour cross presentation and deny the bacteria a safe, protected environment in which they can reproduce [79, 80].

Mechanisms of cross presentation lead to the generation of very strong CD8+ T cell responses against several epitopes during mycobacterial infection. Responses against key secreted epitopes contained in Esat-6 and TB10.4 can be upwards of 40% of the total CD8+ T cell population. Despite the magnitude of these responses, they do not confer protection against M. tuberculosis. A vaccine used to generate robust CD8+ T cell responses specific for these same antigens prior to challenge with pathogenic M. tuberculosis did not have a protective effect [81]. Therefore, it is possible that these epitopes are being used as decoys by the mycobacteria in an effort to drive the immune response in a non-protective direction.

1.2.7 CD1 Presentation of Antigens

A mechanism of lipid presentation has been identified that may play a significant role in the immune response to mycobacteria. The CD1 family of molecules is capable of presenting lipoproteins and lipids by holding the long fatty-acid chains in deep grooves, while the head groups are exposed for T cell scanning. Four types of CD1 molecules (a, b, c, d) are capable of presenting lipids in humans, while in mice there is only one CD1 homologue expressed, CD1d. With the wide range of lipoproteins expressed by mycobacteria, it has been suggested that these molecules may have a critical role in stimulating an anti-mycobacterial immune response. Unlike the polygenic MHC molecules, CD 1 molecules are monogenic. Within humans, group 1 CD1 molecules (CD1a, CD1b, CD1c) have been identified that are capable of presenting mycobacterial lipoproteins such as dideoxymycobactin (DDM) to CD8+ T cells [82-84]. Together, these data would indicate that the CD1 locus may have evolved to combat mycobacterial infections. In fact, some studies have suggested that methods of mycobacterial evasion that are effective against traditional MHC processing are ineffective against CD1-restricted T cell responses, indicating that the role of CD1 molecules is not redundant but rather essential for mediating an immune response when traditional mechanisms are insufficient [85]. CD1d-restricted NKT cells show very rapid primary response kinetics, but poor recall responses [86]. Investigations studying recall responses to mycobacterial antigens complexed to the group 1 CD1 molecules that are present in humans have been limited by the lack of an appropriate model, as these CD1 molecules are not expressed in mice. Recent studies using mice that are transgenic for the group 1 CD1 molecules suggests that T cells restricted to these CDI respond with the same kinetics as traditional MHC class I-restricted CD8+ T cells [87]. As CD1-restricted T cells can recognize mycobacterial antigens, a rBCG vaccine may be able to stimulate this branch of the immune response.

1.2.8 Antibody Responses Generated by Mycobacteria

Antibody responses are also generated against several secreted proteins from pathogenic mycobacteria. While antibodies do not play an essential role in protection during the natural course of mycobacterial infection, they can contribute at some level to M. tuberculosis control [88]. B cell-deficient mice demonstrate increased susceptibility to M. tuberculosis, with several fold higher levels of colony forming units (CFU) in the lungs but not in the spleen or liver [89-91]. A protective effect of sera from M. tuberculosis-exposed mice was observed in preventing the reactivation of latent M. tuberculosis in a severe combined immunodeficiency (SCID) mouse model where primary infection was treated with chemotherapy. Serum from natural infection contains antibodies that can contribute to control of mycobacteria but by itself is not sufficient to control or clear mycobacteria. Antibody therapy using monoclonal antibodies has been successful at reducing systemic infection with M. tuberculosis, indicating that despite its intracellular status, M. tuberculosis is, at some point, exposed to antibodies [92]. Monoclonal antibodies (mAb) against the M. tuberculosis 16 kDa α-crystallin antigen, but not the 38 kDa antigen, have reduced bacterial load and led to less severe pathology compared to control immunized mice [93]. Similarly, passive immunotherapy with mAb against LAM reduces bacterial count in the lungs and spleen and increases survival time [94]. Most recently, a monoclonal IgA antibody against the 16 kDa α-crystallin antigen that was capable of binding to the FcaR1 IgA receptor reduced M. tuberculosis burden by 10-fold following bacterial challenge [95]. The control of M. tuberculosis by administered monoclonal antibodies is not indicative of the antibody response that a mycobacterial vector can generate. Antibody responses can be detected in approximately 50% of humans with active tuberculosis infection [96]. Rabbits immunized with BCG generated antibody responses to protein and polysaccharide antigens [97]. Specific investigation into these antigens indicates that antibodies are formed against a wide range of secreted and exposed proteins and carbohydrate antigens, including LAM [98, 99], antigen 60 (A60) [100], Ag85, the 16 kDa antigen [101], acylated trehalose antigen [102], mtb81 [103], and the 38 kDa antigen [104-107], yet the protection that all of these responses confer is unclear [108, 109].

Both CD8+ T cell and antibody responses are induced against secreted mycobacterial proteins, yet these responses are not capable of completely controlling pathogenic mycobacterial infections and the mycobacteria may be using these secreted proteins as decoys. For the purposes of using mycobacteria as vaccine vectors, it may be possible to harness the diversity of immune responses stimulated and generate strong CD8+ T cell responses and antibody responses directed against secreted transgenic proteins.

### 1.3 Immune System Evasion and Immunomodulation by Mycobacteria

Genetic analysis indicates that M. tuberculosis is 3 million years old and may have originated from environmental mycobacteria that acquired genes necessary to invade host cells [110, 111]. Tubercle scars on mummies (3000 B.C.) as well as mycobacterial lipid biomarkers on skeletal remains (7000 B.C) confirm that our earliest ancestors were infected with pathogenic strains of mycobacteria [112, 113].

It comes as no surprise then that pathogenic mycobacteria have developed a substantial number of genes dedicated to suppressing the mammalian host immune response. During the millions of years of tuberculosis and human evolution, there has been an ongoing host-pathogen arms race in which the host develops a method of eliminating the pathogen and the pathogen subsequently develops a way to avoid elimination [114]. In examining the literature on the effect that BCG has on suppressing or subverting the immune system, we have included data generated using M. tuberculosis for the cases in which M. bovis BCG has homologous genes.

1.3.1 Mycobacterial Inhibition of CD4+ T Cell Responses

In addition to blocking phagosomal/lysosomal fusion and preventing apoptosis, pathogenic mycobacteria have evolved a number of mechanisms to limit CD4+ T cell responses through the disruption of antigen presentation by MHC class II molecules. Functionally, monocytes and macrophages cultured with pathogenic mycobacteria lose their ability to stimulate CD4+ T cell activation. Gercken et al. describe work in which pathogenic mycobacteria act directly upon the cell they are infecting to reduce both the total amount of MHC class II on the cell surface as well as to limit T cell activation to an exogenous antigen [115]. The bacterium evades maximal CD4+ T cell responses by disrupting antigen processing, MHC class II expression, MHC class II trafficking, and MHC class II:peptide binding. Some reports conflict on the effects of mycobacteria on the MHC class II system. Because of the key role that timing has in generating a robust immune response, it is quite possible for investigators to examine the same phenomenon at different times, make different observations, and come to different conclusions. It is also possible that investigators generated conflicting data because they used different cell types, as cell lines may yield different results from monocyte-derived macrophages or other sources of APC. Cells obtained from different host organisms may also respond differently to pathogenic mycobacteria. Finally, conflicting observations may be made as a consequence of the use of different strains of mycobacteria to generate these data.

1.3.1.1 Mycobacterial Disruption of MHC Class II Expression

Immature, clip loaded, MHC class II molecules that are found in the MIIC compartment are newly synthesized. The pathogenic mycobacterial 19 kDa protein (LpqH) has been shown to bind to TLR-2 and trigger suppression of the Class II Transactivator (CIITA) protein by changes in histone acetylation [116]. The CIITA is considered the master regulator of MHC class II production, in part because of its direct effect upon the MHC class II enhanceosome. This complex modifies histones and remodels the promoter region of the MHC class II locus to regulate MHC class II mRNA [117-119]. Lower levels of CITTA generated by mycobacterial TLR-2 activation lead to lower levels of MHC class II mRNA [120, 121], and subsequently lower levels of MHC class II in the phagosome. LprA and LprG have been shown to have similar effects [122, 123].

One study, however, generated conflicting results. Hmama et al. observed unchanged levels of MHC class II mRNA in M. tuberculosis-infected cells [124]. They observed lower levels of MHC class II within the endosomal compartment. Because they found no difference in mRNA MHC class II transcripts, but lower levels of protein, they attributed the difference to atypical trafficking of the MHC class II molecules following synthesis.

1.3.1.2 Mycobacterial Disruption of MHC Class II Trafficking

Through the inappropriate kinetics of the host immune response, mycobacteria are able to generate a period of respite from the immune response long enough to gain a foothold in the host and begin reproducing. It has been demonstrated that mycobacteria prematurely activate MHC II-containing vesicles and consequently miss-time the protein loading of these complexes. Premature stimulation of dendritic cells during infection causes a cessation of antigen uptake and a shuttling of MHC class II from the MIIC to the surface. By prematurely activating dendritic cells to shuttle their MHC class II to the surface, only host proteins are available for presentation [85].

CD1 molecules are not subject to the same premature shuttling to the surface that disrupts traditional MHC class II presentation of peptides in the setting of mycobacterial infection. However, when dendritic cells are derived from monocytes in the presence of mycobacteria, these dendritic cells lack CD1 expression, make no IL-12, and activate T cells that are unable to express IFN-γ. This effect was attributed to the mycobacterial α-glucagon associated with the cell wall [125, 126].

1.3.1.3 Antigen Processing and Binding to MHC Class II

Noss et al. have reported that mycobacterial infection of macrophages decreases exogenous antigen catabolism within the phagosome. This catabolism of antigen is the first step of antigen processing, breaking the antigen into fragments of suitable size to be presented by MHC class II molecules [121]. By preventing the IFN-γ-induced activation of the cathepsin S molecule, mycobacteria are able to prevent the removal of the invariant chain [124, 127]. Furthermore, by decreasing levels of H2-DM within the phagosome, mycobacteria decrease removal of the clip protein and loading of MHC class II complexes with foreign peptide [121]. These mechanisms limit the processing of antigen and the loading of these peptide fragments into the MHC class II groove.

1.3.2 Mycobacterial Cytokine Modulation

Overexpression of key cytokines can create a less-than-optimal immune response. While TNF-α has crucial macrophage-activating properties, overexpression of TNF-α by macrophages at the time of mycobacterial infection allows more rapid growth of pathogenic mycobacteria, and represents another way that the bacteria may skew the immune response in an effort to gain a growth advantage [128].

1.3.3 Mycobacterial Inhibition of CD8+ T Cell Responses

Less data are available on the effect that pathogenic mycobacteria have on the presentation of MHC class I epitopes. Unlike in the MHC class II system, mycobacteria have no effect on expression of MHC class I molecules [129]. They also have no effect on the traditional mechanism of presentation of antigens located in the cytosol [129]. However, they do have an effect on the processing and loading of MHC class I molecules through the alternative MHC class I loading pathway, in which antigen in the phagosome is presented on MHC class I molecules [129]. In the absence of inhibitory molecules from mycobacteria, TLR-9 can be activated by pathogen-associated unmethylated CpG DNA, leading to the expression of IFN-α and β, which effectively enhance cross presentation. However, when TLR-2 activation occurs by the pathogenic mycobacterial 19 kDa lipoprotein, it leads to suppression of TLR-9-mediated expression of IFN-α and β, effectively limiting cross presentation [130]. It also leads to less phagosomal maturation, less delivery of lysosomal proteases, and less antigen processing in this compartment [129]. Through this mechanism, mycobacteria can limit MHC class I responses.

### 1.4 Existing BCG Strains

Many strains of modified BCG have been generated in the search for a more effective vaccine to prevent adult pulmonary tuberculosis. Rational approaches to the creation of an effective BCG vector are based upon the assumption that adding or deleting a key immunogenic protein should increase rBCG efficacy. However, implicit in almost all of these modified BCG vaccines for tuberculosis is that the high degree of similarity between the vector and the pathogen is key to generating protection. Potential tuberculosis vaccines have been made by modifying BCG in several ways, including the restoration of the M. bovis/M. tuberculosis genes deleted through attenuating passage, expression of highly immunogenic M. tuberculosis antigens, and expression of recombinant human cytokines by the BCG critical for productive and protective anti-tuberculosis responses.

Highly immunogenic M. tuberculosis or M. bovis proteins have been overexpressed, such as the 30 kDa mycolic acid transferase Ag85b or the gene Rv1767, which is upregulated during M. tuberculosis infection. Other modified rBCG vaccines carry foreign transgenes that perform a function not originally ascribed to BCG. These include rBCG strains that carry proteins to puncture the phagosome and give access to the cytosol, such as Esat-6, perfringolysis, and listeriolysin. Some investigators have attempted to bypass the BCG-host interface and directly skew the immune response by enabling rBCG to express a mammalian cytokine such as IL-2, IL-12, GM-CSF, TNF-α or the MHC class II processing cathepsin S protein [131]. Several of these modified rBCG vectors have demonstrated better protection against tuberculosis in animal models and are being moved into clinical trials. Because there is such a high degree of similarity between vector proteins and tuberculosis pathogens, this application is not a true test of the ability of rBCG to vector transgenic proteins. Much of the protection generated in these instances is against vector proteins, and not necessarily against the transgenic protein.

1.5 rBCG as a Vector

Genetic manipulation of BCG has made BCG a viable recombinant vector. The creation of an Escherichia coli/mycobacterial shuttle plasmid [132], and then the expression of foreign antigens within mycobacteria from this type of plasmid, demonstrated that rBCG can carry foreign DNA, produce a transgenic protein, and stimulate a transgene product-specific response in vivo [133, 134].

The recombinant shuttle plasmid used in this work is a derivative of the pMV261 plasmid first described by Stover et al. in 1991 [134]. This plasmid is a fusion of a multicopy E. coli origin of replication (oriE), a multicopy M. fortuitum plasmid pAL5000 origin of replication (oriM), aminoglycoside phosphotransferase antibiotic resistance selection gene (aph, kanR), and an expression cassette for transgenic protein expression using the Heat Shock Protein 60 (Hsp60) promoter. Replication in E. coli via the oriE allows easy genetic manipulation of the plasmid and the ability to prepare large quantities of it. Although the pAL5000 plasmid was originally isolated from M. fortuitum, the origin of mycobacterial replication is active within BCG and maintains the plasmid at a level of 6-8 copies per cell [134]. Transgene expression is driven by the Hsp60 promoter, a promoter which has been shown to drive high levels of transgene expression. Transgenic proteins are often fused to highly immunogenic mycobacterial export sequences, including the 19 kDa lipoprotein and the Ag85 secretion signal.

1.5.1 First Generation rBCG Vaccine Vectors

rBCG constructs have been created to vector antigens from Rotavirus, Human Immunodeficiency Virus (HIV), Borrelia burgdorferi, Streptococcus pneumoniae, pertussis toxin, tetanus toxin, measles virus, malaria, leishmania, respiratory syncytial virus, rabies, and Haemophilus influenzae. [135-139].

One of the first pathogens for which rBCG was used as a vector was B. burgdorferi, the etiological agent for Lyme disease. Studies examining the critical role of antibodies in the immune response against B. burgdorferi have indicated that the transfer of OspA-specific antibodies into SCID mice confers protection [140, 141]. Studies of a rBCG expressing the outer surface OspA lipoprotein from B. burgdorferi fused to the 19 kDa mycobacterial lipoprotein indicated that the rBCG-OspA strain generated high titer anti-OspA antibodies, and that these antibodies were effective in protecting mice from intradermal B. burgdorferi challenge [142]. It is important to note that investigators did not examine the T cell component of protection in this study. BCG is known to be an excellent inducer of cellular responses because of its intracellular lifestyle, given this sequestration away from the compartments that are normally accessed by B cells, it was surprising that a robust antibody response was generated against the transgenic protein. When mice were vaccinated intranasally, the high anti-OspA antibody titers were primarily IgG, with a modest IgA component [143]. The generation of such high antibody titers may be specific for the antigen used, as the increased titers were not as pronounced in response to S. pneumoniae antigens, although the degree of protection conferred by the rBCG immunization was equivalent [144, 145].

A large amount of work has gone into building rBCG strains vectoring HIV and SIV antigens. The first antigen cloned into rBCG after β-galactosidase was the HIV env gene. Numerous laboratories have cloned HIV gag, poi, nef and env into rBCG, as well as Simian Immunodeficiency Virus (SW) homologues of these genes [7, 9, 11, 146, 147]. When tested in small animal models, including mice and guinea pigs, these constructs have been shown to induce antibody responses, including in some cases neutralizing antibodies, against some HIV isolates [7, 148]. Cellular responses in small animal models against Gag and Env have also been induced by these rBCG vectors [149, 150].

Some first generation rBCG vaccines have been tested in nonhuman primate studies and human clinical trials with limited success. The aforementioned first generation rBCG-OspA vector displayed a good safety profile but failed to induce robust antibody titers in human vaccine trial volunteers [10].

The first generation rBCG strain vectoring HIV Env V3 induced antibody responses in rhesus monkeys capable of neutralizing SHIV strains carrying a homologous V3 loop, but these antibodies were unable to neutralize strains of HIV with a heterologous V3 loop. However, no investigation of the cellular anti-HIV response in these vaccinated monkeys was performed [151].

rBCG expressing the SW Gag protein was tested in a cynomolgus monkey model. These monkeys received a rBCG-SWV Gag prime and were boosted with a replication defective recombinant vaccinia virus (Dairen I) also expressing SIV Gag (DI-SW Gag). Weak Gag-specific ELISPOT responses were observed after primary inoculation with the rBCG-SIV Gag immunogen. A peak response of approximately 200 spot forming cells (SFC), determined by Gag-peptide ELISPOT assay, was observed 8 weeks after rBCG-SIV Gag priming. Boosting with DI-SIV Gag increased the Gag-peptide ELISPOT response to well over 1200 SFC per 10⁶ peripheral blood mononuclear cells (PBMC). Upon subsequent challenge with SHIV KS661c, no protection was afforded by rBCG-SIV Gag immunization alone, and only minimal protection was afforded by DI-SIV Gag immunization alone when assessed by plasma viral RNA copies/ml, CD4+ T cell count, and time to death. The prime/boost regimen was more effective; viral loads in two of three cynomolgus monkeys receiving the rBCG-SIV Gag prime/DI-SIV Gag boost combination were undetectable by 14 weeks following SHIV KS661c challenge [151].

A study examining rBCG-HIV Gag responses in baboons found no primary HIV Gag transgene product-specific T cell response in 5 of 6 animals tested using ELISPOT, and a response of under 250 SFC per 10⁶ PBMC in the sixth animal. This rBCG-HIV Gag construct primed for a Pr55(gag) virus-like particle boost response of up to 1000 SFC when tested by Gag-specific ELISPOT assay [152].

First generation rBCG strains vectoring SIV Gag have been created and tested for the generation of CD8+ T cell responses in a nonhuman primate study by our laboratory [9, 12]. A first generation rBCG-SIV Gag strain was shown to generate functional SIV Gag-specific CD8+ T cells, though no quantitation of the T cell response was done [12]. In a later study using a similar vector, two priming immunizations of rBCG-SIV Gag were administered 23 weeks apart, and a boost of rAd-5 SIV Gag was administered at week 43. While antivector PPD responses were very high following administration of rBCG-SIV Gag, transgene product-specific responses, as assessed by tetramer staining and IFN-γ ELISPOT, were disappointingly low. Low and inconsistent transgene-specific CD8+ T cell responses were observed after both priming and homolous boosting with rBCG-SIV Gag. It was only upon the subsequent heterologous boost with rAd5-SIV Gag that a noticeable difference was observed in the rBCG-SIV Gag-primed animals [9]. These data indicate that while first generation rBCG vaccines are capable of priming for increased transgene specific T cell responses upon heterologous boost, the T cell response following the rBCG prime is low or undetectable in nonhuman primate models.

1.5.2 Second Generation rBCG Vaccine Vectors

In nonhuman primate and clinical human studies using first generation rBCG vaccines as live recombinant vectors, we and others have observed disappointingly low transgene product-specific T cell responses, or low antibody responses. For this reason, a large amount of effort has gone into modifying BCG so that it generates increased immune responses, including more robust CD8+ T cell responses. Second generation rBCG vaccines constructed by genetically modifying the BCG strain have been developed in an attempt to address the low level of cellular immune responses formed against the transgenic protein, as compared to what would be expected from such a highly immunogenic intracellular pathogen.

Two second generation genetically modified rBCG vaccines have been created that attempt to redress the lack of phagosomal escape resulting from the RD1 attenuating deletion. As previously mentioned, RD 1-containing strains of pathogenic mycobacteria have access to the cytosol after internalization, and stimulate a larger CD8+ T cell response. This approach to vaccine vector creation includes the expression of a membrane-puncturing antigen.

1.5.2.1 Listeria Lysin-Expressing rBCG

The Kaufmann laboratory has created a rBCG expressing the listeria lysin (hly) gene (rBCG Δure::hly). Expression of the functional listeria lysin protein, a protein that punctures holes in the phagosomal membrane, did not allow egress of bacteria from the phagosome. However, it did allow antigen translocation from the phagosomal lumen to the cytosol, where it could be processed through traditional pathways for MHC class I presentation [153, 154]. No reports exist that document its use to vector antigens, but a very similar second generation rBCG construct created by the AERAS foundation has been used to vector foreign antigens.

1.5.2.2 Perfringolysin-Expressing rBCG

The AERAS foundation has created a second generation rBCG vector, AERAS 401, capable of destabilizing the phagosomal membrane through the expression of perfringolysin. In this rBCG vector the pfo.A gene from Clostridium perfringens was inserted into the BCG urease C gene, disrupting urease expression and allowing expression of perfringolysin (Pfo) by BCG [155]. The BCG gene encoding urease C prevents acidification of the phagosome and prevents MHC class II molecules loaded in that compartment from trafficking to the macrophage cell surface [156, 157]. Loss of urease C allows acidification of the vesicle, allowing Pfo to become active in this low pH environment. Pfo made by rBCG then destabilizes the phagosomal membrane. In addition, a single point mutation was included in the pfo.A gene (PfoA_{G137Q}) to make it less toxic to the host cell. Prior to this deletion, Pfo was potent enough to cause phagosomal membrane destabilization as well as cell membrane destabilization, killing the host APC [158].

The AERAS 401 strain of modified rBCG vectoring M. tuberculosis antigens (Ag85a, Ag85b, and TB10.4) induced strong antigen-specific immune responses in nonhuman primate studies [155], and was tested for its ability to vector a foreign transgenic protein, SIV Gag. AERAS 401 expressing the HIVA gag gene from an African clade A isolate of HIV-1 induced no antigen-specific immune response when administered alone in mice. In combination with an Ovine Atadenovirus-HIVA heterologous boost, however, strong Gag-specific cellular responses were generated. When tested in rhesus monkeys, the same observation was made. No Gag-specific cellular responses above background were observed to the primary immunization with the AERAS 401-HIVA strain when tested by pooled peptide IFN-γ ELISPOT assay, but upon boosting with Ovine Atadenovirus-HIVA, robust Gag-specific T cell responses were generated [159]. In a separate study, no primary Gag-specific responses were observed when neonatal macaques were immunized with the rBCG AERAS 401-HIVA strain, and subsequent heterologous boosting with recombinant modified vaccinia virus Ankara MVA.HIVA stimulated weak responses below 60 SFC per 10⁶ PBMC in all but one monkey [160]. It is important to note that no comparison between the priming ability of the second generation AERAS 401-HIVA strain and a first generation rBCG strain vectoring the HIVA antigen was performed.

1.6 Second Generation rBCG Creation by Screening Mutants

Primary transgene product-specific T cell responses have been low or undetectable following rBCG immunization in all nonhuman primate studies to date, and antibody responses against BCG-vectored Borrelia antigens were undetectable in human trials. Given the intracellular localization of BCG and the strong T cell responses and antibody responses generated against endogenous BCG antigens in the setting of infections, we found the low levels of MHC class I-restricted transgene product-specific immune responses surprising. A large amount of work has been done examining the mechanisms limiting MHC class II presentation of peptide associated with BCG while very little work has been done to study the mechanisms of BCG-mediated inhibition of MHC class I presentation of peptide. For these reasons, we set out to identify genes responsible for inhibiting MHC class I antigen presentation and disrupt these genes in order to create strains of rBCG. A method of creating a large library of BCG strains, each containing a random disruption in a single gene, has been developed and is described herein. An assay that can be used to screen these rBCG strains to identify those that generate increased MHC class I presentation in infected macrophages has also been developed and is described herein. The identification of novel strains would allow for the sequencing of the disrupted loci and identification of the genes responsible for inhibiting MHC class I presentation. Finally, we developed a site-directed method of gene disruption that can be used to generate novel rBCG vaccine vectors by disrupting the genes we identify herein as having the capacity to induce increased MHC class I-restricted T cell responses. This method of gene disruption can be applied to disrupt other genes as well, if desired.

1.6.1 Assay for Assessing MHC Class I Presentation

The generation of a T cell hybridoma specific for a model antigen presented in the context of MHC class I was described by Rock et al. [161]. A CD8+ T-T hybridoma specific for the highly immunogenic model antigen SIINFEKL, a peptide that is part of ovalbumin, was generated by the fusion of a SIINFEKL-specific T cell to the BW5147 cell line, which was already transfected with CD8 (BW5147-CD8). This novel T-T hybridoma responds to TCR stimulation by SIINFEKL presented in the context of MHC class I H-2K^{b} in conjunction with costimulation through the CD8 molecule. A T cell hybridoma is ideal for a large scale screen because it grows indefinitely in standard culture conditions, and allows for consistency between assays because it gives a standard fixed response. No in vitro restimulation of a hybridoma is necessary, which would introduce cyclical variations associated with the growth of primary T cells. The particular cell line used for fusion, BW5147, expresses cytokines in response to TCR/CD8 stimuli, and this provides a mechanism of measurement of the degree of T cell activation. One particular clone, the RF33.70 T-T hybridoma, has been used in studies assessing the presentation of the SIINFEKL epitope peptide for exploring proteolytic processing and cell types capable of presenting exogenous ovalbumin [162].

Researchers have also created a macrophage cell line through the transformation of the oncogenes myc and ras into bone marrow mononuclear cells [163]. The cell line A3.1A7 was shown to phagocytose exogenous antigens and present them in the context of MHC class I H-2K^{b} to the T-T cell hybridoma RF33.70 [162-164]. The presentation of exogenous SIINFEKL by A3.1A7 macrophages to RF33.70 T-T hybridoma cells has previously been used to assess the effect that M. tuberculosis infection has upon antigen presentation [165]. Production of the cytokine IL-2 by the hybridoma allows quantification of antigen presentation by infected macrophages.

1.6.2 BCG Mutagenesis

A library of rBCG strains can be generated through transposon mutagenesis. The mariner transposon is active in a variety of organisms including mycobacteria [166, 167]. At first transposon delivery made use of a two plasmid system: a suicide plasmid delivered the transposase enzyme and a second plasmid carried the transposon target sequence: short inverted repeats flanking a selectable kanamycin resistance marker. A single plasmid system was later developed, in which the transposase and transposon were encoded on the same plasmid. The transposase enzyme was encoded by DNA lying outside of the inverted repeats. Upon insertion of the transposon, the transposase gene is lost. This allows for single round integration and prevents successive excision and integration elsewhere in the genome of an organism.

1.6.3 Phage Delivery and Genetic Manipulation

Delivery of a transposon capable of functioning in mycobacteria can be accomplished via phage transduction. A large amount of mycobacterial genetic manipulation has been facilitated by the use of conditionally replicating, temperature sensitive phages. The D29 and TM4 phage have been modified so that they are capable of replication and propagation at 30° C., but they are unable to replicate at temperatures above 37° C. This technology can be harnessed by cloning a gene into the TM4 phage that becomes active within the mycobacterial cell at the nonpermissive temperature [168]. Phage technology is critical for mycobacterial research because it allows for the introduction of a genetic element into virtually all bacilli within a population, whereas other methods such as electroporation are less efficient. The mariner transposon has been cloned into a version of the TM4 conditionally replicating temperature-sensitive phage, creating a construct capable of delivering the mariner transposon efficiently to virtually all cells in a mycobacterial population [169].

1.6.4 Site-Directed Mutagenesis

Genes in BCG that are shown to decrease the host MHC class I antigen presentation can be deleted through allelic exchange. The delivery of an allelic exchange substrate to all cells within a mycobacterial population will be accomplished through specialized transduction using the temperature sensitive phage TM4. The use of specialized transduction avoids the high rates of illegitimate recombination that are associated with electroporation of allelic exchange substrates in slow growing mycobacteria [170-172]. Using an allelic exchange substrate with an insertion cassette containing hygromycin resistance as well as the levansucrase gene sacB allows for gene deletion and subsequent unmarking of these strains. Gamma delta resolvase target sites flank either side of the hygromycin resistance/sacB cassette, and counterselection by growth on sucrose-containing media after treatment with a gamma delta resolvase creates a novel strain of BCG containing a specific gene knockout, suitable for use as a vaccine construct [173, 174].

1.6.5 Application of Molecular Tools

Using the above listed tools, we generated information on genes that subvert the immune response and reduce the CD8+ T cell response generated to transgenic antigens vectored by BCG. This information was harnessed to create novel deletion strains of rBCG that increase CD8+ T cell responses against transgenic antigens. These BCG vectors, termed “second generation BCG vectors” herein, can be used as vaccine constructs for prophylactic or therapeutic vaccination (e.g., in the treatment or prevention of pathogenic infections, such as HIV, influenza, tuberculosis, and others described herein, cancer, allergy, autoimmune disease, and graft rejection) or for use as an adjuvant alone or in combination with other therapies. The BCG vectors of the invention can be used to deliver any peptide or protein of prophylactic or therapeutic value. For example, the BCG vectors of the invention can be used in the induction of an immune response (prophylactic or therapeutic) to any protein-based antigen.

The BCG vectors of the invention can each include a single epitope at one or more insertion sites (e.g., one or more sites in the genome of the BCG vector, in a plasmid, or within the site of one or more of the 15 genes identified herein as modulating CD8+ T cell responses). Alternatively, multiple epitopes can be inserted into the BCG vectors, either at a single site (e.g., as a polytope; if desired, the different epitopes can be separated by a flexible linker, such as a polyglycine, polyalanine, or polylysine stretch of amino acids), at different sites, or in any combination thereof. The different epitopes can be derived from a single species of pathogen, or can be derived from different species and/or different genera. The BCG vectors can include multiple peptides, for example, multiple copies of peptides as listed herein or known in the art for promoting an immune response, or combinations of peptides such as those listed herein or known in the art for promoting an immune response.

Antigens that can be used in the BCG vectors of the invention can be derived from, for example, infectious agents such as viruses, bacteria, fungi, and parasites.

### II. Identification of BCG Genes that Suppress MHC Class I Restricted CD8+ T Cell Responses

### Abstract

Mycobacterium bovis BCG is a potent stimulator of the cellular immune response and can be used as a recombinant vector vaccine. However, the bacterium contains genes that reduce the host's CD8+ T cell response. We have developed and implemented a screen to identify BCG genes that decrease MHC class I presentation following vaccination. In screening 3290 transposon mutant strains of recombinant BCG (rBCG), we identified 122 strains that generated greater in vitro MHC class I presentation of a transgenic protein than the unmutated rBCG parental strain. When tested in vivo in a mouse model, 37 of these select rBCG transposon strains generated primary transgene product-specific CD8+ T cell responses that were greater than responses generated by the parental strain. Finally, when boosted with a heterologous vaccine construct, 17 of these strains primed for secondary transgene product-specific CD8+ T cell responses that were greater than the responses primed for by the parental strain. Transposon disruptions were sequenced in two strains and identified to disrupted the gene BCG_{—}1790 (Rv1751) and the operon containing the genes echA18 and amiD. Strains containing these disruptions do not exhibit increased growth in vivo, suggesting that increased pathogenicity is not the cause of the increased immunogenicity. These findings demonstrate a method of identifying novel mycobacterial genes that modulate the immune response and can be used in the creation of a more immunogenic rBCG vector.

### Introduction

M. bovis Bacillus Calmette-Guerin can be used as a vaccine vector to induce very strong cellular immune responses in mammalian species (e.g., humans, monkeys, mice, dogs, and cats). The technology to manipulate mycobacteria genetically through the use of bacteriophages and E. coli/mycobacteria shuttle plasmids has facilitated the expression of transgenic antigens in BCG. A number of first generation rBCG vaccines have been generated expressing antigens from a variety of pathogens including Borrelia burgdorferi and HIV-1. However, all of these rBCG constructs have had disappointing immunogenicity [9-11].

Mycobacterial infections lead to the generation of very strong CD4+ T cells responses during acute infection and strong CD8+ T cell responses during chronic infection. During the course of infection, BCG has been shown to reside in the phagocytic compartment of macrophages where it can reproduce and produce proteins. These proteins gain access to both the MHC class I and MHC class II processing pathways [65]. The majority of T cell responses generated during mycobacterial infections are focused on epitopes of secreted or cell surface-associated proteins. In some pathogenic mycobacterial infections, CD8+ T cell responses specific for secreted proteins comprise as much as 40% of all lung CD8+ T cells as quantitated by tetramer staining [81].

However, despite the generation of strong anti-vector T cell responses following rBCG administration, there are a number of mechanisms by which BCG limits antigen presentation. Mycobacteria thus appear to have evolved mechanisms to avoid MHC class I- and class II-restricted immune responses. Elimination of these mechanisms by the methods described herein allow for the generation of BCG vectors that exhibit even greater T cell responses than that observed using unmodified BCG.

While strong anti-vector T cell responses are generated following rBCG administration, responses of a comparable magnitude against transgenic antigens have not been observed. In murine studies, we and others have observed low rBCG-induced primary transgene product-specific CD8+ T cell responses. We have also observed undetectable primary transgene product-specific CD8+ T cell responses in nonhuman primates vaccinated with rBCG [9].

In the present study, we have identified a number of strains of BCG with disruptions of genes encoding proteins that act in pathways that limit transgene product-specific CD8+ T cell responses. We established a large scale, high throughput screen capable of quantifying antigen presentation by mycobacteria-infected macrophages and used this screen to identify 17 strains of rBCG with enhanced transgene product-specific immunogenicity.

### Materials and Methods

Mice. Age-matched adult C57Bl/6 mice were obtained from Jackson Laboratories (Bar Harbor, Me.). All mice were maintained in the BIDMC Animal Research Facilities and used in accordance with protocols approved by the Institutional Animal Care and Use Committees (IACUC) of BIDMC, Harvard Institutes of Medicine, and Harvard Medical School.

Creation of parental BCG strain. A 15 ml culture of BCG Danish 1331 was grown to an OD=1, pelleted at 3000 rpm, and washed three times with a solution of 10% glycerol. The pellet was resuspended in 200 μl of 10% glycerol, mixed with 200 ng DNA of the plasmid pMV261-19 kDaSIINFEKL, and incubated for 20 minutes. Cells were then transformed by electroporation (2.5 kV, 25 mF, 1000 ohms). Electroporated cells were incubated in 7H9 media overnight and then plated on 7H10 plates with 20 μg/ml kanamycin. Three weeks later, colonies were selected and grown to an optical density of 1. Western blot analysis on 1.25×10⁶ colony forming units (CFU) of the rBCG was performed using polyclonal rabbit-anti-ovalbumin serum (courtesy of Steven Porcelli, Albert Einstein College of Medicine) and the Roche-BM chemiluminescent anti-rabbit detection kit to assess protein expression from the pMV261-19 kDaSIINFEKL plasmid.

Transposon library synthesis. One hundred ml BCG Danish-pMV261-19 kDaSIINFEKL was grown to an optical density of 1, pelleted at 3000 rpm, and washed twice with buffer MP (50 mM tris, 150 mM NaCl, 10 mM MgCl₂, 2 mM CaCl₂). Pelleted cells were then resuspended in 3 ml of buffer MP containing 10¹⁰ PFU of the transposon-containing phage pCS9Hygro1Mar, incubated for 24 hours, and plated on 10 plates of 7H10 containing 100 μg/ml hygromycin and 20 μg/ml kanamycin. 5000 colonies were selected and each grown in 10 ml 7H9 with 20 μg/ml kanamycin in a 15 ml conical tube maintained in a roller at 37° C.

MHC class I presentation assay. Cells from the H-2K^{b} macrophage cell line A3.1A7 were washed with RPMI-10% FCS, resuspended at 5×10⁵ cells/ml, and 100 μl was aliquoted in each well of a 96 well plate (5×10⁴ cells/well). Cells were activated with 250 U/ml IFN-β for 2 hours at 37° C. Mycobacterial strains expressing the epitope SIINFEKL were washed with PBS-0.02% tween 20, resuspended at 4×10⁷ CFU/ml in RPMI without antibiotics and 50 μl (2×10⁶ CFU) was added to each well (MOI of 40). RF33.70 T cells (courtesy of Dr. Kenneth Rock, University of Massachusetts Medical School) were resuspended in RPMI-10% FCS at a concentration of 2×10⁶, and 50 μl was added to each well (1×10⁵ cells/well). Ninety six well plates were incubated at 37° C. for 24 hours and then frozen at −20° C. until IL-2 production was assayed by ELISA. One hundred μl of supernatant was assayed for IL-2 production using the Invitrogen IL-2 ELISA kit. IL-2 levels were determined by comparison to the standard curve calculated using an IL-2 protein control.

BCG burden assessment. BCG strains were grown to an OD of 1. One hundred million CFU were isolated, washed and then resuspended in 1 ml of PBS-tween 20 0.02%. One hundred μl (10⁷ CFU) was injected IV by tail vein injection into C57Bl/6 mice. At the indicated time points, mice were sacrificed, and spleens, livers, and lungs were isolated into 2 ml RPMI-10% FCS. Organs were massed, homogenized, and the homogenates were plated at serial dilutions of 10⁻¹, 10⁻³, 10⁻⁵ on 7H10 plates containing kanamycin at 20 μg/ml. Two weeks later CFU were enumerated and bacterial burden per organ was calculated.

AERAS 401-SIINFEKL construction. AERAS 401, a recombinant strain of BCG lacking the Urease C gene and containing the perfringolysin O gene (PfoA_{G137Q}) was obtained from the AERAS foundation. Confirmation that the strain was AERAS 401 was performed using primers included in patent application Ser. No. 11/755,936, filed May 31, 2007 (ACGGCTACCGTCTGGACAT (SEQ ID NO: 2) and CGATGGCTTCTTCGATGC (SEQ ID NO: 3)). The plasmid pMV261-19 kdaSIINFEKL was transformed into AERAS 401. Expression from AERAS 401 containing the 19 kdaSIINFEKL plasmid was assessed by Western blot using an anti-SIINFEKL rabbit antiserum.

Identification of transposon location. Nine ml of each transposon mutant was grown to an optical density (OD) of 0.8. One ml of 10% glycine (wt/vol) was added to 9 ml BCG culture for 24 hrs. Cultures were then pelleted, incubated with Lysis Buffer for 1 hr at 65° C. and genomic DNA was isolated using the DNeasy Qiagen kit. Genomic DNA was digested for 2 hrs with BssHII, and circularized by ligation with T4 DNA ligase for 1 hr to make plasmids bearing the pir-dependent origin of replication. These plasmids were subsequently transformed into pir2000 electrocompetent cells. Cells were plated on LB agar plates with hygromycin at a concentration of 100 μg/ml. Pir2000 cells containing viable plasmids grew into colonies, and plasmid DNA was isolated using the Qiagen Miniprep Spin Kit. Plasmids were sequenced using the primers “LIR” and “RIR” that bind internally to the LIR and RIR of the transposon. Sequences were aligned with the BCG Pasteur sequence AM408590 using NCBI Blast to identify the genes surrounding the site of transposon disruption, allowing the identification of the disrupted pair of nucleotides.

Complementation. BCG strains K14 and AZ11 were chosen for complementation. PCR was performed using primers (AACCAAGCTTTCGGCGATTGTGATGAGG; SEQ ID NO: 4) and (AACCAAGCTTCTATCCGGGCTATACCCAAGAC; SEQ ID NO: 5) to amplify the K14 locus and primers (AACCAAGCTTTGTTTGCGCTGGACAGTCCC; SEQ ID NO: 6) and (AACCAAGCTTAACGCGTCCTCCCTTGATGG; SEQ ID NO: 7) to amplify the AZ11 locus. The primers created a PCR product with a HindIII site at both ends. The product was purified and digested with the HindIII enzyme. The plasmid pYUB 1141 was digested with the single cutter enzyme HindIII and then purified. Digested PCR product was ligated into the pYUB 1141 backbone, and the ligation product was transformed into DH5a E. coli and selected on LB-agar plates containing 100 μg/ml apramycin. Plasmid was isolated from E. coli colonies. Sequencing and digestion were used to confirm successful cloning and determine the orientation of the ligation product within pYUB 1141. The K14 fragment was cloned in the “forward” orientation, and the AZ11 fragment was cloned in the “reverse” orientation with regard to the Hsp60 promoter. Successfully cloned plasmids were transformed into K14 and AZ11 transposon strains, respectively, and selected on 7H10 plates containing 20 μg/ml kanamycin and 30 g/ml apramycin. Successful complementation was assessed by PCR, using primers K14F (GATGCCATCAATGGCCAGGAG; SEQ ID NO: 8), K14R (ACGAAAGTCGCGCAGGAAG; SEQ ID NO: 9), AZ11F3 (GTTGACCACGATGCCGATCC; SEQ ID NO: 10), AZ11R3 (GCATGACGAAGGCGTAGCTC; SEQ ID NO: 11).

Colony PCR. Forty nine p1 of a stock PCR mixture containing the BD Advantage Taq polymerase, dNTP, buffer and primers to amplify the gene of interest were aliquoted into PCR tubes. A small but visible amount of bacterial colony was taken off the plate (estimated volume 11) and added to the appropriate tube. For culture PCR, 1 μl of BCG culture at an OD>0.5 was added to the PCR reaction. Samples were denatured at 95° C. for 10 minutes prior to PCR. Fifteen μl of a 50 μl PCR reaction was run in each lane.

Listeria challenge. Eight weeks after C57Bl/6 mice were primed with rBCG strains expressing SIINFEKL, mice were given 5×10⁴ CFU IV of erythromycin-resistant Listeria monocytogenes (H. Shen, University of Pennsylvania School of Medicine) expressing ovalbumin (rLM-OVA). On day 3, spleens were extracted, homogenized, and plated at serial dilutions on Brain-Heart Infusion agar plates containing erythromycin.

Immunization protocol. One hundred million CFU from an actively growing log phase BCG culture (between OD=0.6 and 0.9) was isolated, pelleted and resuspended in 1 ml PBS-tween 20 0.2%. One hundred μl (10⁷ CFU) was injected IV into each of 4-8 C57Bl/6 mice per group. Twenty five μg of plasmid DNA was injected into each hind leg quadriceps muscle for a total of 50 μg DNA/mouse. One million PFU rAd5-SIINFEKL or 10⁷ PFU rAd5-SIV Gag was injected IM into the hind leg quadriceps of mice.

Tetramer staining. Seven to 14 days after vaccination, 100 μl of peripheral blood was collected from each mouse into RPMI via cheek bleeds, ACK treated to remove red blood cells, and stained using an H-2K^{b}-SIINFEKL-PE and anti-CD8-PerCP-Cy5.5 antibodies. Results are displayed as the CD8+ T cells that stained positive by tetramer as a percent of the total number of peripheral blood CD8+ T cells.

Analysis. Statistical analyses were performed using a one-way ANOVA on PRISM5 software. P<0.05: *, P<0.01: **, P<0.001: ***. Densitometry calculations were performed using ImageJ software.

### Results

Assay for MHC Class I Presentation

An assay first was established for quantifying MHC class I presentation of a transgene epitope expressed in a macrophage population by recombinant mycobacteria. The ability of the A3.1A7 macrophage cell line to present the peptide SIINFEKL in association with the H-2K^{b} MHC class I molecule has been described [163].

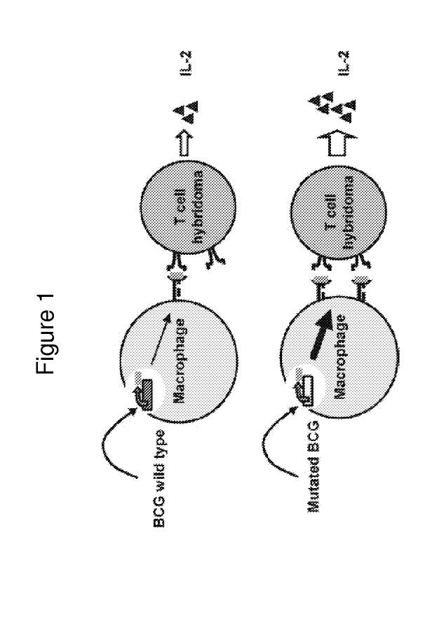

As previously shown, when this antigen presenting cell (APC) line expressing the H-2K^{b} molecule is pulsed with the peptide SIINFEKL and then incubated with the T cell hybridoma RF33.70, IL-2 production by the RF33.70 T cell hybridoma occurs at levels that are proportional to the number of H-2K^{b}-SIINFEKL complexes on the surface of the pulsed APCs (FIG. 1) [162]. We were able to measure IL-2 production from the RF33.70 T cell hybridoma by sandwich ELISA, and use that value as a measure of SIINFEKL presentation by the APC line.

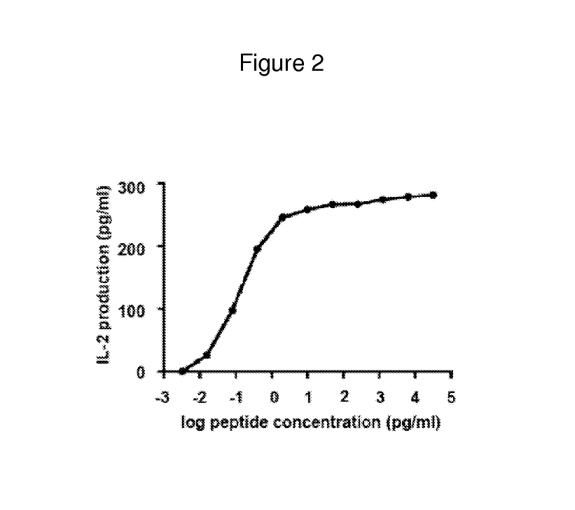

The responsiveness of this assay was tested to a range of SIINFEKL concentrations (FIG. 2). A detectable IL-2 response was generated by the RF33.70 T cell hybridoma to A3.1A7 cells pulsed with under 0.1 pg/ml of SIINFEKL peptide, and there was a linear increase in the IL-2 response between concentrations of 0.1 pg/ml and 1 pg/ml SIINFEKL.

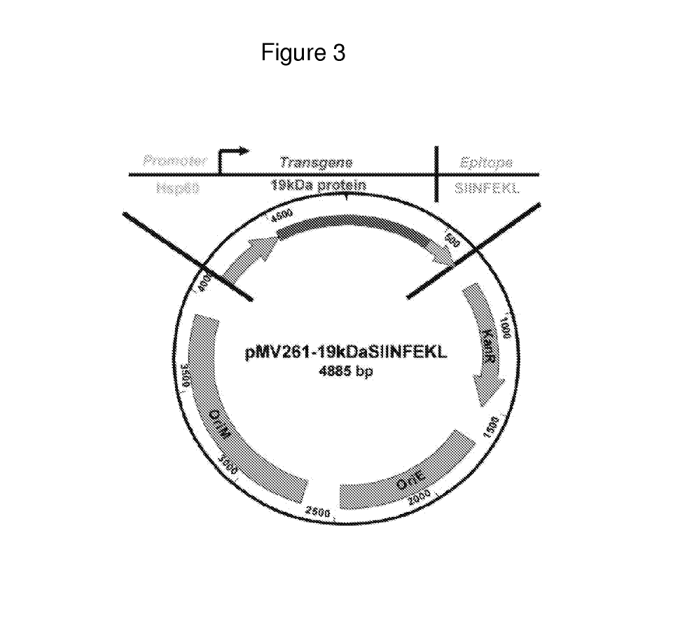

We then sought to determine if this assay was capable of detecting SIINFEKL presentation when the SIINFEKL epitope was expressed by recombinant mycobacteria. We transformed a number of E. coli/mycobacteria shuttle plasmids containing the SIINFEKL epitope fused to different immunogenic proteins into the fast-growing strain M. smegmatis mc²155 and tested the new recombinant M. smegmatis strains following ingestion by the A3.1A7 APC line for their ability to induce in vitro IL-2 production by the T cell hybridoma RF33.70. Of the strains tested, the most IL-2 production was generated in response to the M. smegmatis strain expressing the SIINFEKL epitope fused to the full length 19 kDa lipoprotein, Rv3763. This M. smegmatis strain was transfected by a multicopy episomal mycobacteria/E. coli shuttle plasmid that carries a kanamycin resistance gene and contains the 19 kDa lipoprotein Rv3763 with a SIINFEKL epitope fused to the C terminus, all under control of the Hsp60 promoter (FIG. 3).

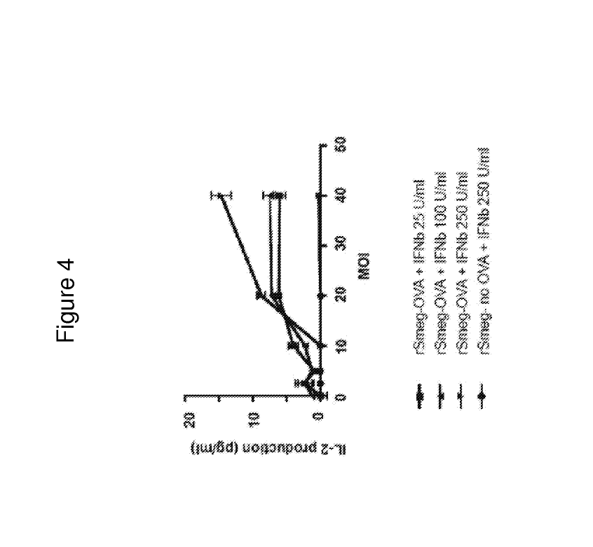

The sensitivity of this assay for detecting SIINFEKL presentation in association with MHC class I following ingestion of the recombinant M. smegmatis by the APC line was tested over a range of multiplicities of infection (MOI) and following activation by increasing concentrations of the cytokine Interferon beta (IFN-β) (FIG. 4). The addition of IFN-β to the APC was critical to the production of IL-2 by infected APC. In APC activated with IFN-β, limited production of IL-2 was detected at MOI as low as 2.5, and IL-2 production increased as the MOI increased. Increasing IL-2 production that corresponded with increased MOI was more pronounced at the highest concentration of IFN-β, 250 U/ml. The most IL-2 production, approximately 15 pg/ml, was detected in APC infected at an MOI of 40 in combination with 250 U/ml of IFN-β. The level of IL-2 production in response to mycobacterially vectored SIINFEKL (0-15 pg/ml) was lower than the level of IL-2 production generated in response to free SIINFEKL peptide (0-275 pg/ml). Based on these data, we concluded that the assay would be most sensitive using an MOI of 40 and stimulating the APC with 250 U/ml IFN-β.

Using this in vitro assay as a starting place for quantifying MHC class I presentation of SIINFEKL by infected macrophages, we then developed a high throughput 96-well plate assay that could be used to screen a large rBCG transposon mutant library and determine which mutant rBCG strains generated increased presentation of the transgene product. Both RF33.70 and A3.1A7 hybridoma cells were grown in large quantities. Large numbers of BCG strains were grown in rollers in incubators and tested in triplicate.

Creation of rBCG-SIINFEKL Transposon Mutant Library

Because the highest IL-2 response in the assay was observed in M. smegmatis expressing the 19 kDaSIINFEKL protein, we chose to create a parental strain of rBCG using the pMV261-19 kDaSIINFEKL plasmid and we made a mutant library from that strain. A library of several thousand transposon mutant strains was created using the mariner transposon to disrupt genes in the parental rBCG. The parental rBCG strain was transduced with a phage carrying pCS9Hygro1-MAR, which encodes a transposon that inserts itself into the BCG genome. This transposon requires only a TA dinucleotide and therefore has minimal site specificity [166]. In fact, we sequenced 17 random rBCG transposon mutants and did not observe any preference for insertion in a specific region in the BCG genome.

The mariner transposon inserts a hygromycin cassette of approximately 2200 bp, disrupting any gene it bisects and possibly having polar effects on downstream genes in operons. The gene encoding the transposase enzyme is not inserted into the BCG genome and is therefore lost, preventing successive hops around the genome. Tranposon mutagenesis did not affect SIINFEKL expression from the rBCG.

In Vitro Screen of rBCG Mutant Library

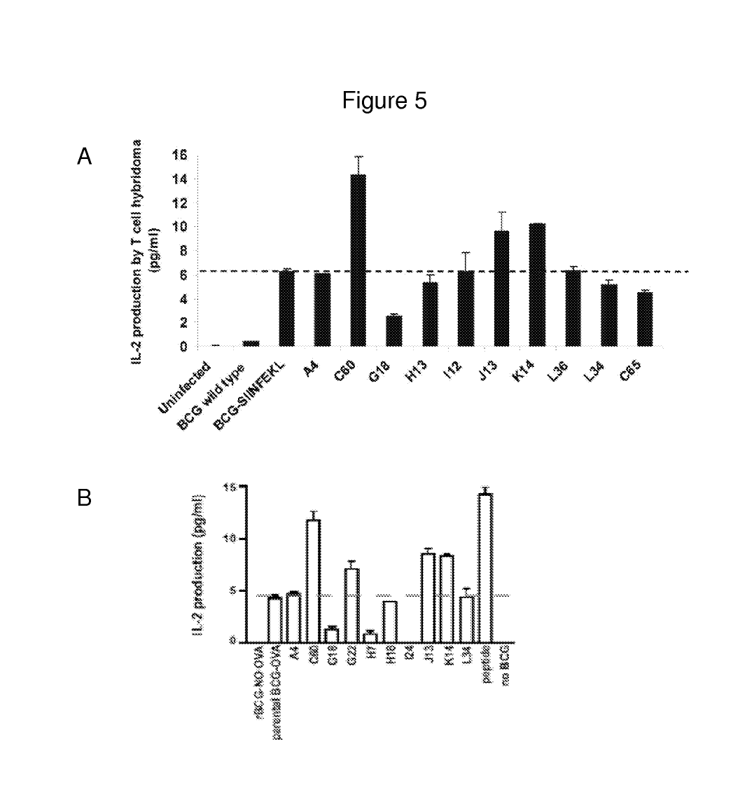

Three thousand two hundred ninety individual transposon mutant strains were tested in triplicate in the peptide/MHC class I presentation assay. Transposon mutant strains that generated an IL-2 response higher than the level generated by the unmutated parental strain were tested a second time to confirm the finding of the elevated response. The data shown in FIGS. 5A and 5B are representative of the data obtained from a typical assay. The parental rBCG-SIINFEKL-infected APCs elicited approximately 6 pg/ml of IL-2. Most mutant strains tested elicited a level of IL-2 comparable to that elicited by the parental strain, and some mutant strains elicited a very low level of IL-2 production. Other mutant strains, such as C60, J13, and K14, elicited a markedly higher level of IL-2 production compared to the parental strain and were therefore selected for in vivo testing.

In Vivo Screening of Selected rBCG Mutant Strains

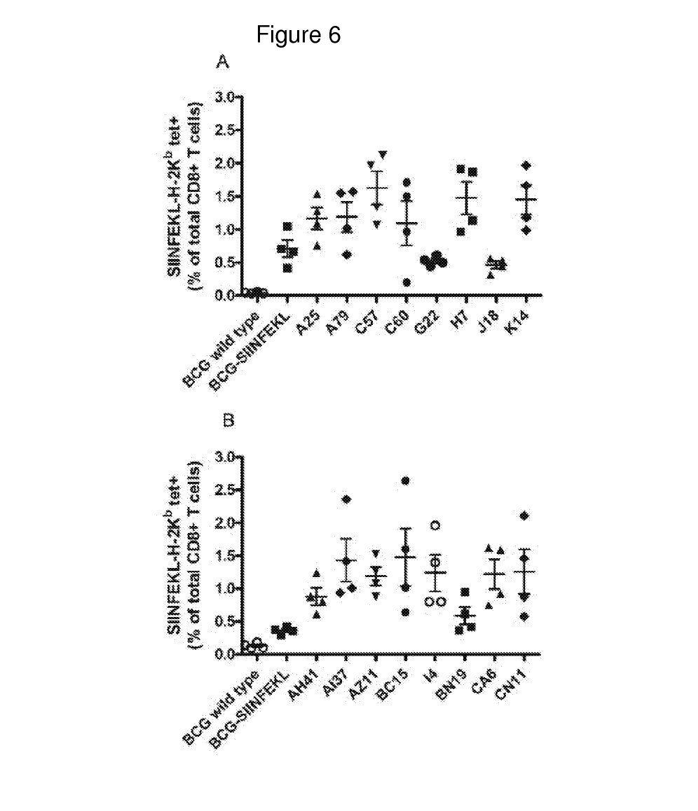

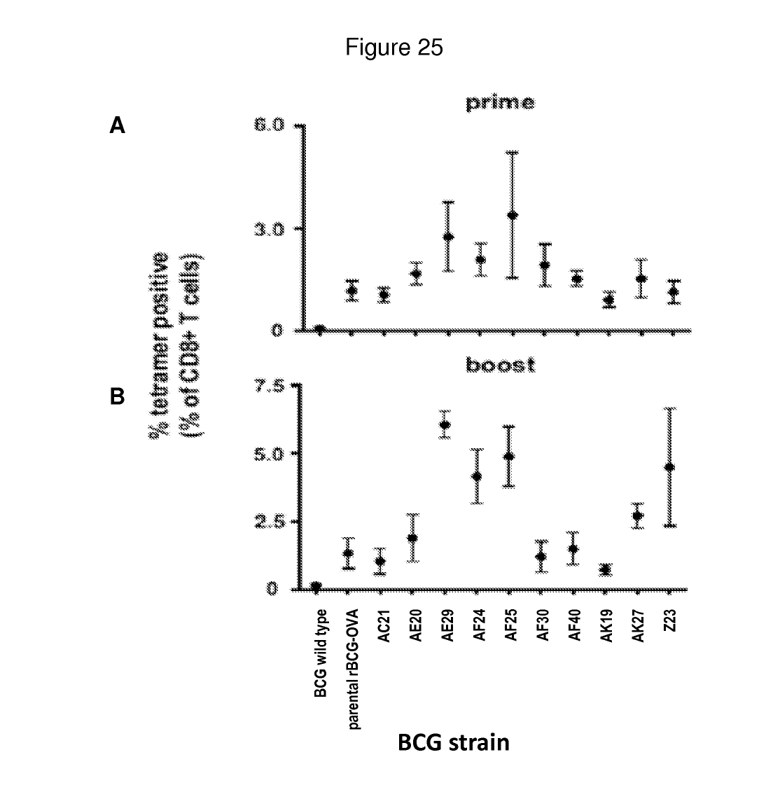

One hundred twenty two transposon mutant rBCG strains were selected for in vivo testing, representing 3.7% of the total number of strains tested in vitro. Seventy six of these 122 strains were tested for their ability to generate H-2K^{b}: SIINFEKL tetramer responses in vivo. Groups of 4 to 8 C57Bl/6 (h-2^{b}) mice were inoculated intravenously with 10⁷ CFU of the selected strain, and the SIINFEKL-specific CD8+ T cell response was assessed by H-2K^{b}-SIINFEKL tetramer staining 7, 14, and 21 days later. The parental strain consistently generated mean peak tetramer responses that ranged from 0.4% to 0.8% of total peripheral blood CD8⁺ T cells. Nearly half of the strains selected for in vivo immunogenicity studies ( 37/76, 50%) elicited greater transgene product-specific CD8⁺ T cell responses than those elicited by the parental strain. Representative data from an assay in which mutant strains were tested for their ability to generate primary in vivo tetramer responses that were greater than the responses generated by the parental strain are shown in FIGS. 6A and 25A.

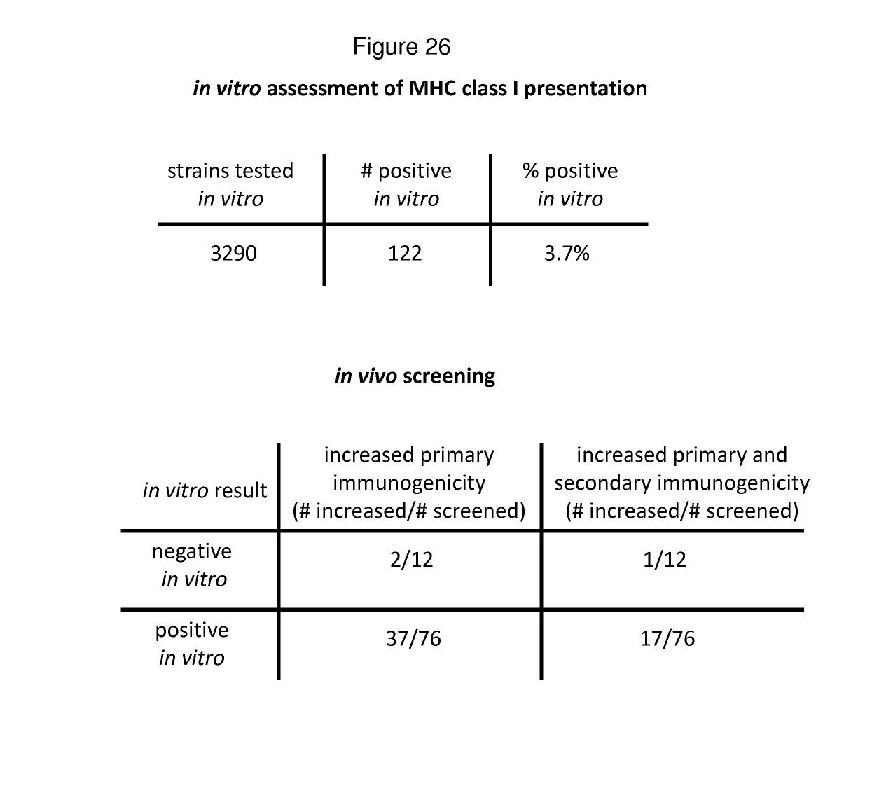

After a minimum of several months, all immunized mice were boosted with a suboptimal dose of 10⁶ viral particles (vp) of rAd5-SIINFEKL to provide the greatest discrimination between these various mutant strains of rBCG for their ability to prime in a rBCG/rAd5 vaccine regimen. Upon boosting with rAd-STINFEKL, 17 of the novel strains ( 17/76, 20%) that induced increased primary responses also primed for increased secondary responses relative to the unmutated parental strain of rBCG. FIGS. 6B and 25B show representative data following boosting of one cohort of mice. Ten days after boosting with rAd-SIINFEKL, mice primed with the parental rBCG strain had a mean tetramer response of 1.3%, while a number of the mutant strains primed for boosted responses of 4-7%, significantly greater than the response primed by the parental strain. In vitro and in vivo screening results for all 3290 strains tested are summarized in FIG. 26. Forty nine percent (49%) of the mutant strains selected by in vitro screening elicited greater responses than those elicited by the parental strain, while only 2 of 12 unselected strains generated increased tetramer responses when tested in vivo. Thus, the in vitro screen enriched the pool of mutants with increased immunogenicity for CD8⁺ T cell responses (Table 1).

TABLE 1
Summary of in vitro presentation and in vivo immunogenicity screening
of rBCG transposon mutant strains. Absolute numbers and percentages
are given for the number of mutant strains identified in each
part of the screen that generated responses greater than the
responses generated by the parental rBCG strain.
i. In vitro assessment of MHC class I presentation

| | Increased |
|---|---|
| | MHC class I presentation |
| No. strains tested | in vitro |
| 3290 | 122 (3.7%) |

ii. In vivo assessment of SIINFEKL-specific CD8+ T cells

| | | Increased |
|---|---|---|
| | | MHC class I presentation |
| | No. strains tested | in vivo |
| Not generating | 12 | 2 (16.7%) |
| increased | | |
| in vitro responses | | |
| Generating | 76 | 37 (49%) |
| increased | | |
| in vitro responses | | |

Boost of rBCG-Primed Mice with rAd5-SIINFEKL

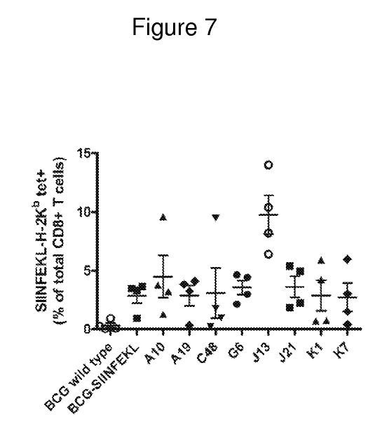

Selected rBCG strains that elicited increased CD8+ T cell responses to SIINFEKL were assessed with a suboptimal boost of 10⁶ particles of rAd5-SIINFEKL for their ability to prime in a rBCG/rAd5 vaccine regimen. This suboptimal dose of rAd5-SIINFEKL was chosen to provide the greatest discrimination between the priming ability of these various mutant strains of rBCG. FIG. 7 shows representative data from one of these experiments. Ten days after boosting with rAd-SIINFEKl, mice primed with the parental rBCG strain had a tetramer response of approximately 3%, while the mutant strains primed for boosted responses of 5-15%, significantly greater than the parental strain.

Comparison of Novel Mutant rBCG Strains with an Existing Modified BCG Vector

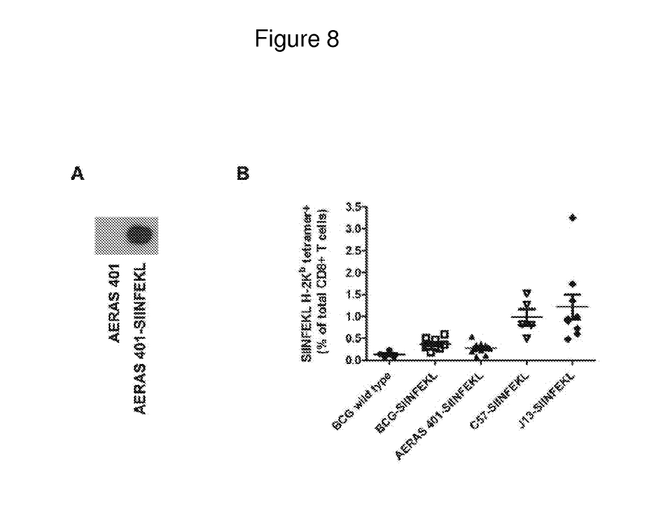

We next compared selected mutants generated in this study to another modified rBCG that is currently under evaluation in human trials for their ability to vector transgenic proteins. We chose the rBCG strain AERAS 401, a BCG strain modified by the insertion of the perfringolysin gene into the BCG urease gene, and transformed AERAS 401 with the plasmid pMV261-19 kDaSIINFEKL [155]. SIINFEKL expression from AERAS 401 was readily demonstrated (FIG. 8A). Transposon mutant strains C57 and J13 expressing SIINFEKL were then compared to AERAS 401-SIINFEKL for their ability to stimulate an in vivo primary SIINFEKL-specific CD8+ T cell response (FIG. 8B). AERAS 401-SIINFEKL stimulated a SIINFEKL-specific CD8+ T cell response (0.23%) compared to that of BCG Danish vectoring SIINFEKL (0.37%). However, both transposon mutants, C57 and J13, stimulated SIINFEKL-specific CD8+ T cell responses approximately 3-fold higher than that of BCG Danish-SIINFEKL (0.98% and 1.2%, respectively). Therefore, these novel transposon mutant strains are more immunogenic than a modified BCG vector that is currently in human clinical trials.

In Vivo Clearance of Novel BCG Strains

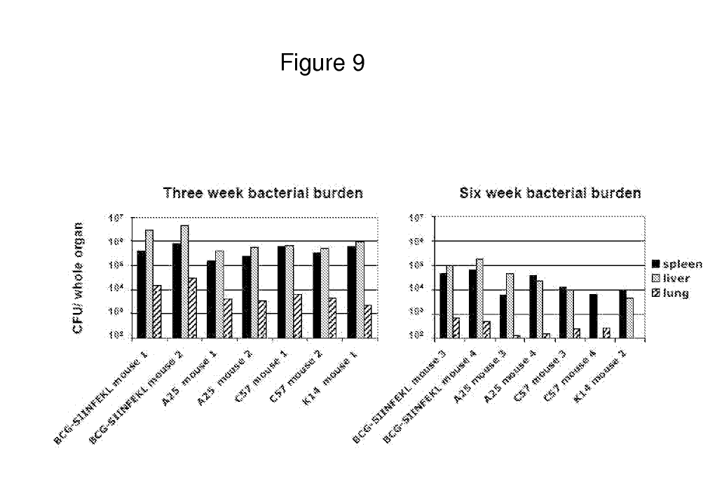

The immunogenicity of BCG strains may be associated with the virulence of the strains. If the selected transposon mutant strains of BCG have increased immunogenicity as a consequence of increased pathogenicity, they would not be viable candidates for clinical development. Mutations may cause the bacteria to grow more rapidly in vivo, leading to higher BCG burdens and increased immunogenicity. We examined three mutant BCG strains (A25, C57, and K14) for their in vivo growth in the spleen, liver, and lungs. Organs were harvested at 3 and 6 months after inoculation and colony counts were performed over a range of serial dilutions (FIG. 9). In all organs, at both time points, there were lower numbers of A25, C57, and K14 than parental colonies, indicating that the increased immunogenicity of these strains is not caused by an increase in their in vivo growth kinetics.

Complementation of Selected rBCG Strains

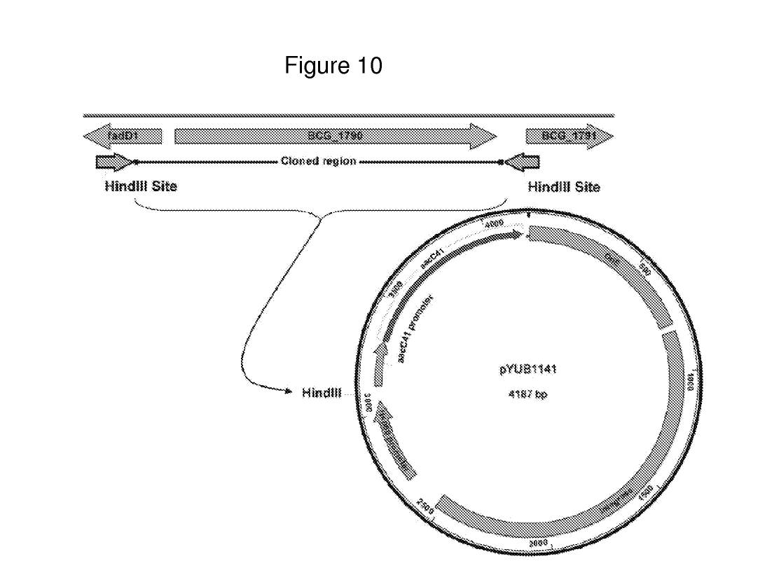

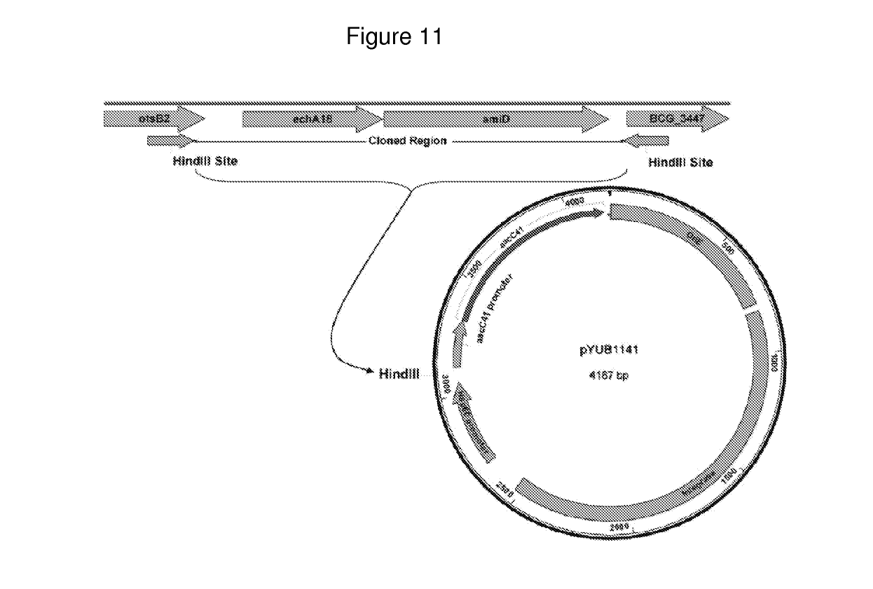

To confirm that the phenotype of increased immunogenicity associated with the selected mutants was a consequence of transposon disruption of single genes, we complemented these mutants with functional copies of the disrupted genes and assessed their phenotype. Strains K14 (ICO E) and AZ11 (ICO N) were chosen for complementation because the small size of the disrupted genes facilitated the cloning process. Since the K14 transposon disrupted one gene, BCG_{—}1790, this gene and its endogenous promoter were cloned into the integrating apramycin-resistant expression plasmid pYUB 1141 and then transformed into the K14 strain (FIG. 10). The disrupted operon of AZ11 containing two genes, echA18 and amiD and its endogenous promoter, were cloned into pYUB 1141 and then transformed into AZ11 (FIG. 11).

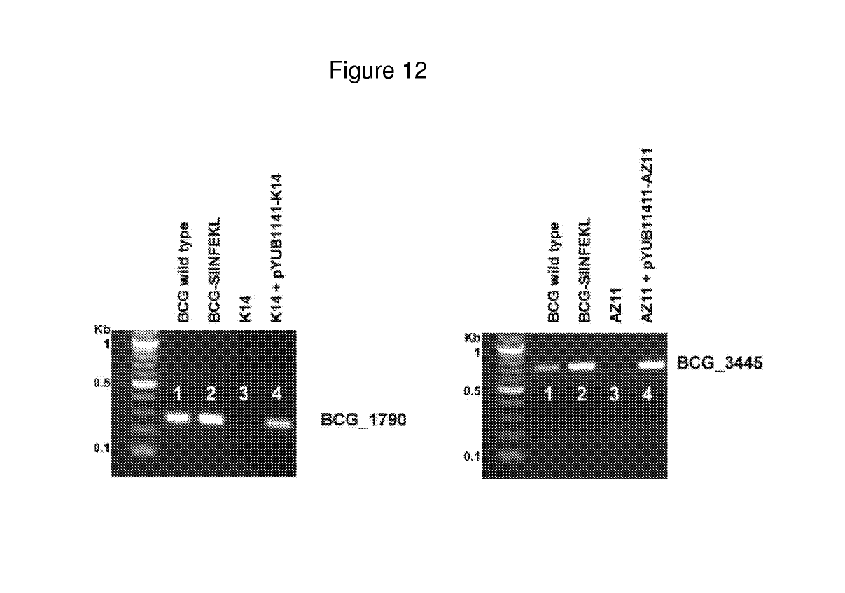

PCR analyses showed that complementation by pYUB 1141-K14 returned a functional BGC_{—}1790 gene to the K14 strain (FIG. 12A), and complementation by pYUB1141-AZ11 returned a functional BCG_{—}3445 gene to the AZ11 strain (FIG. 12B).

Tetramer Responses to the Complemented rBCG Strains

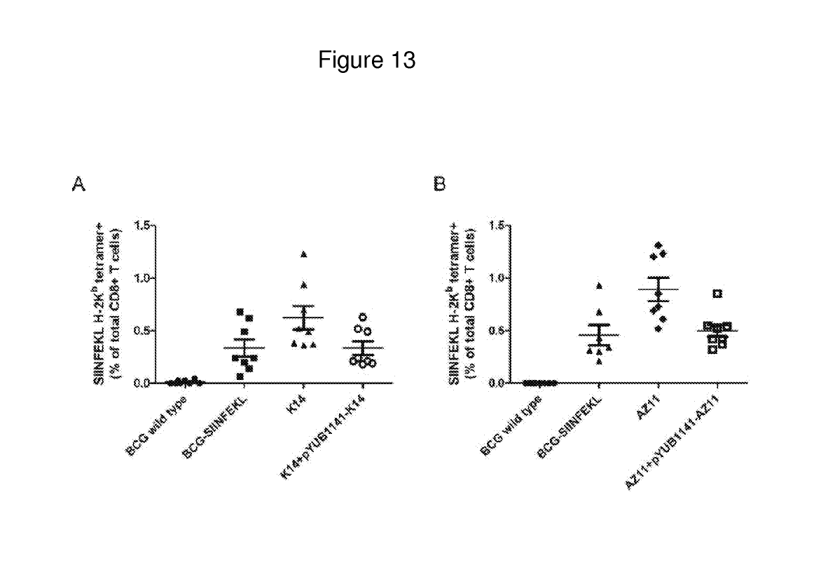

The complemented K14 and AZ11 strains were then compared to the K14 and AZ11 strains for their ability to elicit tetramer responses in mice. The K14 strain generated a response of 0.62%, approximately twice the response generated by the parental BCG-SIINFEKL strain (0.33%), and the strain K14 complemented with the BCG_{—}1790 gene generated a reduced tetramer response of 0.33%, comparable to that generated by the wild type rBCG strain (FIG. 13A). Similarly, the AZ 11 strain generated a tetramer response of 0.89%, approximately twice the response generated by the parental BCG-SIINFEKL strain (0.46%), and the AZ11 strain complemented with the BCG_{—}3455 gene generated a reduced tetramer response of 0.5%, comparable to the response to parental BCG-SIINFEKL strain (FIG. 13B). Therefore, for both K14 and AZ11, the phenotype of increased immunogenicity was converted to the wild type phenotype when the strains were complemented with a functional copy of their respective disrupted genes. This finding formally demonstrated that the disruption of the two-gene operon containing echA18 and amiD was responsible for the enhanced immunogenicity of the ICO N rBCG mutant strain.

### Discussion

BCG is a viable vaccine vector for a number of infectious agents by virtue of the ease of expressing foreign transgenes in recombinant BCG constructs. However, BCG retains immunosuppressive properties that limit its potential utility as vaccine immunogen. By developing a high-throughput in vitro screen for assessing MHC class I peptide presentation and applying this screen to a rBCG transposon mutant library, we have identified rBCG strains with disruptions in a number of immunosuppressive pathways. This study represents the first step toward identifying these immunosuppressive pathways and deleting them to develop a more immunogenic rBCG vaccine vector.

Using a novel two-tiered loss-of-function screen, we have demonstrated the ability to identify mutant strains of rBCG that generate better CD8+ T cell responses than the parental BCG organism. The primary in vitro screen, a high-throughput assay that was an adaptation of an MHC class I presentation assay developed by Mazzaccaro et al. in 1996, was used to select mutant strains of rBCG expressing the SIINFEKL epitope that generate improved SIINFEKL presentation by rBCG infected macrophages [165]. In the second tier of the screen, tetramer assays were used to monitor SIINFEKL-specific CD8+ T cell responses elicited in H-2K^{b} mice by the selected rBCG mutant organisms. Thirty seven strains were identified through this work that elicited higher primary tetramer responses in mice. A smaller subset of these strains, 17 strains, also primed for better tetramer responses generated following boosting with a heterologous vector construct.

Increased bacterial burdens and pathogenicity could lead to stronger immune responses. While this would be undesirable for a vaccine, it would also be consistent with the increased immunogenicity observed in this study. In order to rule this out as an explanation for the increased immunogenicity of the selected strains of rBCG, we assessed the in vivo growth kinetics of selected rBCG mutant strains and confirmed that their increased immunogenicity was not a consequence of increased in vivo growth of the organism. In fact, better control of the mutant than the parental strains was observed. This finding suggests that the main mechanism of immunogenicity is not increased in vivo growth of the strains. Rather, it is likely a molecular mechanism as a consequence of the mutations present in these strains. In fact, since the mice inoculated with the mutant strains had lower bacterial burdens after inoculation, vaccines constructed with these strains may actually have better safety profiles than the parental BCG organisms.

Transposon mutant strains K14 and AZ11 were 2 of the 37 strains identified that generated both increased in vitro IL-2 responses and increased in vivo primary tetramer responses. The location of the transposon disruption was identified in each of these strains through sequencing of the genomic DNA from the inverted repeats flanking either side of the transposon. The disruption in K14 was in the BCG gene BCG_{—}1790. In M. tuberculosis, the gene homologous to this is Rv1751. This mycobacterial gene encodes an oxidoreductase enzyme that may play a role in nitrogen metabolism. In mutant strain AZ11 the transposon was found to lie in gene BCG_{—}3445, the first member of the two-gene operon containing BCG_{—}3445 (echA18) and BCG_{—}3446 (amiD). EchA18 is a probable enoyl CoA-hydratase that is predicted to metabolize fatty acids. The M. tuberculosis homologue of echA18 is divided into echA18 (Rv3373) and echA18′ (Rv3374). In M. bovis and M. bovis BCG, a basepair T→G transversion causes echA18 to be expressed as a single gene product.

Because the transposon disruption may have polar effects on downstream genes in the operon, the disruption of BCG_{—}3446 (amiD) may be responsible for the increased MHC class I presentation observed in response to AZ11. BCG_{—}3446 and its homologue in M. tuberculosis Rv3375 are the gene amiD, which affects peptidoglycan (PG) synthesis and turnover. The enzyme AmiD is a lipoprotein located on the extracellular wall of the bacterium that catalyzes the turnover of PG fragments during cell wall remodeling [175, 176]. Mutant E. coli strains deficient for the amiD gene release large amounts of PG peptides into the extracellular medium. Because PG triggers TLR-2 activation, which in turn limits IFN-α and IFN-β production and ultimately inhibits cross presentation, the inactivation of amiD may be responsible for the phenotype of AZ11 [130].

Although a number of reported phenotypes of mycobacteria were at first attributed to a site-directed mutation or a random disruption, it was ultimately shown that the phenotype was a consequence of a second mutation in a completely unrelated pathway. The high rate of mutation of molecules that have a critical impact on immunologically related phenomenon suggests that it would be critical to rule out secondary mutations in these selected mutant strains of rBCG. To this end, both K14 and AZ11 were complemented with unmutated sections of DNA that spanned the transposon disruption. Because the strains K14 and AZ11 contain a SIINFEKL-expressing plasmid maintained by kanamycin resistance and a mariner transposon mutation maintained by hygromycin, we chose to maintain the additional plasmid DNA introduced into these bacteria by apramycin selection.

Apramycin resistant cosmids, apramycin resistant multicopy-episomal plasmids, and apramycin resistant single copy integrative plasmids were available for performing complementing studies on the mutant strains of rBCG. Cosmids are able to carry the largest amount of foreign DNA, but cosmids built with apramycin resistant arms proved to be unstable upon insertion into rBCG clones, and recombination occurred, resulting in maintenance of the apramycin resistance gene but loss of the complementing DNA. For this reason, cosmid complementation of these strains could not be performed.

Complementation of the mutant rBCG strains could not be performed with a multicopy apramycin resistant plasmid. Both the plasmid pMV261-19 kDaSIINFEKL and the apramycin resistant multicopy plasmid have an origin of replication that is pAL5000 based. Competition between the two plasmids is therefore considerable. With only 6-8 copies of the pAL5000 replicon tolerated in each bacterium, it is quite likely that the copy number of the pMV261-19 kDaSIINFEKL plasmid or the copy number of the apramycin resistant plasmid would be significantly biased, even if the rBCG strain was grown in dual selection media (kanamycin, apramycin) [134].

The integrating apramycin resistant plasmid pYUB 1141 makes use of the L5 phage integration machinery, which targets the attB core of BCG tRNA^{GLY}. Using this single copy integrating plasmid allows us to maintain one functional copy of the gene of interest in these bacteria under the control of the endogenous promoter. Because of the new location of the gene of interest in the tRNA^{GLY} site, the gene may not be subject to the same distal effects as the gene in its usual location. Maintaining the gene of interest as one copy under its original endogenous promoter comes as close as possible to modeling wild type gene expression. The entire gene BCG_{—}1790, including its endogenous promoter, was PCR-amplified from wild type BCG Danish and cloned into a single-copy integrating plasmid to create pYUB 1141-K14. Similarly, the entire 2 gene operon, containing echA18 and amiD and their endogenous promoter, was also cloned into the integrating plasmid creating pYUB1141-AZ11.

PCR indicated that complementation of these two strains using a single copy integrative plasmid was successful at a genetic level. Strains K14 and AZ11 were selected for this study because they generated transgene product-specific CD8+ T cell responses that were greater than those generated by the parental strain of rBCG. The complemented strains of K14 and AZ11 generated CD8+ T cell responses that were equivalent to those induced by the parental SIINFEKL-expressing rBCG. This indicates that for both K14 and AZ11, the transposon disruption is responsible for the phenotype of the rBCG-induced increase in the observed CD8+ T cell responses.

The data generated in the present study provides little information as to the mechanism underlying the increased MHC class I presentation of the transgene product. The possibility that this effect could be a consequence of larger amounts of transgene product produced was ruled out by Western blot analysis of the in vitro cultures showing that all of the selected mutant and parental rBCG strains produced comparable levels of transgene protein. Alternatively, this effect could be a consequence of altered protein processing or increased access to the MHC class I loading machinery. It is also possible that loaded MHC class I molecules are maintained on the surface of the APC for longer periods of time, that there is an increased production of costimulatory molecules, or that there is an altered cytokine milieu that favors CD8+ T cell development. Finally, the parental rBCG construct may kill CD8+ T cells or APCs and this killing function may be lost as a consequence of transposon disruption [177].

CD8+ T cell induced by the 37 mutant strains identified in the screens were 2- to 3-fold greater than those induced by the unmutated parental rBCG strain. A smaller subset of 17 selected rBCG strains also generated increased transgene product-specific CD8+ T cell responses when a heterologous boosting immunogen was delivered in association with the priming rBCG immunization. Those mutant rBCG constructs that induce increased prime and heterologous boost responses are promising for use in vaccine development. Increased transgene product-specific CD8+ T cell responses could be harnessed in the creation of an rBCG vector for vaccination against pathogens whose control is mediated through a cellular immune mechanism.

### III. Novel rBCG Vaccine Vectors Prime for Increased Anti-SIV T Cell Responses Equivalent to Plasmid DNA

### Abstract

Through the use of a two-tiered screen, we have identified 17 strains of recombinant Mycobacterium bovis BCG (rBCG) that generate increased CD8+ T cell responses. Here, we have identified the 17 rBCG genes implicated in modulating the host's CD8+ T cell response. The predicted functions of the disrupted genes include secreted pathogenic proteins, protein-modification enzymes, transcription factors, enzymes involved in cellular function and metabolism, and genes with no known function. Using a site-directed method of gene deletion, we have created novel rBCG strains with disruptions in two of these genes and demonstrated that the new strains and the respective transposon mutant BCG strains increase immunogenicity to a comparable degree. Then, we introduced the SIV gag gene into these strains and demonstrated the ability to elicit robust Gag-specific CD8+ T cell responses upon heterologous boost in a mouse model, responses that are several fold greater than the responses induced using the unmutated strain of rBCG as a priming immunogen. Importantly, these new second generation strains of rBCG have the ability to generate memory CD8+ T cell responses that are comparable to those generated by plasmid DNA vaccines.

### Materials and Methods

Identification of transposon location. Nine ml of each transposon mutant was grown to an optical density (OD) of 0.8. One ml of 10% glycine (wt/vol) was added to 9 ml BCG culture for 24 hrs. Cultures were then pelleted, incubated with Lysis Buffer for 1 hr at 65° C. and genomic DNA was isolated using the DNeasy Qiagen kit. Genomic DNA was digested for 2 hrs with BssHII, and circularized by ligation with T4 DNA ligase for 1 hr to make plasmids bearing the pir-dependent origin of replication. These plasmids were subsequently transformed into pir2000 electrocompetent cells. Cells were plated on LB agar plates with hygromycin at a concentration of 100 μg/ml. Pir2000 cells containing viable plasmids grew into colonies, and plasmid DNA was isolated using the Qiagen Miniprep Spin Kit. Plasmids were sequenced using the primers “LIR” and “RIR” that bind internally to the LIR and RIR of the transposon. Sequences were aligned with the BCG Pasteur sequence AM408590 using NCBI Blast to identify the genes surrounding the site of transposon disruption, allowing the identification of the disrupted pair of nucleotides.

Creation of DNA vaccine. A plasmid DNA vaccine expressing the 19 kDaSIINFEKL sequence was created by PCR using the template plasmid pMV261-19 kDaSIINFEKL. PCR product was restriction enzyme digested and ligated into the multiple cloning site on the plasmid pVRC2000 (kindly provided by Dr. Gary Nabel, NIH). Ligation product was transformed into DH5a cells (NEB). The plasmid transgene region was sequenced prior to large scale preparation, and sufficient quantities for murine immunization studies were obtained using a Qiagen Maxiprep kit.

Cloning of allelic exchange substrates. Allelic exchange substrates pAES2589-Operon and pAES0546c-Gene were created. For pAES2589-Operon, a 561 bp homologous fragment flanking the left (2587-L) side of the BCG_{—}2587-2590 operon and a 676 bp homologous fragment flanking the right hand (2590-R) side of the operon were amplified by PCR. For pAES0546c-Gene, an 822 bp homologous fragment flanking the left side of BCG_{—}0546c (0546c-L) and a 541 bp homologous fragment flanking the right hand side (0546c-R) of the gene were amplified by PCR.

All reactions were performed using the BD Advantage Taq polymerase, with a template of BCG Danish genomic DNA. Denaturation occurred at 95° C., and annealing and extension were performed at 65° C. The 2587-L strand was digested with the restriction enzyme Van91I and the 2590-R strand was digested with the restriction enzyme BstAPI. The 0546c-L fragment was digested with Van91I, and 0546c-R was digested with BstAPI. These fragments were cloned into the plasmid p0004S. In brief, 2 μg of the plasmid p0004S was digested with Van91I. The digestion mixture was run on a 1% gel for 1 hr and the bands containing the hygromycin resistance gene (H, 3672 bp) and the E. coli origin of replication (0, 1614 bp) were isolated from the gel. A four-part ligation was performed using the L, H, R, and O fragments creating a circular plasmid.

Ligation mixtures were transformed into DH5a cells (NEB) and plated on LB agar plates containing 100 μg/ml hygromycin. Multiple colonies were selected and grown in LB media with 100 μg/ml hygromycin. Plasmid was isolated using the Qiagen Miniprep kit and sequenced using the primers “HL”, “HR”, “OL”, and “OR”. Plasmids without point mutations were used for the creation of the phasmids phAE2589-Operon and phAE0546c-Gene.

Phasmid creation. pAES2589-Operon and pAES0546c were digested with the enzyme PacI. Ten μg of DNA encoding the TM4 phage, phAE159, was also digested with PacI, heat inactivated at 65° C., dephosphorylated with SAP for 20 minutes, and then purified using the Qiagen PCR clean up kit. Purified phAE159 DNA digested with PacI was ligated to pAES2589-Operon and pAES0546c-Gene DNA using T4 DNA ligase. The resulting mixture was packaged into lambda phage heads using the MaxPlax™ Lambda Packaging Extract. This reaction was stopped after 2 hours using buffer MP and the mixture was used to transduce HB101 cells. These cells were plated on LB agar plates containing 100 μg/ml hygromycin. Phasmid DNA was isolated from four selected colonies using a Qiagen miniprep kit. Ten μl of phasmid DNA was digested using PacI to confirm the successful introduction of the new AES. Successful incorporation was observed in 3 of the 4 colonies.

Phage amplification. Four μl of prophage DNA was used to transform M. smegmatis mc2155. Plaques resulting from the transformation of phasmid DNA into M. smegmatis at 30° C. were chosen for further transduction and amplification in the M. smegmatis host. High titer phage, 10¹⁰ PFU, was obtained after 3 successive rounds of amplification.

BCG transduction. High titer phage from phAE0546c was used to transduce BCG Danish. 10⁹ CFU wild type BCG Danish at an optical density of 1 were pelleted and resuspended in 1 ml buffer MP with 10¹⁰ PFU of the phage phAE0546c-Gene. Cells were incubated overnight at 37° C. and plated onto 7H10-ADS plates containing 100 μg/ml hygromycin. Plates were incubated for 3 weeks at 37° C.

Transformation. A 15 ml culture of BCG was grown to an OD=1, pelleted at 3000 rpm, and washed three times with a solution of 10% glycerol. The pellet was resuspended in 200 μl of 10% glycerol, mixed with 200 ng DNA of the plasmid pMV261-19 kDaSITINFEKL, pSL10, or pSL7, and incubated for 20 minutes. Cells were transformed by electroporation (2.5 kV, 25 mF, 1000 ohms). Electroporated cells were incubated in 7H9 media overnight and then plated on 7H10 plates with 20 μg/ml kanamycin (pMV261-19 kDaSIINFEKL) or 30 μg/ml apramycin (pSL10, pSL7). Three weeks later, colonies were selected and grown to an optical density of 1.

Colony PCR. Forty nine p1 of a stock PCR mixture containing the BD Advantage Taq polymerase, dNTP, buffer and primers to amplify the gene of interest were aliquoted into PCR tubes. A small but visible amount of bacterial colony was taken off the plate (estimated volume 1 μl) and added to the appropriate tube. For culture PCR, 1 μl of BCG culture at an OD>0.5 was added to the PCR reaction. Samples were denatured at 95° C. for 10 minutes prior to PCR. Fifteen p1 of a 501 PCR reaction was run in each lane.

Southern blot. Genomic DNA was isolated from 5×10⁹ CFU of actively growing log phase (10 ml, OD=1)ᵣBCG cultures. Cultures were grown to an OD=0.8, at which point glycine was added to the culture to a final wt/vol of 1%, and cultures were grown for 24 more hours. Samples were pelleted, resuspended in lysis buffer containing lysozyme, and heated to 65° C. for 30 min. The Qiagen DNEasy kit was used to isolate genomic DNA from the samples. Genomic DNA was digested with PstI and NotI at 37° C. for 2 hours and loaded into a 1% agarose gel. Prior to transfer, the gel was soaked in an ethidium bromide solution and visualized under UV light to confirm equal DNA loading. Transfer by capillary action was done overnight. Blotting was done using a DIG labeled 300-400 basepair probe, and visualized using the DIG DNA labeling kit (Roche Applied Science).

Western blot. For Western blots assessing SIINFEKL expression, 2.5×10⁶ CFU (10 μl of a culture at an optical density of 0.5) was isolated from actively growing log phase BCG cultures. For Western blots assessing SIV Gag expression, 2.5×10⁷ CFU (100 μl of a culture at an optical density of 0.5) was isolated. Bacilli were pelleted and washed once with 100 μl of extraction buffer containing a protease inhibitor. The pellet was then resuspended in extraction buffer containing the protease inhibitor, LDS, and a reducing agent (Nupage, Invitrogen), and heated to 95° C. for 10 minutes. Samples were loaded into a 10% bis tris 15 lane gel (Invitrogen) and run for 90 minutes at 100 volts. Protein was transferred to a PVDF membrane at 30 volts for 1 hour, and stained with antibody for 1 hour. For blots assessing SIINFEKL expression, a primary rabbit polyclonal serum was used as a primary antibody and a secondary rat anti-rabbit antibody conjugated antibody was used for detection. For blots assessing SIV Gag expression, a high affinity rat monoclonal antibody directed against the HA tag (clone 3F10) conjugated to HRP was used for detection. Visualization was done using the Roche Chemiluminescence Kit.

p27 ELISA. Supernatants from BCG cultures at an OD of 1 were collected for assessment of secreted SIV Gag using the commercially available p27 Antigen ELISA kit from ZeptoMetrix. Two hundred μl of supernatant was incubated in each well at 37° C. for 2 hours. Wells were aspirated, washed, and then incubated with an SIV-p27 Detector Antibody for 1 hour at 37° C. A Streptavidin Peroxidase Working Solution was added to each well and colorimetric analysis was performed using a SpectraMax Plus plate reader.

Immunization protocol. One hundred million CFU from an actively growing log phase BCG culture (between OD=0.6 and 0.9) was isolated, pelleted and resuspended in 1 ml PBS-tween 20 0.2%. One hundred μl (10⁷ CFU) was injected IV into each of 4-8 C57Bl/6 mice per group. Twenty five μg of plasmid DNA was injected into each hind leg quadriceps muscle for a total of 50 μg DNA/mouse. One million PFU rAd5-SIINFEKL or 10⁷ PFU rAd5-SIV Gag was injected IM into the hind leg quadriceps of mice.

Tetramer staining. Seven to 14 days after vaccination, 100 μl of peripheral blood was collected from each mouse into RPMI via cheek bleeds, ACK treated to remove red blood cells, and stained using an H-2K^{h}-SIINFEKL-PE or H-2D^{b}-AL11-PE tetramer and anti-CD8-PerCP-Cy5.5 antibodies. Results are displayed as the CD8+ T cells that stained positive by tetramer as a percent of the total number of peripheral blood CD8+ T cells.

### Results

Identification of BCG Genes that are Associated with Increased MHC Class I-Restricted CD8+ T Cell Responses

We developed a two-tiered screen to identify rBCG transposon mutant organisms that generate both increased MHC class I presentation in vitro and increased CD8+ T cell responses in vivo. For this screen we created a library of rBCG transposon mutant strains expressing a model transgene coding for the dominant CD8+ T cell epitope SIINFEKL. We identified 122 transposon mutants that generated a higher level of MHC class I presentation of SIINFEKL in vitro by infected macrophages than the level generated by the unmutated parental strain of rBCG expressing SIINFEKL. The increased macrophage MHC class I presentation of SIINFEKL was associated with the induction of increased SIINFEKL-specific CD8+ T cell responses in H-2K^{b} mice following in vivo inoculation with 17 of these 122 mutant strains of rBCG.

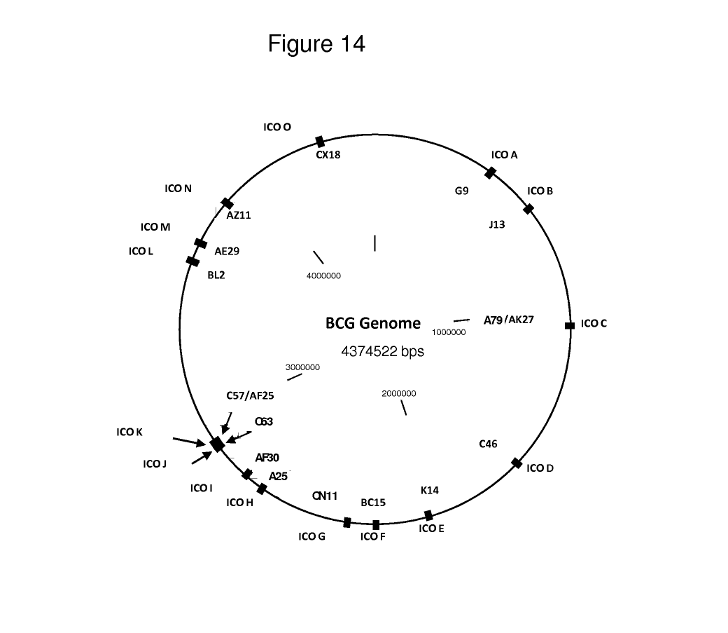

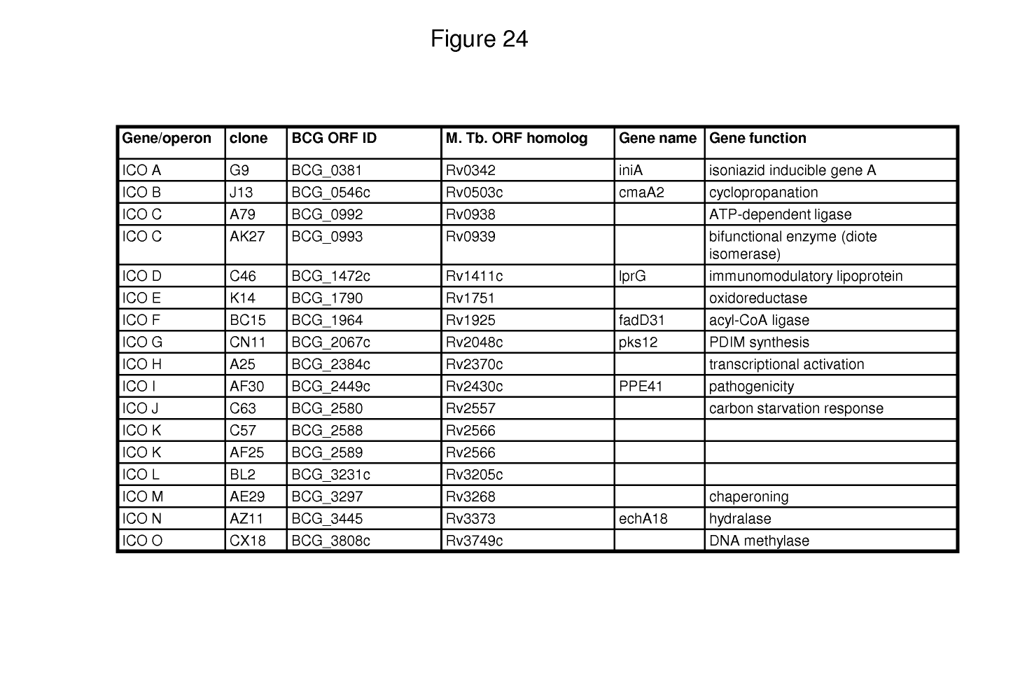

We isolated genomic DNA from these 17 selected strains of rBCG that generated increased primary and secondary transgene product-specific CD8⁺ T cell responses in vivo and performed sequencing to determine the location of the transposon insertion. These 17 genes mapped to 15 unique genes/operons, and their locations within the BCG genome are shown in FIG. 14. Surprisingly, the gene locations are distributed throughout the BCG genome, and no pathogenicity island could be implicated in BCG-specific CD8+ T cell responses. Two of the immunomodulatory operons identified in the screening process were each identified by two independent transposon insertions (ICO K:C57 and AF25; and ICO C: A79 and AK27). We named the 15 unique strains Inhibitor of Class One (ICO) strains A-O. As shown in the table of FIG. 24, the ICO strains (transposon mutant strains) are listed in the first column, the specific library clone numbers are listed in the second column, the BCG open reading frames (ORFs) disrupted in the strains are listed in the third column, and the corresponding homologous genes in M. tuberculosis H37Rv are listed in the fourth column. For genes encoding ORFs that have been identified and characterized, the name and function of the gene are listed and these perform cellular functions ranging from pathogenicity to DNA repair (ICOs A, B, D, F, G, I, and N). For ORFs that have not been previously characterized, the name of the gene has been left blank, and the putative function based on conserved motifs is listed. These include transcriptional regulation, DNA modification, protein modification, metabolic regulation and protein transport. Consistent with the variety of functions associated with the affected genes, the gene locations are distributed throughout the BCG genome. No “islands of pathogenicity” could be identified in the suppression of CD8⁺ T cell responses. In two instances two transposons were identified in the same operon (A79 and AK27, AF25 and C57).

Comparison of the Immunogenicity of Selected rBCG Mutant Strains with an Existing Modified BCG Microorganism and Plasmid DNA

We tested various strains of BCG of the present invention to determine their usefulness as priming vectors in a heterologous prime/boost vaccination regimen. To determine the effectiveness of these novel strains of BCG as priming vectors, we compared their immunogenicity to that of a prototype priming immunogen, plasmid DNA, as well as a rBCG vaccine strain currently under evaluation in human trials, AERAS 401. AERAS 401 is a rBCG strain modified to express the perfringolysin gene from Clostridium perfringens, allowing the bacteria to form pores in the endosomal compartments and enhancing antigen access to the MHC class I pathway of infected cells. To compare the immunogenicity of our newly generated strains of BCG to the AERAS 401 BCG strain, we transformed AERAS 401 with the plasmid pMV261-19 kDaSIINFEKL and confirmed expression of the transgene in transformed clones by PCR and Western blot analysis.

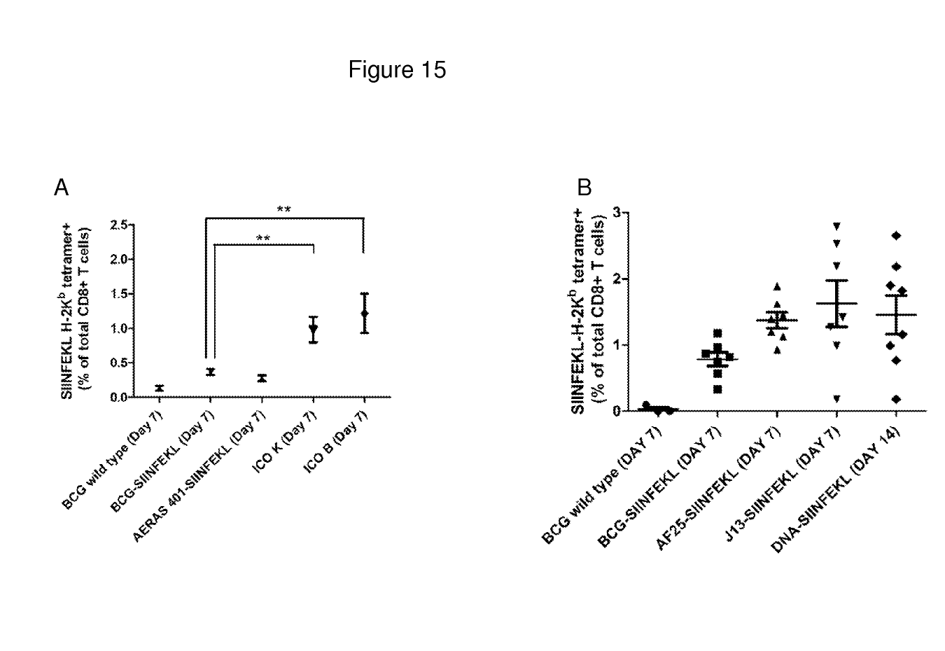

Transposon mutant strains AF25 (ICO K) and J13 (ICO B) expressing SIINFEKL were compared to AERAS 401 expressing SIINFEKL (FIG. 15A) and to plasmid DNA for their ability to stimulate a primary SIINFEKL-specific CD8+ T cell response in vivo (FIG. 15B).

Time courses of tetramer responses were monitored to determine the kinetics of the responses generated by rBCG, AERAS 401, and a plasmid DNA vaccine. The peak tetramer response to the rBCG constructs was on day 7 post-vaccination, whereas the peak tetramer response to the plasmid DNA vaccine occurred on day 14 post-vaccination. We then compared the magnitude of the peak tetramer responses generated by each vector (FIG. 15B). Wild type BCG vectoring SIINFEKL stimulated a peak SIINFEKL-specific CD8+ T cell response with a mean of 0.78%; transposon mutants AF25 and J13 stimulated peak SIINFEKL-specific CD8+ T cell responses (means of 1.37% and 1.63%, respectively) that were comparable in magnitude to the mean peak response stimulated by the plasmid DNA vaccine (1.36%).

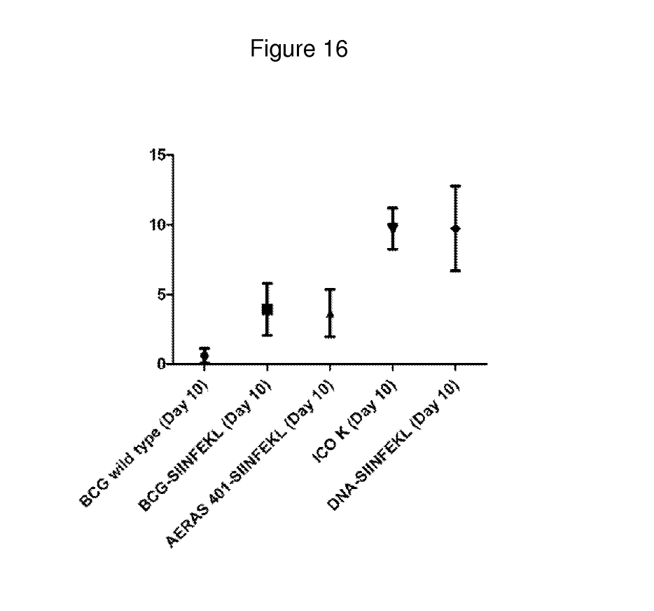

To determine whether the increased primary responses induced by the novel strains of rBCG primed for increased secondary responses following boosting with a heterologous vector, all groups of primed mice were boosted with a suboptimal dose (1×10⁶ vp) of rAd5-SIINFEKL (FIG. 16) 10 weeks after the priming vaccination. Following boosting, mice primed with mutant rBCG strain ICO K generated mean secondary SIINFEKL-specific CD8 T cell responses (9.7%) that were significantly greater than the responses by groups of mice primed with either the parental- or the AERAS 401-SIINFEKL strains (3.9% and 3.4%, respectively). The magnitudes of the secondary responses in mice primed with mutant rBCG strains were comparable to the responses primed by plasmid DNA vaccination (9.7%). Moreover, at 10 days after boosting with rAd5-SIINFEKL, mice that were primed with rBCG without the SIINFEKL transgene did not generate a significant SIINFEKL-specific CD8⁺ T cell response. Therefore, the responses observed in the SIINFEKL-primed groups of mice represented secondary CD8⁺ T cell responses rather than de novo primary responses to the rAd vector. rAd5-SIINFEKL-boosting of mice primed with wild type BCG Danish that did not express the SIINFEKL epitope elicited a peak tetramer response with a mean of 0.6%.

Reconstruction of Transposon Mutant Strains by Site-Directed Mutagenesis.

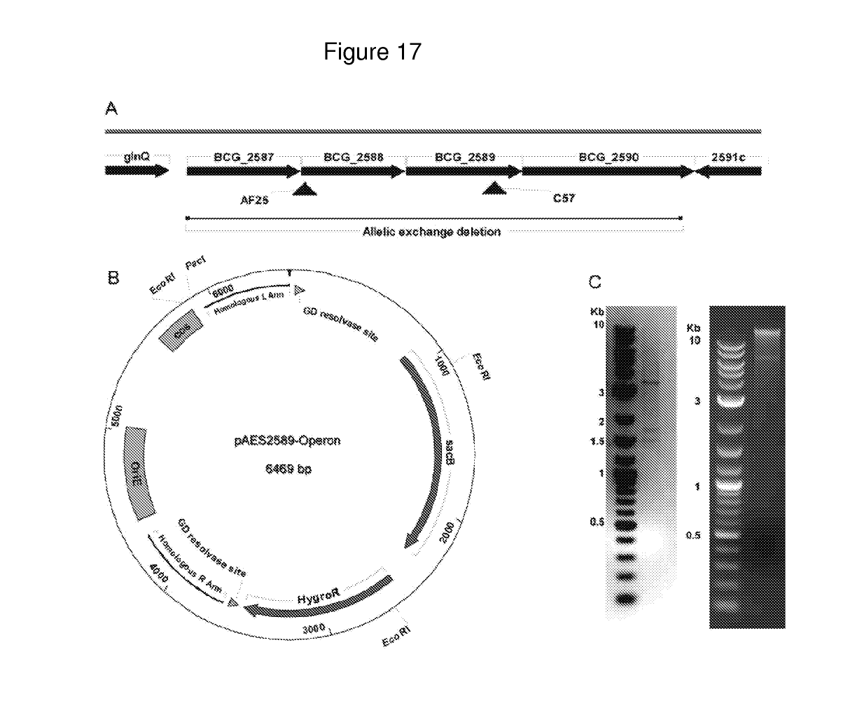

Upon repeated testing, rBCG strains with transposon insertions in the ICO K operon (clones C57 and AF25) and the ICO B gene (clone J13) consistently primed for primary and secondary transgene product-specific CD8⁺ T cell responses that were significantly greater than those elicited by the parental strain of rBCG. To confirm that the increased immunogenicity of these rBCG strains was a consequence of the specific transposon insertions rather than unrelated mutations at other gene loci, we reconstructed these mutant rBCG strains from wild type BCG Danish by site-directed mutagenesis using a well established technical strategy (Bardarov et al., 2002). Allelic exchange substrates (AES) targeting the ICO K operon and the ICO B gene were synthesized that would be capable of replacing the targeted genes with an antibiotic resistance gene. To create a vaccine strain of BCG with the same phenotype as the ICO K BCG strains, we made an AES that targeted the entire BCG_{—}2587-2590 operon (FIGS. 17A and 17B). This allelic exchange substrate was constructed from 4 separate DNA fragments: the Origin (O) fragment contains an origin of E. coli replication for amplification; the Left arm (L) and Right arm (R) fragments are homologous to the BCG genomic DNA flanking the gene of interest; and these are cloned on either side of a fragment containing hygromycin-selection/sacB-counterselection markers (H fragment). Digestion of the pAES2589-Operon plasmid by EcoRI yields three distinct bands on a 1% agarose gel: the 1516 bp fragment contains the sacB gene, the 1710 bp fragment contains the L arm, and the 3243 bp fragment contains the origin of E. coli replication and the R arm (FIG. 17C, left panel). This allelic exchange substrate was cloned into the 47 kb prophage phAE159 to create the phasmid phAE2589-Operon. Digestion of phAE2589-Operon with PacI yields a 6.5 kb AES and a 47 kb prophage backbone encoding the temperature-sensitive TM4 phage (FIG. 17C, right panel).

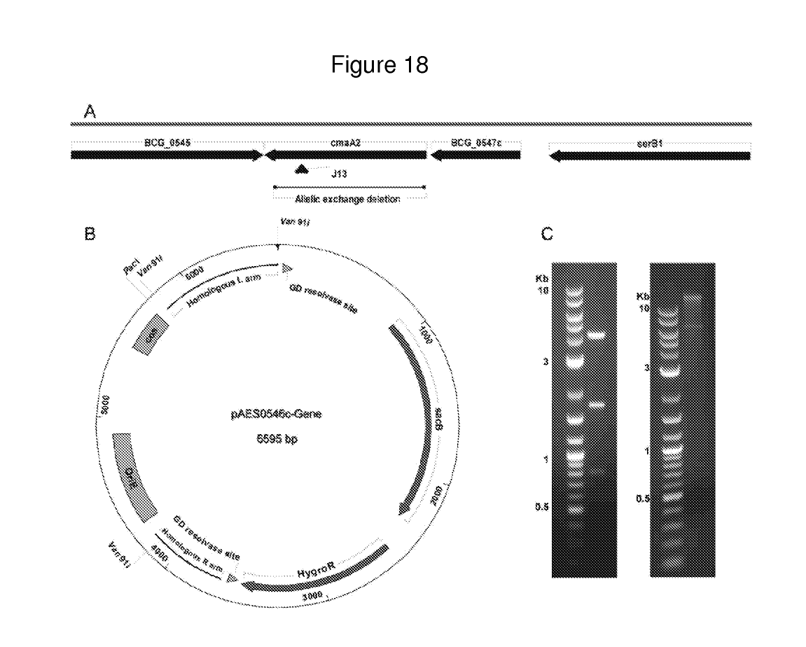

To reconstruct the ICO B strain, we generated an AES to specific for the cmaA2 gene (BCG_{—}0546c) (FIG. 18A). Homologous arms flanking the J13 gene cmaA2 were ligated with H and O arms to create the AES pAES0546c-Gene (FIG. 18B). Digestion of this plasmid by Van91I yields a 795 bp fragment with the L arm, a 1654 bp fragment with the O arm, and a 4146 bp fragment containing the L arm and the H arm (FIG. 18C, left panel). This AES was subsequently cloned into the prophage phAE159, and digestion of the resulting phasmid phAE0546c with PacI yields a band of 6.6 kb corresponding to the AES and a band of 47 kb corresponding to the prophage backbone (FIG. 18C, right panel).

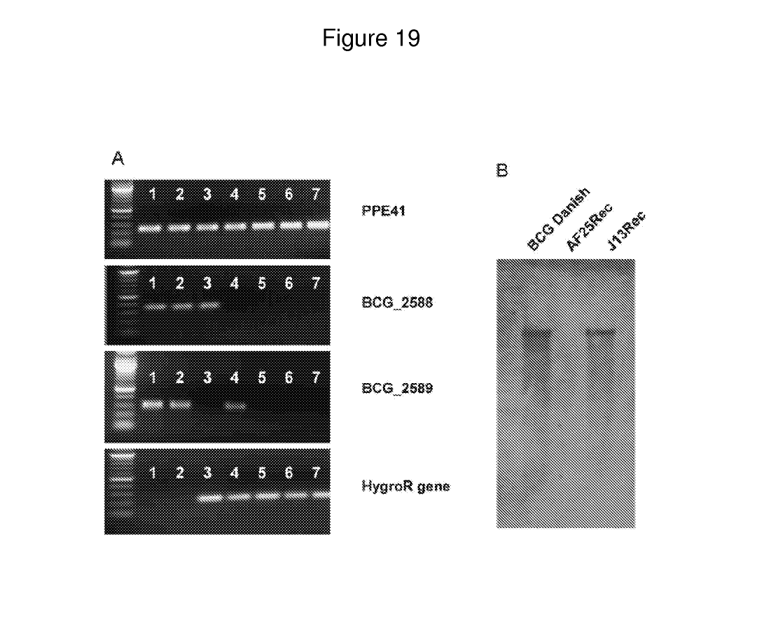

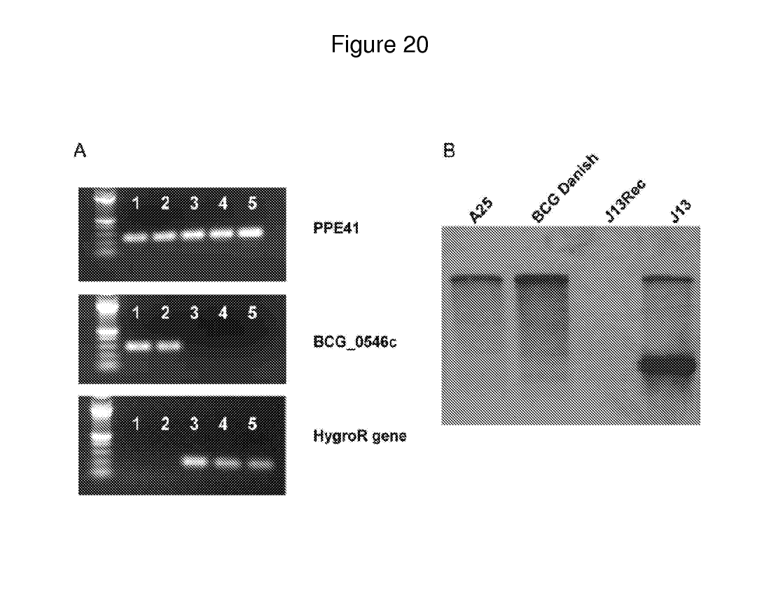

These AES were subsequently cloned into the prophage phAE159 and used to transduce wild type BCG. The resulting reconstructed rBCG strains lacking BCG_{—}2587-2590 and BCG_{—}0546c were termed ICO K Rec and ICO B Rec, respectively. Successful construction was confirmed by PCT and by Southern blot (FIGS. 19B and 20B).

Gene Deletion Using Specialized Transduction

Phages were created from the phasmids phAE2589-Operon and phAE0546c-Gene using M. smegmatis as an intermediate host. High titer phages were used to transduce BCG at the nonpermissive temperature of 37° C. Incubation at the nonpermissive temperature prevents the TM4 phage from undergoing replication. The DNA circularizes and catalyzes crossing over with the host genomic DNA within homologous regions, resulting in a replacement of the gene of interest with the hygroR/sacB cassette. Gamma delta resolvase sites flank the hygroR/sacB cassette for unmarking transduced BCG. The resulting reconstructed strains were called AF25Rec (also ICO K Rec) and J13Rec (also ICO B Rec).

### Confirming Gene Deletion

We used PCR to screen for successful gene deletion in each of the 6 colonies resulting from transduction of BCG with phAE2589-Operon, and each of the 2 colonies that resulted from the transduction with phAE0546c-Gene. In all 6 colonies resulting from phAE2589-Operon transduction, identical PCR results were observed. Similarly, in both colonies resulting from phAE0546c-Gene transduction, identical PCR results were observed, indicating successful deletion of the targeted genes. This method of PCR gene detection identified illegitimate recombination in previous transductions at a rate of 2/10 clones; there was no indication of illegitimate recombination in any of these colonies. One clone of each transduction was transformed with the plasmid pMV261-19 kDaSIINFEKL. Two colonies from the transformation were assessed for deletions by PCR.

PCR primers were generated for PPE41 to confirm that the colonies were slow growing mycobacteria. All cultures examined were positive for the gene PPE41 (FIG. 19A). PCR primers were generated to amplify a 371 bp region in BCG_{—}2588, which was disrupted by a transposon in the AF25 transposon mutant. PCR for BCG_{—}2588 from the wild type BCG Danish culture (FIG. 19A, lane 1) produced a 371 bp fragment, confirming the presence of an intact BCG_{—}2588 gene in this bacterium. Similarly, PCR using BCG Danish containing the plasmid pMV261-19 kDaSIINFEKL as a template produced a 371 bp fragment (FIG. 19A, lane 2).

PCR for BCG_{—}2588 using the C57 transposon mutant strain of rBCG (FIG. 19A, lane 3) as a template produced a 371 bp fragment, consistent with the transposon in AF25 being located in the gene downstream of the disrupted locus in C57 (BCG_{—}2589) and not affecting the results of PCR of an adjacent gene. PCR using the transposon mutant AF25 as a template (FIG. 19A, lane 4) did not produce a 371 bp fragment. The primers for BCG_{—}2588 amplification flanked the location of the predicted transposon based on sequencing data, and with the introduction of approximately 2200 bp of foreign transposon DNA inserted between the primers, δ 1 minute extension time is insufficient time to yield a product. PCR reactions from cultures of AF25Rec, which contains a deletion in the BCG_{—}2587-2590 operon due to specialized transduction do not produce a product of 371 bp. These cultures do not have the template region to which the primers might bind due to a deletion induced by allelic exchange (FIG. 19A, lanes 5 and 6).

PCR primers were generated to amplify a 306 bp fragment within the BCG_{—}2589 gene, which contains a transposon in the C57 mutant strain of BCG. Wild type BCG (FIG. 19A, lane 1), BCG transformed with the pMV261-19 kdaSIINFEKL plasmid (FIG. 19A, lane 2) and the transposon mutant AF25 (FIG. 19A, lane 4) all contain an undisrupted BCG_{—}2589 gene, and PCR reactions generated from colonies from these cultures therefore produce a 306 bp fragment. C57 (FIG. 19A, lane 4) contains a transposon in the region flanked by these PCR primers, and therefore no product is created using these PCR primers. Similarly, a PCR product is not formed in the reaction using AF25Rec as a template because there is a complete deletion of the operon in this strain.

PCR primers were also generated to amplify a 221 bp fragment of the hygromycin resistance gene, which was introduced into BCG by both transposon insertion and allelic exchange technology. FIG. 19A (bottom panel) indicates that there is no hygromycin resistance gene in wild type BCG or wild type BCG transformed with pMV261-19 kDaSIINFEKL. The hygromycin resistance gene was present in all transposon mutants and strains of AF25Rec that were tested.

While PCR is a highly sensitive technique to analyze the genetics of an organism, its use has limitations. The amplification of a gene of interest that is present in a small subpopulation of bacteria may give a positive signal that does not reflect the entire population of bacteria. For this reason, we used Southern blotting to confirm the genetic status of these organisms. We isolated equal amounts of genomic DNA from cultures of different BCG strains and probed the strains with a small (306 bp) fragment of DNA homologous to the BCG_{—}2589 gene (FIG. 19B). The BCG_{—}2589 gene was present in BCG Danish and the strain J13Rec, but not present in the strain AF25Rec.

BCG Danish was transduced with the phage created by phAE0546c-Gene. Cells were incubated at the nonpermissive temperature of 37° C., two colonies were chosen based on PCR confirmation of a successful gene deletion, and these colonies were transformed with the plasmid pMV261-SIINFEKL. PCR was then used to confirm the successful deletion of the gene BCG_{—}0546c in the new vaccine vector constructs.

PCR using primers for PPE41 indicated that all colonies were slow growing mycobacteria (FIG. 20A, top panel). PCR primers amplifying a 295 bp fragment of the gene BCG_{—}0546c were created and used to generate a product in BCG Danish (lane 1) and BCG pMV261-SIINFEKL (FIG. 20A, lane 2). No product was generated in the J13 transposon mutant strain, which contains approximately 2200 bp of inserted transposon DNA disrupting BCG_{—}0546c (FIG. 20A, lane 3). No product was also seen in either clonal population of J13Rec (FIG. 20A, lane 4 and 5), because the target location was successfully deleted by specialized transduction. These data demonstrate successful introduction of the hygroR/sacB cassette by specialized transduction in very specific locations, ruling out the possibility of an introduction of the cassette in a non-specific manner as seen in illegitimate recombination.

We isolated equal amounts of genomic DNA from cultures of 4 different BCG strains and probed the strains with a small (295 bp) fragment of DNA homologous to the BCG_{—}0546c gene (FIG. 20B).

Probing transposon mutant A25 yielded a band corresponding to DNA homologous to the BCG_{—}0546c probe (FIG. 20B, lane 1). This band was also observed in BCG Danish (FIG. 20B, lane 2). Therefore, neither the presence of the pMV261-19 kDaSIINFEKL plasmid nor the presence of transposon DNA in a trans location interfered with blotting of the BCG_{—}0546c gene. No hybridization to genomic DNA was observed from the J13Rec strain created by specialized transduction (FIG. 20B, lane 3). Finally, hybridization at two sites was observed for the J13 transposon mutant, consistent with a transposon disrupting the BCG_{—}0546c gene. No genetic material was deleted in this strain, but foreign transposon DNA was introduced in the middle of the gene. The probe hybridized at two different locations because the target gene was bisected by the foreign DNA with some of the probe hybridizing to DNA upstream of the transposon site, and some hybridizing to DNA downstream of the transposon.

Novel Constructs Elicit Greater Magnitude CD8+ T Cell Responses

While the transposon mutant strains AF25 and J13 induced greater magnitude MHC class I-restricted CD8+ T cell responses than those induced by wild type BCG, it was possible that the enhanced immunogenicity of these vaccine constructs was due to mutations other than those mapped by the location of the transposons. We therefore sought to prove formally that deletions of the defined single genes were responsible for the enhanced immunogenicity of AF25 and J13.

Strains AF25Rec and J13Rec were transformed with the plasmid pMV261-19 kDaSIINFEKL. Transgene expression from the new constructs AF25Rec-19 kDaSIINFEKL and from the construct J13Rec-19 kDaSIINFEKL was comparable to the transposon mutant strains AF25, C57, and J13, and was also comparable to the parental strain of BCG Danish containing pMV261-19 kDaSIINFEKL.

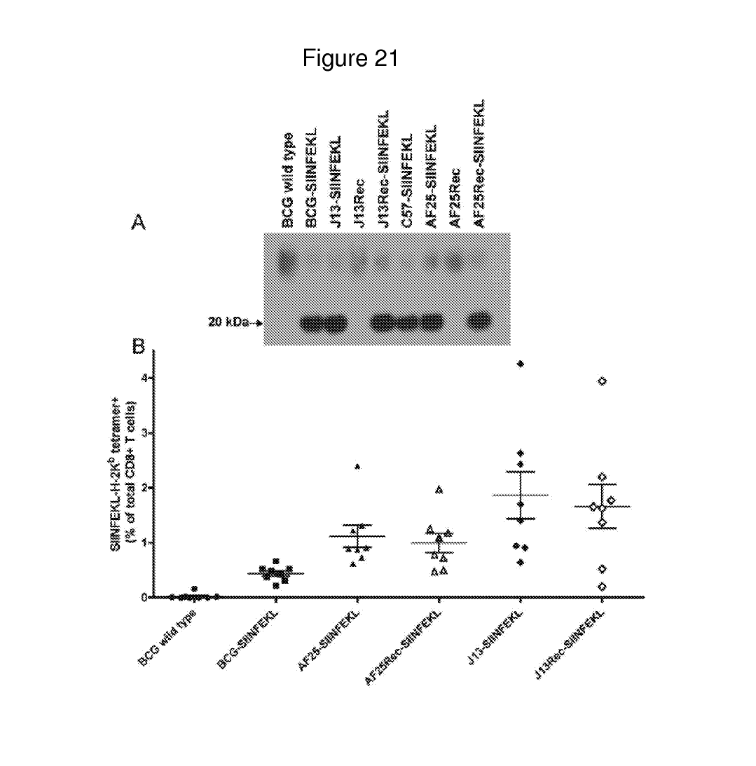

Because these constructs now expressed the transgene epitope SIINFEKL, which, when presented by APC in the context of the MHC class I molecule H-2K^{b}, stimulates an immunodominant CD8+ T cell response, we were now in a position to evaluate the relative immunogenicity of these constructs (FIG. 21). Wild type BCG Danish expressing no transgene elicited a background mean tetramer response of 0.02%. Wild type BCG Danish expressing the 19 kDaSIINFEKL protein elicited a mean peak SIINFEKL-specific response of 0.44% of the CD8+ T cells in peripheral blood. The transposon mutant AF25 elicited a mean response of 1.11%, greater than twice the magnitude induced by the parental construct, and transposon mutant J13 elicited a mean response of 1.86%, greater than 4 times the magnitude induced by the parental construct. Importantly, the constructs J13Rec and AF25Rec were comparable to the transposon mutants in their immunogenicity. Therefore, the single gene deletions identified in the transposon mutants were responsible for the enhanced immunogenicity of the rBCG strains.

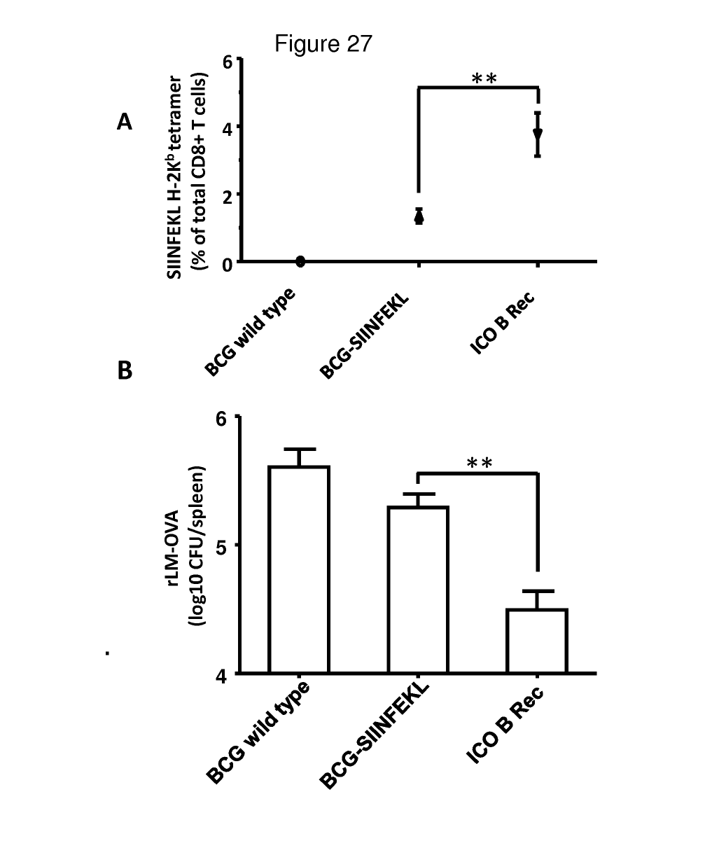

The CD8⁺ T cell responses generated by these vectors were then assessed for their ability to control a pathogenic challenge with Listeria monocytogenes. Mice primed with the vector strain ICO B Rec expressing SIINFEKL demonstrated a significantly augmented CD8⁺ T cell response against SIINFEKL than mice primed with BCG-SIINFEKL (FIG. 27A). These mice were challenged with 5×10⁴ CFU of a strain of recombinant Listeria monocytogenes expressing ovalbumin (rLM-OVA), and Lysteria burden in the spleen was assessed after 3 days as a measure of protection (FIG. 27B). Listeria burden at this time point was more than six-fold lower in the mice that had been vaccinated with ICO B Rec than in mice vaccinated with BCG-SIINFEKL. Therefore, the CD8⁺ T cell responses generated by the novel mutant ICO B Rec were functional and fully capable of controlling a pathogenic microbial challenge.

Immunogenicity of Novel rBCG Vectors when Used in a Prime/Boost Regimen

We finally assessed the immunogenicity of these novel rBCG vectors expressing a different transgene in a prime/boost strategy with a recombinant Ad5 immunogen. We chose to use SIV gag as the transgene for these experiments because this transgene contains the murine H-2D^{b}-restricted immunodominant AL11 epitope and the rhesus monkey Mamu-A*01-restricted immunodominant epitope p11C. The SIV gag gene was cloned into a multicopy episomal plasmid, and two E. coli-mycobacterial shuttle plasmids were created. One plasmid, pSL10, expressed a fusion protein containing the signal sequence from the Ag85a secreted mycobacterial protein, the full length SIV Gag protein, and an HA tag at the C terminus. The other plasmid, pSL7, expressed a fusion protein containing the N terminus acylation sequence of the 19 kDa (Rv3763) protein, the full length SIV Gag protein, and an HA tag at the C terminus. The predicted protein size without modification was 64 kDa.

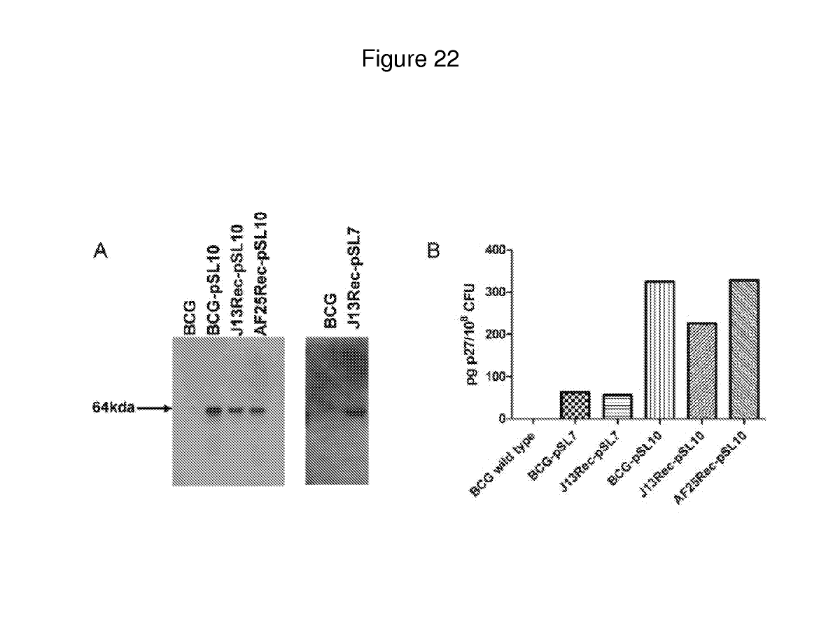

pSL10 was transformed into wild type BCG Danish, AF25Rec created by specialized transduction and J13Rec created by specialized transduction. Expression of the 64 kDa fusion protein was assessed by Western blot using a high affinity anti-HA antibody. A single band at 64 kDa was observed in rBCG strains transformed with the pSL10 plasmid (FIG. 22A, left panel). Samples were normalized so that identical quantities of CFU of each sample were processed and loaded in each lane. Expression of the Ag85-Gag-HA fusion protein was comparable between all strains containing the pSL10 plasmid. pSL7 was transformed into wild type BCG Danish and the J13Rec strain of BCG. Expression of the 64 kDa fusion protein 19 kDa-Gag-HA is demonstrated in the J13Rec strain in the right panel of FIG. 22A.

Secretion of the SIV Gag-containing constructs was assessed by p27 ELISA (FIG. 22B). Equivalent levels of 19 kDa-Gag-HA secretion were observed from wild type BCG and J13Rec constructs containing pSL7. All constructs containing the pSL10 plasmid secreted comparable levels of SIV Gag into the supernatant, levels that were greater than the levels secreted by constructs containing the pSL7 plasmid. Levels of Ag85-Gag-HA ranged from 200 to 330 pg of p27 per 10⁸ CFU, while levels of 19 kDa-Gag-HA ranged from 50-70 pg of p27 per 10⁸ CFU.

These strains of BCG were injected into C57Bl/6 mice to assess the Gag-specific CD8+ T cell responses that they elicited. Primary AL11 tetramer responses to all constructs were less than 0.2%. A suboptimal boost of 10⁷ PFU rAdS-SIV Gag was therefore given at 8 weeks, and Gag-specific CD8+ T cells were assessed 10 days later. This suboptimal boosting dose was chosen after titration studies to provide the best discrimination between the immunogenicity of the rBCG strains.

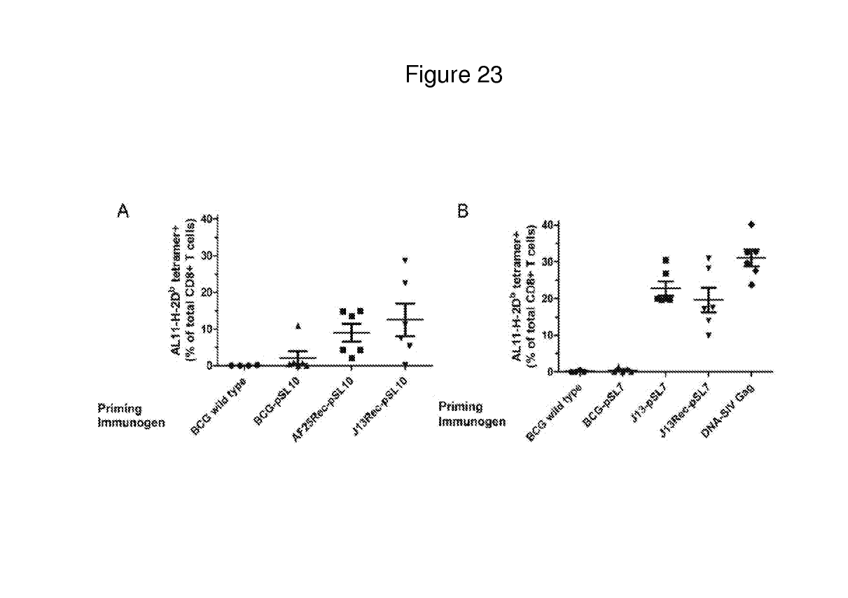

No Gag-specific CD8+ T cell response was observed in mice primed by wild type BCG Danish and boosted with this low dose of rAd5-Gag (FIGS. 2. 10A). A mean AL11 tetramer response of 2.2% was observed in mice primed with wild type BCG-SIV Gag and boosted with rAd5-SIV Gag. Importantly, we observed a significant increase in the boosted tetramer responses in mice primed with the novel constructs J13Rec-pSL10 and AF25Rec-pSL10 compared to mice primed with the parental BCG Danish-pSL10 (FIG. 23A). AF25Rec-pSL10 primed for a response that was more than 4 times greater than the response primed by the parental rBCG strain, approximately 9% of the total peripheral CD8+ T cells. While the boosted response primed for by J13Rec-pSL10 showed much larger variation, the magnitude of the mean response was over 5 times greater than that of the parental construct, wild type BCG-pSL10, with 12.5% of all CD8+ T cells in the peripheral blood specific for the AL11 epitope.

We also wanted to compare the priming ability of these novel constructs with the well-established priming immunogen plasmid DNA. Mice were immunized with J13Rec-pSL7, the transposon mutant strain J13 transformed with pSL7, and a plasmid DNA vaccine; all mice were then boosted with rAd-SIV Gag (FIG. 23B). Boosted responses to J13Rec-pSL7 and to J13-pSL7 were of comparable magnitude (mean responses of 22.8% and 19.6%), and both were significantly greater than the mean response to the parental, unmutated BCG Danish-pSL7 (0.5%). These responses were of the same order of magnitude as the boosted response primed by a plasmid DNA vaccine encoding SIV Gag (31%).

### Discussion

The development of technologies for expressing foreign transgenes in mycobacteria has opened the door to using mycobacterial vaccines to prevent infections by pathogens other than tuberculosis. First generation rBCG vaccines were created using wild type BCG expressing selected antigens of diverse pathogens, and these first generation vaccines were tested in murine, nonhuman primate, and human studies. While the results demonstrated that rBCG can prime for a strong boost response to some of these antigens, we felt that the immunogenicity of the first generation rBCG strains might be increased through genetic manipulation of the mycobacteria.

In the present study, we have implicated 17 genes or operons of BCG in reducing antigen presentation and used this information to make a series of second generation rBCG vaccines. Two of these genes were selected for further evaluation. We employed site-directed mutagenesis using specialized transduction to recreate these mutations in wild type BCG, and the novel strains created through this work were compared to BCG Danish and plasmid DNA for their ability to induce CD8+ T cell responses in vivo. The novel reconstructed strains, termed J13Rec and AF25Rec, were more immunogenic than the parental strain of BCG and were comparable in terms of immunogenicity to a plasmid DNA vaccine. Thus we formally confirmed the importance of these two genes in rBCG immunogenicity, showing that the immunogenicity of these two novel strains was dramatically increased over the immunogenicity of the parental strain of BCG. Importantly, we also showed that these novel strains of rBCG elicited an immune response comparable to that induced by the well-established priming immunogen plasmid DNA.

Previous screens of mycobacterial mutant libraries have identified genes essential to mycobacterial growth, pathogenicity, and host infectivity. While these investigations have yielded important fundamental information on the biology of the bacterium and have identified strains that confer some protection against M. tuberculosis, they have not yet been translated into a viable clinical product. The screen chosen for the present work had an endpoint of increased MHC class I presentation and increased MHC class I-restricted T cell responses. The choice of this endpoint of increased immunogenicity allows the findings of the screen to be immediately translated into a vaccine setting.

In the present work, 17 novel BCG transposon mutant strains have been identified that have increased MHC class I antigen presentation. From the sequence data, 15 distinct genes were identified in the BCG genome that contribute to modulating antigen presentation; in two cases independent mutations mapped to the same operon. These 15 distinct genes have been shown to map to 13 various sites in the genome with no evidence of clustering. This is consistent with their diverse predicted functions, and this diversity speaks to the numerous pathways through which BCG can modulate the immune response.

While all of these genes have been implicated in modulating antigen presentation in this screen, the particular pathways in which these genes function have not been elucidated. The products of these genes may be effector proteins modulating the host's immune response, or these gene products may act upstream in pathways that create effector proteins, mediating the regulation of DNA transcription, translation, protein modification or secretion of effector proteins.

The 15 defined genes can be divided into three groups. The first group contains genes that have an effect linked to modulating the immunogenicity of mycobacteria. Members of the PPE gene family have been implicated in immune modulation, and changes in cmaA2 have been implicated in macrophage activation. A second group includes genes that may function by an indirect mechanism, most likely upstream in the pathways that produce effector proteins. This group includes genes that encode transcription factors, chaperoning proteins, kinases and proteins active in metabolism. Finally, a third includes those proteins with no known function or homology to known genes.

The transposon mutagenized rBCG strains themselves cannot be used as vaccines in human trials. The persistence of a transposon and an antibiotic resistance gene in each of these constructs strains poses a significant health risk for humans. To begin to address these issues, novel strains of BCG were created using allelic exchange delivered via specialized transduction. This strategy allowed for the introduction of a hygromycin antibiotic resistance marker in place of cmaA2 in the strain J13Rec and the hygromycin antibiotic resistance cassette in place of the operon containing BCG_{—}2587-BCG_{—}2590 in AF25Rec. Gamma delta resolvase sites flanking the hygromycin cassette will allow future unmarking of the strains. This method avoids the use of a potentially unpredictable transposon and employs antibiotic selection in such a fashion that it can be eliminated through future manipulation of these micro-organisms.

The gene cmaA2 and the operon BCG_{—}2587-BCG-2590 were selected for deletion in wild type BCG because the strains J13, AF25, and C57 generated particularly strong and consistent prime and boost immune responses in vivo. CmaA2-deficient strains of M. tuberculosis have been shown to generate greater inflammation and innate responses through increased macrophage activation as compared to wild type M. tuberculosis [178]. The increased innate responses and increased macrophage activation may be responsible for increased peptide presentation to T cells. CmaA2 exists in an operon, and it is possible that the transposon has a disruptive effect on expression of the upstream gene in the operon, BCG_{—}0547c, which has putative enoyl-CoA hydratase activity. Much less is known about the genes deleted in the AF25 and C57 constructs. The four-gene BCG operon containing BCG_{—}2587, BCG_{—}2588, BCG_{—}2589, and BCG_{—}2590, is homologous to the three-gene M. tuberculosis operon that includes Rv2565, Rv2566, and Rv2567. The first gene of the operon, BCG_{—}2587, is homologous to Rv2565, and has a putative cyclic AMP receptor protein effector domain. A point mutation in Rv2566 is associated with expression of the protein in BCG in two fragments, as BCG_{—}2588 and BCG_{—}2589. BCG_{—}2588 and BCG_{—}2587 have transglutamine like-enzyme domains and putative amidoligase enzyme domains. BCG_{—}2590 has no known function. While our data indicate that this operon plays a role in immunogenicity, it is unclear how these genes function in this role.

Site-directed disruption of genes in slow-growing mycobacteria is a complex procedure. Because of the slow growth kinetics of BCG, it takes several weeks to expand a population of this bacterium with a genetic modification. To disrupt a specific gene, an AES must be created that will catalyze crossing over between homologous arms that flank the gene of interest, resulting in the exchange of a hygromycin cassette for the gene of interest. Unlike modifying fast growing mycobacteria, where electroporation can be used to introduce an AES with a high rate of homologous recombination, electroporation of an AES into slow growing mycobacteria results in a high rate of illegitimate recombination. Homologous recombination does not occur, and the hygromycin cassette is inserted randomly into the genome. Specialized transduction, using a temperature sensitive phage identified by Jacobs and his colleagues [170] delivers the AES in such a way as to reduce substantially the rate of illegitimate recombination. Nevertheless, illegitimate recombination still occurs at a low rate using specialized transduction. For this reason, we developed a number of PCR primers specific for the genes of interest to screen colonies and cultures for the deletion of interest. Southern blotting was ultimately used to confirm the PCR results once a novel strain was created.

Some genes may be essential for mycobacterial growth, and it may be impossible to delete them and retain a viable bacterium. Deletion of the cmaA2 gene has been attempted in BCG with limited success. CmaA2 is a mycolic acid methyltransferase that is not critical for M. tuberculosis in vitro growth [178]. BCG strains obtained after 1927 lack other methyltransferases that have functionally similar activities, e.g. mmaA3 in BCG Pasteur and BCG Danish contains a G→A point mutation leading to a glycine→aspartic acid substitution [24]. Because of the absence of a functional mmaA3 in BCG Pasteur, it has been reported that cmaA2 cannot be deleted without loss of oxygenated mycolates and loss of cell viability [179]. We sequenced the mmaA3 gene of the BCG Danish strain as well as the two novel strains, J13Rec and AF25Rec. Our sequence data indicate that all three strains contain a 293_{G}_{_{→}}_{A} base pair substitution within the mmaA3 gene, consistent with previous reports indicating that the mmaA3 gene is disrupted. However, after two attempts at transducing BCG Danish with the temperature sensitive phage containing an AES specific for cmaA2, we were able to obtain two colonies, both of which had the cmaA2 gene deleted.

To assess in vivo T cell responses, the new constructs J13Rec and AF25Rec were transformed with a SIINFEKL-expressing plasmid. Expression of the transgene from the new constructs was equivalent to expression from BCG Danish transformed with the same plasmid, yet the immune responses generated to J13Rec-SIINFEKL and AF25Rec-SIINFEKL were much more robust. The increased immunogenicity of these constructs was a result of the gene disruption caused by allelic exchange, which did not affect transgene expression.

We wanted to determine whether J13Rec and AF25Rec could vector antigens other than SIINFEKL that would be more representative of antigens that might expressed by a vaccine construct. The chicken ovalbumin MHC class I restricted epitope SIINFEKL is one of the most immunodominant epitopes studied and was therefore suited for study as a model immunogen in vivo. However, many vaccine antigens are weaker immunogens than SIINFEKL. We therefore introduced the SIV gag gene into the mutant mycobacterial strains on two plasmids that differed in the secretion signal directing export of the SIV Gag antigen. When vectored by mycobacteria, primary tetramer responses to the SIV Gag AL11 epitope were undetectable in mice. For this reason, we chose to discriminate between the immunogenicity of the BCG strains based upon the immune response following boosting with Ad5-SIV Gag. SIV Gag-specific boosted responses in the J13Rec- and AF25Rec-primed mice were significantly higher than those primed with the recombinant unmutated parental strain of BCG, consistent with the data generated in the SIINFEKL system. These findings indicated that this phenotype of increased immunogenicity was not unique to BCG strains vectoring the SIINFEKL epitope.

Most importantly, the priming of SIINFEKL-specific and SIV Gag-specific CDS+ T cell responses elicited by the J13Rec and AF25Rec rBCG strains were comparable to the priming of responses to plasmid DNA. This is significant because plasmid DNA vaccines are established successful priming immunogens. However, mycobacterial vaccines are less expensive, easier to produce, and their safety profile is already accepted worldwide as 2 billion people are already immunized with BCG.

In summary, we have identified a number of genes that rBCG uses to modulate antigen presentation and T cell responses, and harnessed this information to create novel vaccine constructs. Mycobacterial vectors induce a very strong CD4+ T cell response, and prime for augmented antibody responses when delivered prior to protein immunogens. The two novel vaccine vectors described in these studies generated increased antigen presentation and increased MHC class I-restricted CD8+ T cell responses. It is possible that this increased presentation is also associated with better MHC class II-restricted T helper cell and antibody responses.

### IV. Discussion

M. bovis BCG has a number of properties that make it an attractive vaccine vector. Among those properties are its ability to generate a robust CD4+ T cell response, a Th1 cytokine profile, a transgene product-specific antibody response, and a transgene product-specific CD8+ T cell response. However, the magnitude of transgene product-specific CD8+ T cell responses observed following vaccination with rBCG immunogens has been disappointing. The generation of a transgene product-specific CD8+ T cell response is determined in large part by the nature of the immune response induced by the vector. Therefore, it was our goal to increase the transgene product-specific CD8+ T cell responses generated by rBCG by creating a more immunogenic vector. The work described here seeks to increase the transgene product-specific CD8+ T cell response to BCG through identifying genes that suppress T cell responses and then eliminating these genes from BCG.

Assessment of T Cell Responses to rBCG Vaccines

The ability to assess transgene product-specific CD8+ T cell responses has been facilitated by multicolor flow cytometry analysis of tetramer-stained T cells. Tetramer technology has provided the field of immunology a sensitive assay for quantifying epitope specific CD8+ T cell responses, allowing the head-to-head comparison of vaccines. In the studies performed here, tetramer analysis of T cells was performed on lymphocytes isolated from the peripheral blood of mice. There are limitations in evaluating a vaccine by examining only a single anatomic compartment; however, screening large numbers of mutants required a standard assay, and for the purposes of comparing large numbers of vectors in a controlled manner we chose to examine systemic T cell responses through tetramer staining of the peripheral blood.

We chose to assess tetramer responses to three different immunodominant epitopes vectored by BCG: the SIINFEKL epitope from chicken ovalbumin, the AL11 epitope from SIV Gag, and the p18 epitope from HIV Env. First generation rBCG vectors expressing the SIINFEKL and p18 epitopes generated limited tetramer responses in the peripheral blood of mice, with mean responses ranging from 0.2-0.8% of total peripheral blood CD8+ T cells in mice. Tetramer responses to the SIV Gag AL11 epitope by first generation rBCG vaccines are undetectable. First generation rBCG vaccines vectoring a wide range of antigens have been tested in nonhuman primate and human clinical studies, but have failed to demonstrate robust transgene product-specific immunogenicity.

In order to increase transgene product-specific immune responses, a number of second generation recombinant mycobacterial vectors were developed through rational genetic modification. These vectors, however, have not generated significantly greater tetramer+ CD8+ T cell responses in the peripheral blood than those generated by first generation unmodified rBCG vaccines. A modified RD1 deletion M. tuberculosis vaccine with a secA2 deletion has been developed to vector the HIV Env protein. CD8+ T cells specific for the p18 epitope in the peripheral blood of vaccinated mice ranged from 0.15-0.2% of total CD8+ T cells, a response that was less than the response to unmutated rBCG expressing the same Env construct. This secA2 deletion, reproduced in M. bovis BCG rather than M. tuberculosis, did not increase the p18 tetramer+ CD8+ T cell response induced by rBCG-ΔsecA2-HIV Env in mice. [180]. A different second generation modified rBCG vaccine, 3 DBCG, which expresses less superoxide dismutase (SodA), generates increased tetramer responses specific for endogenous proteins such as TB10.3/10.420-28 (GL9). While tetramer responses to endogenous proteins are increased, increased tetramer+ CD8+ T cell responses against a heterologous antigen delivered by this vector have not been demonstrated [181]. Second generation modified rBCG vaccines with leuD deletions and other auxotrophic mutations have failed to generate increased transgene product-specific CD8+ T cell responses. A lysine auxotrophic strain of BCG vectoring HIV Env elicited no detectable Env-specific T cell responses following inoculation in mice [182]. This finding suggests that auxotrophic deletions increase the safety profile of a rBCG vector but do not improve its immunogenicity.

The AERAS 401 strain of rBCG contains a rational genetic modification that allows transgenic proteins access to the cytosol and the traditional MHC class I processing machinery. The perfringolysin gene was inserted in the ureaseC gene of the AERAS 401 strain of BCG, giving it the ability to puncture holes in the phagosome, allowing egress of the transgenic proteins from the phagosome containing the recombinant mycobacteria. We obtained this second generation rBCG strain, expressed SIINFEKL from it, and assessed SIINFEKL-specific CD8+ T cell responses in mice. We observed no vaccine-associated increase in SIINFEKL tetramer+ CD8+ T cell responses in mice; in fact, responses to AERAS 401-SIINFEKL were on average lower than responses to the parental strain of rBCG-SIINFEKL. Similarly, in nonhuman primates studies, limited or no detectable primary tetramer positive CD8+ T cell responses have been demonstrated in response to the AERAS 401 strain vectoring HIV Env [159].

Rationale for Novel Second Generation rBCG Vaccines Generated by Random Mutagenesis

The limited success of rationally designed second generation rBCG vaccines suggested that a different approach for modifying rBCG vectors may be necessary. We therefore undertook a study to identify BCG genes that suppress the host CD8+ T cell response and used the information generated in the study to create novel rBCG vectors. This approach to the creation of a genetically modified rBCG vaccine is fundamentally different from the approach previously used to build such strains. Rather than making rational genetic changes based upon findings published in the literature, we chose to employ random mutagenesis and a screen to identify potentially useful changes. This screen identified changes by directly testing immunogenicity. Therefore, the information obtained through this screening effort was immediately translatable into a novel rBCG vector with increased immunogenicity.

Mutants that Generated Increased Immune Responses in the Screen

The screen that we applied was two tiered; it had both an in vitro and an in vivo component. We utilized an in vitro MHC class I presentation assay to assess transgenic epitope presentation to CD8+ T cells by macrophages infected with transposon mutant strains of rBCG. From a library of 3290 rBCG mutants that was screened, 122 strains generated increased presentation of the SIINFEKL epitope in infected macrophages. Of these 122 mutants that generated increased in vitro responses, 76 were tested for their ability to generate increased transgene product-specific CD8+ T cell responses in vivo; of which 37 strains generated responses greater than the responses generated by the parental strain. Finally, when boosted with a heterologous recombinant vector, 17 of these 37 strains generated secondary transgene product-specific CD8+ T cell responses that were greater than the response generated by the parental, unmutated strain of rBCG.

The in vitro tier of the screen had a positive rate of 3.7% ( 122/3290). The in vivo tier had a positive rate of 50% ( 37/76) for mutants generating an increased primary CD8+ T cell response. Twenty percent ( 17/76) of strains generating an increased primary CD8+ T cell response also primed for an increased heterologous boost response. Thus, the effect of the two tiered screening approach was a positive rate of 0.8% for identifying mutant strains with increased primary and secondary CD8+ T cell responses. BCG has approximately 4000 potential protein-coding reading frames. Of these genes, approximately 600 have been identified as essential to growth, and transposon disruption of the genes leads to a non-viable bacterium. Furthermore, the mariner transposon is specific for the TA dinucleotide, and less than 10 genes within the BCG genome lack TA dinucleotides [183]. Therefore, between 3000 and 3400 genes are potential targets for transposon disruption. As we have analyzed 3290 random transposon mutants and multiple clones may have disruptions in the same gene, we have not performed a saturating analysis of all potentially disruptable genes. Nevertheless, we found several instances where two independent rBCG strains generating increased immunogenicity had disruptions that mapped to the same gene or operon. This redundancy suggests that there are a limited number of pathways that modulate CD8+ T cell responses specific for the transgene product, and indicates that we have approached the limit of identifying all of these pathways.

Mutants that Did not Generate Increased Immune Responses in the Screen

Approximately 51% of the rBCG mutants generating increased presentation in vitro did not generate increased transgene product-specific CD8+ T cell responses in vivo. The generation of a CD8+ T cell response is complex, requiring a TCR/MHC:peptide interaction as well as costimulatory, secondary, and cytokine signals. Many hybridomas do not require all of the secondary signals for activation in an in vitro setting, and while the RF33.70 line has been shown to require CD8 costimulation for activation, its requirement for activation based on secondary stimulation signals such as B7.1 and B7.2 may not be as stringent as a CD8+ T cell in vivo [161]. Therefore, it is possible that a mutant rBCG strain infecting a macrophage may trigger a signal leading to hybridoma activation that would not lead to the activation of a true CD8+ T cell. The rBCG mutants that generated increased presentation in vitro but did not induce increased tetramer responses in vivo may have been false positives because of the use of a hybridoma line for the in vitro screen. Alternatively, some of the strains that increased presentation in vitro may have done so through a mechanism that is not a limiting factor in the generation of CD8+ T cell responses in vivo.

Several rBCG strains generated increased primary transgene product-specific CD8+ T cell responses without priming for increased heterologous boost responses. The generation of an enhanced CD8+ T cell response without sufficient CD4+ T cell help may explain this observation. Fifteen strains of rBCG generated increased primary responses and primed for increased heterologous boost responses when immunized with rAd-SIINFEKL. The transgene product-specific CD8+ T cells primed by these strains may have been induced in the presence of increased levels of CD4+ T cell help, resulting in a more robust memory cell response.

Identification of Gene Disruptions in Select Transposon Mutant rBCG Strains

The genes disrupted by the transposon insertions in the 15 strains that elicited increased transgene product-specific CD8+ T cell responses were sequenced. These sites mapped to 13 unique genes or operons; in two cases there were two strains with disruptions in the same operon. Surprisingly, there was very little similarity in predicted function of the identified genes. Also noticeable was that we identified genes at multiple stages of protein production, including regulators of transcription of DNA, protein modification, and protein secretion. For the purpose of analysis, the 15 genes could be divided into three different categories based upon their predicted functions: i) those genes whose products act directly on the immune system, termed effector proteins; ii) those genes whose products modify or impact the production and secretion of an effector protein; and iii) those genes whose products have no clear link to immunogenicity, including genes with unidentified functions.

Infection of macrophages by mycobacteria has been shown to have a direct apoptotic effect upon specific, and to an even greater extent, nonspecific T cells [177]. Effector molecules expressed by pathogenic mycobacteria may have a direct effect upon the macrophage or T cell and act to decrease T cell responses. The disruption of several of the genes identified in this screen, including those encoding such proteins as LprG, PDIM, and PPE41 proteins, prevent the expression of effector molecules. LprG has previously been shown to have an effect upon MHC class II presentation through its interaction with TLR-2; PDIM insertion into the phagocytic membrane has been shown to arrest phagosomal maturation; and PPE proteins have been implicated in pathogenicity, although their function and mechanism of action remain unclear. Genes encoding the Proline-Glutamic Acid (PE) and the Proline-Proline-Glutamic Acid (PPE) motif comprise a surprisingly large amount of the genome of pathogenic mycobacteria, close to 10%, and are not present in other bacteria [184]. Therefore, it is possible that PPE41 may be a direct effector protein, as it has been implicated in modulating the CD8+ T cell response specific for the transgene product in our work, and has been show to be secreted through the Esat-1-like secretion system Esx-5 [185].

Several genes identified in this study encode proteins predicted to modify other molecules. These include an efflux pump, amidoligase, acyl-CoA ligase, hydratase, cyclopropanase, and an isomerase. While not effector molecules themselves, these gene products could be enzymes functioning to modify final effector molecules. Transcription factors were also implicated in this investigation, and these gene products may play a role in regulating expression of effector molecules. Several identified genes encode products that have no clear role in the production or expression of effector proteins. Rather, they have putative cellular functions. These include an oxidoreductase, critical in nitrogen metabolism, a chaperoning protein, and an ATP-dependent DNA ligase. These proteins may act to change the cellular state resulting in the evasion of the immune system.

Gene Clustering

With 13 different genetic loci implicated by this mapping of transposon insertion sites, we were interested to determine whether these genes were located in pathogenicity islands. Pathogenic islands of genomic DNA span 10-20 kb and encode multiple genes that were introduced into a genome often as a result of horizontal gene transfer. The gene mgtC was first identified in Salmonella enterica and was shown to confer a survival advantage within macrophages; this same gene was later identified in M. tuberculosis and shown to confer the same function. This gene does not exist in closely related strains of mycobacteria [186]. Similarly, a number of toxin/antitoxin systems are present in the M. tuberculosis genome within organized pathogenicity islands, and these genes are also not present in closely related mycobacterial species. These genes were likely acquired by horizontal gene transfer [187, 188]. Therefore, we wanted to determine whether the genes identified in this work that were all associated with the same phenotype of modulation of CD8+ T cell responses were similarly organized. When we mapped these 13 loci we observed no evidence of clustering or pathogenic islands. This finding is consistent with a wide range of predicted functions for the genes implicated in the present study. Many of these genes regulate or modify end product effector molecules. Because they act in a trans manner, it is possible that they are located distant from the genes that encode the effector molecules.

Vaccine Creation

Through this two-tiered screening process we have implicated 15 genes at 13 independent genetic loci that can modulate the transgene product-specific CD8+ T cell response. With the endpoint of this screen being increased CD8+ T cell responses, the disruption of the genes identified in this work can be immediately translated into the creation of more immunogenic vaccine vectors.

Initially, we attempted to recreate the gene deletion defined in the A25 transposon strain. We used specialized transduction to disrupt the gene BCG_{—}2384c, identified in this strain, and were successful at both gene deletion and unmarking the strain. However, we were unable to introduce any foreign DNA encoding a transgene into the BCGΔ2384c strain despite multiple attempts. We then attempted to recreate deletions identified in other strains. We identified the gene cmaA2 as being disrupted in the J13 transposon mutant strain, and we used specialized transduction to delete the cmaA2 gene in BCG Danish. This mutation was consistently associated with responses several fold greater than the response generated by the parental strain.

Significance of the J13 Transposon Disruption and the J13Rec cmaA2 (BCG_{—}0546c) Deletion

The gene cmaA2 has been examined in studies unrelated to vaccination, and plays a critical role in virulence and shaping the immune response to pathogenic mycobacteria. This gene was originally identified as coding for a cyclopropane synthase through its homology with the M. leprae cmaA1 gene. CmaA2 in M. tuberculosis and BCG has been shown to modify the cell envelope, a critical virulence determinant in mycobacterial infection [189]. One of the main components of the mycobacterial cell envelope is mycolic acid, a molecule unique to the genus Mycobacterium and related taxa, and can be found in both pathogenic and saprophytic strains of mycobacteria. These mycolic acids are α-alkyl, β-hydroxy fatty acids between 75 and 85 carbons in length. They can be found as part of trehalose dimycolate (TDM) or esterfied to peptidoglycan linked arabinogalactan [190].

Modification of mycolic acids occurs in all mycobacteria; however, only pathogenic mycobacteria are capable of mycolic acid cyclopropanation [189]. The fact that mycobacteria encode a number of S-adenosyl methionine dependent methyl transferases that modify mycolic acid with methyl branches and cyclopropane rings while E. coli encodes only a single cyclopropane fatty acid synthase (CFAS) suggests the significance of these modifications for bacterial virulence. Cyclopropanation changes a double bond into a cyclopropane group, a change that confers resistance to treatment with hydrogen peroxide. As it only occurs in pathogenic mycobacteria, it may be an evolutionary adaptation to the harsh reactive oxygen species found in macrophage phagosomes that crosslink and degrade mycolic acids with exposed double bonds [191].

Three forms of mycolic acids are found in M. tuberculosis and M. bovis: alpha-, keto-, and methoxy-mycolates. A 293_{G}_{_{—}}_{A} point mutation found in the gene mmaA3 of M. bovis BCG strains obtained from the Pasteur institute after 1931 has eliminated methoxymycolate production [24]. In M. bovis BCG Pasteur and Danish, only alpha and ketomycolates are formed. CmaA2 catalyzes the formation of cis and trans proximal cyclopropane groups on oxygenated mycolates (keto and methoxy), although the formation of proximal cis cyclopropane groups is also mediated by MmaA2. Therefore, in M. bovis BCG Danish, CmaA2 has a unique role in adding the cyclopropane group to ketomycolates; in the absence of CmaA2, ketomycolates are formed without a proximal trans cyclopropane group [192].

The proximal trans cyclopropanation modification catalyzed by CmaA2 directly modulates cytokine production by the host during M. tuberculosis infection. Strains of M. tuberculosis containing a cmaA2 deletion generate 2-3 fold more TNF-α during infection of mouse bone marrow derived macrophages. These data and other findings suggest that the trans cyclopropanation modification catalyzed by CmaA2 actively suppresses TNF-α production early in infection [178]. These studies indicate that increased cytokine production was elicited by TDM containing mycolic acids lacking proximal trans cyclopropanation, acting in a MyD88-dependent but TLR-2- and TLR-4-independent mechanism [193]. These observations suggest that the cytokine profile generated in response to mycobacteria with a cmaA2 gene deletion may be triggered through an unidentified pattern recognition receptor for TDM.

Strains of cmaA2 deficient M. tuberculosis have been found to be hypervirulent. Increased pathology, increased granuloma formation, and a shortening of the mean time to death of 320 to 227 days following infection were all a consequence of cmaA2 deletion. However, this pathology may be due to the hyperactivation of the immune response mediated by increased cytokine responses, as evidenced by the fact that no increased death or pathology was observed in IFN-γ−/− and TNF-α−/− mice. Importantly, increased pathology caused by cmaA2 deficient M. tuberculosis was not a result of increased bacterial burdens as there were no differences in M. tuberculosis and M. tuberculosis ΔcmaA2 burdens in the spleens and livers of two strains of infected mice [178].

Our data indicate that deletion of the cmaA2 gene in BCG results in infected macrophages increasing MHC class I presentation of transgenic proteins and increased generation of transgene product-specific CD8+ T cell responses. This observation is consistent with previous observations of the effect that cmaA2 deletion has on M. tuberculosis. It is possible that increased levels of TNF-α and other early acting Th1-skewing cytokines are induced in response to ketomycolates of BCG lacking proximal trans cyclopropanation. The increased levels of these cytokines may set the stage for an increased state of macrophage activation, leading to increased presentation of transgenic epitopes to T cells. This mechanism would suggest that the increased presentation of epitopes is not restricted to MHC class I-presented epitope peptides; it is quite possible that more robust CD4+ T cell responses are also being generated. Our data demonstrating more robust primary as well as secondary memory CD8+ T cell responses in response to J13Rec is consistent with the prevailing theory that the generation of a CD8+ T cell memory response requires CD4+ T help, and it is possible that BCGΔcmaA2 stimulates greater levels of both MHC class I and MHC class II presentation. The transposon disruption in cmaA2 may also have a polar effect on the upstream gene in the operon, BCG_{—}547c, an cnoyl-CoA hydratase. The transposon mutant AZ11 also has putative enoyl-CoA hydratase activity, suggesting that this family of enzymes may play a role in shaping the immune response to slow growing mycobacteria.

Significance of the AF25/C57 Transposon Disruptions and the AF25/C57Rec Deletion

We also chose to disrupt the BCG_{—}2587-2590 operon, identified in the AF25 and C57 mutants, using specialized transduction. Very little work has been done on the BCG_{—}2587-2590 operon and its homologous operon Rv2565-2567 in M. tuberculosis. We have identified two transposon disruptions in this operon that increased transgene product-specific T cell responses. Because of polar effects caused by a disruption of the operon, any or all of the genes of this operon may affect the immune response generated to rBCG. Domains contained within the various proteins encoded by this operon would suggest that the encoded proteins interact and function in a common pathway.

The first gene of the operon, BCG_{—}2587 (Rv2565), contains both the effector domain of the Cyclic AMP Receptor Protein (CAP) family of transcription proteins as well as a phospholipase domain. The transposon in the rBCG strain AF25 disrupts the second gene of the operon, BCG_{—}2588. This gene product contains a transglutaminase enzymatic domain at the N-terminus, and a putative amidoligase enzymatic domain at the C terminus. The third gene of the operon, BCG_{—}2589, disrupted by the transposon present in the strain C57, contains an amidoligase domain. BCG_{—}2588 and BCG_{—}2589 are expressed as independent genes in BCG, but in M. tuberculosis a base pair difference causes both genes to be expressed as one single protein. The amidoligase domain of the protein encoded by Rv2566 has been identified as a circularly permutated novel form of the COOH—NH2 ligase family of enzymes through in silico investigation by Lyer et al. [194]. Amidoligase family enzymes may play a significant role in the generation of immune responses to mycobacteria. In eukaryotic cells, amidoligase-like enzymes perform the critical function of ubiquitination of proteins targeting them for the proteosome. Recent studies have identified similar amidoligase function in prokaryotic cells; this function results in the addition of a Pup modification to proteins that destabilizes them [195]. Destabilization and degradation of proteins is critical for the generation of a cellular immune response, as the MHC class I and II machinery rely on small peptide fragments to present to T cell receptors, and these fragments often come from degraded proteins. The absence of amidoligase function may result in a different repertoire of peptides being presented to T cells. Amidoligase function can also modify proteins by adding functional groups to precursor molecules to hide or suppress highly immunogenic domains on precursor molecules. In the absence of amidoligase function and the final modifications, these precursor molecules are still made and may be highly immunogenic because their immunogenic domains are not hidden. The last gene of the operon, BCG_{—}2590 (Rv2567), contains 4 domains, the function of two of which are unknown. The other two domains have been recently identified as unique alpha helical domains and termed Alpha-E domains. It is thought that these domains interact with the ATP grasp and COOH—NH2 ligase of the other proteins in the operon [194].

Therefore, the increase in immunogenicity associated with the AF25 and C57 transposon disruptions may be a result of the loss of activity of a transcription factor, a phospholipase, an amidoligase, a protease, or any combination of these. Because the transcription factors and enzymes encoded by the operon BCG_{—}2587-2590 have the potential to interact, it is possible that they have a coordinated function to produce and modify an effector molecule that has an impact on the generation of CD8+ T cell responses.

Comparison with Other Vaccine Vectors

In the studies presented here, we examined the transgene product-specific CD8+ T cell response induced by the modified rBCG vaccine AERAS 401, a DNA vaccine, a first generation rBCG construct, and the novel second generation rBCG constructs. We found that the AERAS 401 construct generates a transgene product-specific CD8+ T cell response no different from that induced by the parental first generation rBCG vector. The second generation rBCG vaccines J13Rec and AF25Rec that we have created generated tetramer responses upon priming that were several times greater than those generated by first generation and AERAS 401 constructs. We observed a three- to four-fold increase in the transgene product-specific CD8+ T cell response generated by the two novel constructs J13Rec and AF25Rec over those generated by the parental strain of BCG.

We have also examined the transgene product-specific CD8+ T cell response following heterologous boost immunization with a suboptimal dose of recombinant adenovirus. Boosted responses in mice primed with the first generation rBCG vaccine and AERAS 401 were significantly lower than the boosted responses in mice primed with the two novel constructs J13Rec and AF25Rec. Importantly, a plasmid DNA vaccine, known to prime for a very robust immune response in animal models, primed for immune responses that were comparable in magnitude to those primed for by the two novel rBCG constructs J13Rec and AF25Rec.

These results demonstrate the effectiveness of a two tiered screening approach, combining in vitro and an in vivo components, for the identification of mycobacterial genes that suppress the immune response. The screen has yielded information on a number of genes that will open up investigation into their function in both BCG and M. tuberculosis. By investigating these genes in the setting of pathogenic M. tuberculosis infection, novel pathways of immune suppression may be identified that will contribute to our understanding of how M. tuberculosis evades the immune system. Furthermore, investigation into pathways involved in the suppression of CD8+ T cell responses may give us a better understanding of the significance of the CD8+ T cell response and its role in the containment of pathogenic M. tuberculosis infections.

This work has also generated several novel BCG vector strains that generate increased CD8+ T cell responses against transgenic antigens. Two recombinant antigens have been expressed by the novel rBCG strains in this work and the vector has performed similarly for both; there is a good reason to believe that the J13Rec and AF25Rec strains will generate increased CD8+ T cell responses against other antigens. Therefore, these improved rBCG vectors could be applied to delivering antigens for vaccination against a variety of pathogens. In this work two loci were chosen for deletion, although 11 other loci were identified in the screening whose deletion may lead to other candidate vaccine vectors.

Furthermore, the J13Rec and AF25Rec constructs may be able to serve as vaccines or adjuvants without transgene inserts. Treatments for several cancers have involved the use of BCG because of its highly immunogenic nature. This mycobacterium has adjuvant properties that can overcome T cell tolerance against cancer immunogens [196, 197]. Adjuvants such as Freund's adjuvant contain mycobacterial cell wall components; by incorporating the mutations identified in this screen in the mycobacteria used in these adjuvants, it may be possible to generate more potent T cell stimulation.

The BCG strains generated in this work may also have an application as an improved tuberculosis vaccine. While the role of CD8+ T cells in controlling M. tuberculosis infection may not be clear, data generated from nonhuman primate CD8+ depletion studies indicates that a CD8+ T cell response may be able to control a tuberculosis infection. Macaques immunized with BCG controlled M. tuberculosis challenge through a CD8+ T cell mechanism, as evidenced by the loss of protection associated with antibody-depletion of CD8+ T cells [59]. Therefore, a BCG strain capable of increasing CD8+ T cell responses could confer improved control of M. tuberculosis. If this vector increases the magnitude and lifespan of the memory population of CD8+ T cells, it could overcome one of the limitations of BCG vaccination: protection can wane after approximately 10 years, and vaccinees immunized at birth are often susceptible to pulmonary tuberculosis as adults. Increasing the magnitude and lifespan of the memory CD8+ T cell response by use of the J13Rec or AF25Rec strains may afford protection against pulmonary tuberculosis in adults.

Finally, the improved CD8+ T cell responses observed in response to the novel rBCG constructs may be just one aspect of their increased immunogenicity. These constructs were selected based on a screen for increased antigen presentation to CD8+ T cells. However, the strains may generate increased antigen presentation to other components of the immune response, improving CD4+ T helper cell and antibody responses.

The work described here represents a critical step forward in modifying BCG to create a more immunogenic second generation vector that may be utilized as a vaccine vector for a variety of pathogens. We have attempted to address one of the fundamental limitations of BCG as a vaccine vector. We have described the use of two rBCG deletion mutants vectoring the SIV Gag protein to generate more robust SIV Gag-specific cytotoxic T cell responses, constructs that may be used in the creation of an HIV vaccine.

Antigenic rBCG Vectors of the Invention

Recombinant BCG (rBCG) vectors of the invention can include one or more transgenes (e.g., proteins or peptides for use as antigens) incorporated into a mycobacterial vector that includes a mutation in one or more of the sites identified herein as modulating CD8+ T-cell responses (e.g., a mycobacterial vector that includes a mutation that ablates function of one or more of the genes or operons described herein; the vector may also include mutations that ablate function in combinations of the genes (e.g., mutations in 2, 3, or 4 or more of the genes) identified herein). The mutation can include a deletion, substitution, or addition at the site of the gene(s) that reduces function of the encoded gene(s) or that reduces or prevents expression of the encoded gene(s). In another embodiment, the mutation is a deletion or substitution of all or a portion of the encoded gene(s) that reduces function of the encoded gene(s) or that reduces or prevents expression of a functional gene product or product(s).

For example, one or more proteins or peptides as antigens can be incorporated into a mycobacterial vector (e.g., incorporated into the genome of a mycobacterium or in a plasmid, such as a episomal plasmid, that is stably tranfected in the mycobacterium) having a mutation at the site of, e.g., one or more of the following genes: BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c (or the equivalent homolog(s) in another mycobacterial species, e.g., M. africanum, M. avium, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi), or at the site of the operon for one or more of these genes. In other embodiments, the BCG vector of the invention includes mutations in combinations of one or more of the following genes: BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c (or the equivalent homolog(s) in another mycobacterial species, e.g., M. africanum, M. avium, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi), or at the site of the operon for one or more of these genes. For example, the BCG vector may have one or more mutations that ablate expression, or reduce expression, of BCG_{—}0546c (J13) and one or more mutations that ablate expression, or reduce expression, of one or more of BCG_{—}0381, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c (or the equivalent homolog(s) in another mycobacterial species). In another embodiment, the BCG vector may have one or more mutations that ablate expression, or reduce expression, of BCG_{—}2588 (AK25) and one or more mutations that ablate expression, or reduce expression, of one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2589, BCG_{—}3231C, BCG_{—}3297, BCG_{—}3445, and/or BCG_{—}3808c (or the equivalent homolog(s) in another mycobacterial species). In yet another embodiment, the BCG vector may have one or more mutations that ablate expression, or reduce expression, of BCG_{—}1790 (K14) and one or more mutations that ablate expression, or reduce expression, of one or more of BCG_{—}0381, BCG_{—}0546c, BCG_{—}0992, BCG_{—}0993, BCG_{—}1472c, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c (or the equivalent homolog(s) in another mycobacterial species). In still another embodiment, the BCG vector may have one or more mutations that ablate expression, or reduce expression, of BCG_{—}3445 (AZ11) and one or more mutations that ablate expression, or reduce expression, of one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, and/or BCG_{—}3808c (or the equivalent homolog(s) in another mycobacterial species).

In another embodiment, the BCG vector may have one or more mutations that ablate expression, or reduce expression, of BCG_{—}3231c (BL2) and one or more mutations that ablate expression, or reduce expression, of one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3297, BCG_{—}3445, and/or BCG_{—}3808c (or the equivalent homolog(s) in another mycobacterial species).

In another embodiment, the BCG vector may have one or more mutations that ablate expression, or reduce expression, of BCG_{—}3808c and one or more mutations that ablate expression, or reduce expression, of one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231c, BCG_{—}3297, and/or BCG_{—}3445 (or the equivalent homolog(s) in another mycobacterial species).

In any of the BCG vectors described above, the entire gene or operon may be deleted or only a portion of the gene or operon may be mutated (e.g., by a substitution, insertion, or deletion of one or more nucleic acids in the gene) so long as the gene or operon is not expressed in the recombinant mycobacterial vector (e.g., the polypeptide(s) encoded by the gene or one or more polypeptides encoded by the operon is not expressed or is expressed in an inactive form or in a form having substantially reduced activity relative to the unmutated polypeptide(s); e.g., a reduction of at least 10% activity, more preferably a reduction of at least 20%, 30%, 40%, 50% activity, and most preferably a reduction of at least 60%, 70%, 80%, 90%, 95%, or more activity).

Alternatively, the rBCG vector of the invention may include one or more mutations (e.g., one or more deletions, substitutions, or insertions) that ablate or substantially reduce the level of expression of one or more genes or operons in the rBCG vector (e.g., one or more mutations at one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c, their operon(s), or their promoter region(s), or the equivalent homolog(s), operon(s), or promoter region(s), respectively, in another mycobacterial species). For example, the rBCG vector of the invention may include one or more mutations (e.g., one or more deletions, substitutions, or insertions) that reduce the level of expression of one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c, or the equivalent homolog(s) in another mycobacterial species, by at least 10%, more preferably at least 20%, 30%, 40%, 50%, and most preferably at least 60%, 70%, 80%, 90%, 95%, or more relative to the unmutated vector. The mutation in the rBCG vector may be a deletion of all or only a portion of one or more of the gene(s), operon(s), or their promoter region(s), so long as the gene(s) or operon(s) is not expressed in the recombinant mycobacterial vector or is expressed at a reduced level (and/or produces a polypeptide having a form with no or reduced activity), relative to a BCG vector having an unmutated gene(s), operon(s), or promoter region(s). Similarly, if the mutation is a substitution or insertion of one or more nucleic acid molecules within one or more of these genes, their operon(s), or their promoter region(s), the substitution or insertion preferably results in non-expression (or reduced levels of expression) of the gene(s) or operon(s), or the expression of a polypeptide(s) encoded by the gene(s) or operon(s) having no or reduced activity, relative to a BCG vector having an unmutated gene(s), operon(s), or promoter region(s).

These rBCG vectors can be used as a prophylactic or therapeutic vaccine to induce an immune response to the protein- or peptide-based antigen (e.g., an antigen from a pathogen, an antigen from a cancer cell, or an allergen-based antigen). The BCG vectors described herein can be modified to include, e.g., peptides or proteins (such as those described herein or known in the art) from known pathogens (for example, infectious agents such as viruses, bacteria, fungi, and parasites, such as those mentioned herein or known in the art). The antigen may be all or a part of a single full-length protein or a chimeric fusion between the antigen and another protein or fragment thereof. In addition, the rBCG vectors of the invention can be modified to include a polypeptide- or peptide-based antigen selected from an antigen associated with autoimmune disease, a cancer-specific antigen, an allergen-specific antigen, an infectious disease antigen selected from a bacterial, viral, parasitic, and fungal antigen, a cytokine, a chemokine, an immunoregulatory agent, or a therapeutic agent. A nucleic acid molecule encoding the polypeptide or peptide antigen can be incorporated in the rBCG vector at any site known to induce an immune response. For example, the nucleic acid molecule encoding the polypeptide or peptide antigen can be inserted within the genome of the rBCG vector, within one or more of the 15 gene or operon sites identified herein (e.g., one or more of BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c, or the equivalent homolog(s) in another mycobacterial species, e.g., in order to ablate or reduce the level of expression of one or more of the gene(s) or operon(s)), or within a plasmid stably transformed in the rBCG vector.

In an embodiment, the invention features a mycobacterial vector (e.g., a rBCG vector) having at least one mutation that ablates or reduces expression of, e.g., one or more genes selected from BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and/or BCG_{—}3808c, or the equivalent homolog(s) in a mycobacterial species other than M. bovis BCG, or the operon(s) that includes one or more of these genes.

Included in the present invention are rBCG vectors having any one or more of the following combinations of mutations that ablate or reduce the level of expression of the indicated genes (or their homolog(s) in other mycobacterial species): BCG_{—}0546c and BCG_{—}0381; BCG_{—}0546c and BCG_{—}0992; BCG_{—}0546c and BCG_{—}0993; BCG_{—}0546c and BCG_{—}1472c; BCG_{—}0546c and BCG_{—}1790; BCG_{—}0546c and BCG_{—}1964; BCG_{—}0546c and BCG_{—}2067c; BCG_{—}0546c and BCG_{—}2384c; BCG_{—}0546c and BCG_{—}2449c; BCG_{—}0546c and BCG_{—}2580; BCG_{—}0546c and BCG_{—}2588; BCG_{—}0546c and BCG_{—}2589; BCG_{—}0546c and BCG_{—}3297; BCG_{—}0546c and BCG_{—}3445; BCG_{—}1790 and BCG_{—}0381; BCG_{—}1790 and BCG_{—}0546c; BCG_{—}1790 and BCG_{—}0992; BCG_{—}1790 and BCG_{—}0993; BCG_{—}1790 and BCG_{—}1472c; BCG_{—}1790 and BCG_{—}1964; BCG_{—}1790 and BCG_{—}2067c; BCG_{—}1790 and BCG_{—}2384c; BCG_{—}1790 and BCG_{—}2449c; BCG_{—}1790 and BCG_{—}2580; BCG_{—}1790 and BCG_{—}2588; BCG_{—}1790 and BCG_{—}2589; BCG_{—}1790 and BCG_{—}3297; BCG_{—}1790 and BCG_{—}3445; BCG_{—}2384c and BCG_{—}0381; BCG_{—}2384c and BCG_{—}0546c; BCG_{—}2384c and BCG_{—}0992; BCG_{—}2384c and BCG_{—}0993; BCG_{—}2384c and BCG_{—}1472c; BCG_{—}2384c and BCG_{—}1790; BCG_{—}2384c and BCG_{—}1964; BCG_{—}2384c and BCG_{—}2067c; BCG_{—}2384c and BCG_{—}2449c; BCG_{—}2384c and BCG_{—}2580; BCG_{—}2384c and BCG_{—}2588; BCG_{—}2384c and BCG_{—}2589; BCG_{—}2384c and BCG_{—}3297; BCG_{—}2384c and BCG_{—}3445; BCG_{—}3445 and BCG_{—}0381; BCG_{—}3445 and BCG_{—}0546c; BCG_{—}3445 and BCG_{—}0992; BCG_{—}3445 and BCG_{—}0993; BCG_{—}3445 and BCG_{—}1472c; BCG_{—}3445 and BCG_{—}1790; BCG_{—}3445 and BCG_{—}1964; BCG_{—}3445 and BCG_{—}2067c; BCG_{—}3445 and BCG_{—}2384c; BCG_{—}3445 and BCG_{—}2449c; BCG_{—}3445 and BCG_{—}2580; BCG_{—}3445 and BCG_{—}2588; BCG_{—}3445 and BCG_{—}2589; BCG_{—}3445 and BCG_{—}3297; BCG_{—}0546c, BCG_{—}2588, and BCG_{—}0381; BCG_{—}0546c, BCG_{—}2588, and BCG_{—}0546c; BCG_{—}0546c, BCG_{—}2588, and BCG_{—}0992; BCG_{—}0546c, BCG_{—}2588, and BCG_{—}0993; BCG_{—}0546c, BCG_{—}2588, and BCG_{—}1472c; BCG_{—}0546c, BCG_{—}2588, and BCG_{—}1790; BCG_{—}0546c, BCG_{—}2588, and BCG_{—}1964; BCG_{—}0546c, BCG_{—}2588, and BCG_{—}2067c; BCG_{—}0546c, BCG_{—}2588, and BCG_{—}2384c; BCG_{—}0546c, BCG_{—}2588, and BCG_{—}2449c; BCG_{—}0546c, BCG_{—}2588, and BCG_{—}2580; BCG_{—}0546c, BCG_{—}2588, and BCG_{—}2589; BCG_{—}0546c, BCG_{—}2588, and BCG_{—}3297; and BCG_{—}0546c, BCG_{—}2588, and BCG_{—}3445, or the equivalent homolog(s) in a mycobacterial species other than M. bovis BCG, or the operon(s) that includes one or more of these genes.

In an embodiment, one or more of the rBCG vector(s) described above further includes at least one viral antigen integrated within the rBCG vector selected from an antigenic peptide from an adenovirus, retrovirus, picornavirus, herpesvirus, rotaviruses, hantaviruses, coronavirus, togavirus, flavirvirus, rhabdovirus, paramyxovirus, orthomyxovirus, bunyavirus, arenavirus, reovirus, papilomavirus, parvovirus, poxvirus, hepadnavirus, or spongiform virus. In other embodiments, the at least one viral antigen includes peptides from at least one of HIV, CMV, hepatitis A, B, and C, influenza; measles, polio, smallpox, rubella; respiratory syncytial, herpes simplex, varicella zoster, Epstein-Barr, Japanese encephalitis, rabies, flu, or cold viruses. Examples of viral antigens for use with the present invention include, but are not limited to, e.g., HIV, HCV, CMV, adenoviruses, retroviruses, and picornaviruses. Non-limiting examples of retroviral antigens include retroviral antigens from the human immunodeficiency virus (HIV) antigens, such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components, such as hepatitis C viral RNA; influenza viral antigens, such as hemagglutinin and neuraminidase and other influenza viral components; measles viral antigens, such as the measles virus fusion protein and other measles virus components; rubella viral antigens, such as proteins E1 and E2 and other rubella virus components; rotaviral antigens, such as VP7sc and other rotaviral components; cytomegaloviral antigens, such as envelope glycoprotein B and other cytomegaloviral antigen components; respiratory syncytial viral antigens, such as the RSV fusion protein, the M2 protein and other respiratory syncytial viral antigen components; herpes simplex viral antigens, such as immediate early proteins, glycoprotein D, and other herpes simplex viral antigen components; varicella zoster viral antigens, such as gpI, gpII, and other varicella zoster viral antigen components; Japanese encephalitis viral antigens, such as proteins E, M-E, M-E-NS 1, NS 1, NS 1-NS2A, 80% E, and other Japanese encephalitis viral antigen components; rabies viral antigens, such as rabies glycoprotein, rabies nucleoprotein and other rabies viral antigen components. See Fundamental Virology, Second Edition, eds. Fields, B. N. and Knipe, D. M. (Raven Press, New York, 1991) for additional examples of viral antigens. The at least one viral antigen may be peptides from an adenovirus, retrovirus, picornavirus, herpesvirus, rotaviruses, hantaviruses, coronavirus, togavirus, flavirvirus, rhabdovirus, paramyxovirus, orthomyxovirus, bunyavirus, arenavirus, reovirus, papilomavirus, parvovirus, poxvirus, hepadnavirus, or spongiform virus. In certain specific, non-limiting examples, the at least one viral antigen is a peptide obtained from at least one of HIV, CMV, hepatitis A, B, and C, influenza, measles, polio, smallpox, rubella; respiratory syncytial, herpes simplex, varicella zoster, Epstein-Barr, Japanese encephalitis, rabies, flu, and/or a cold virus.

In an embodiment, the invention features a rBCG vector that expresses at least one bacterial antigen. Bacterial antigens for use with one or more of the rBCG vaccine vectors disclosed herein include, but are not limited to, e.g., bacterial antigens, such as pertussis toxin, filamentous hemagglutinin, pertactin, FIM2, FIM3, adenylate cyclase, and other pertussis bacterial antigen components; diptheria bacterial antigens, such as diptheria toxin or toxoid, and other diptheria bacterial antigen components; tetanus bacterial antigens, such as tetanus toxin or toxoid and other tetanus bacterial antigen components; streptococcal bacterial antigens, such as M proteins and other streptococcal bacterial antigen components; gram-negative bacilli bacterial antigens, such as lipopolysaccharides and other gram-negative bacterial antigen components, Mycobacterium tuberculosis bacterial antigens, such as mycolic acid, heat shock protein 65 (HSP65), the 30 kDa major secreted protein, antigen 85A, and other mycobacterial antigen components; Helicobacter pylori bacterial antigen components; pneumococcal bacterial antigens, such as pneumolysin, pneumococcal capsular polysaccharides and other pneumococcal bacterial antigen components; haemophilus influenza bacterial antigens, such as capsular polysaccharides and other haemophilus influenza bacterial antigen components; anthrax bacterial antigens, such as anthrax protective antigen and other anthrax bacterial antigen components; rickettsiae bacterial antigens, such as rompA and other rickettsiae bacterial antigen component. Also included with the bacterial antigens described herein are any other bacterial, mycobacterial, mycoplasmal, rickettsial, or chlamydial antigens. Bacterial antigens may also be derived from any of the following: haemophilus influenza; Plasmodium falciparum; neisseria meningitidis; streptococcus pneumoniae; neisseria gonorrhoeae; salmonella serotype typhi; shigella; vibrio cholerae; lyme disease; Yersinia pestis; tularemia; and hepatitis (bacterial).

In an embodiment, the invention features a rBCG vector that expresses at least one fungal antigen. Fungal antigens for use with one or more of the rBCG vaccine vectors disclosed herein include, but are not limited to, e.g., candida fungal antigen components; histoplasma fungal antigens, such as heat shock protein 60 (HSP60) and other histoplasma fungal antigen components; cryptococcal fungal antigens, such as capsular polysaccharides and other cryptococcal fungal antigen components; coccidiodes fungal antigens, such as spherule antigens and other coccidiodes fungal antigen components; and tinea fungal antigens, such as trichophytin and other coccidiodes fungal antigen components.

In an embodiment, the invention features a rBCG vector that expresses at least one protozoal or other parasitic antigen. Examples of protozoal and other parasitic antigens for use with one or more of the rBCG vaccine vectors disclosed herein include, but are not limited to, e.g., plasmodium falciparum antigens, such as merozoite surface antigens, sporozoite surface antigens, circumsporozoite antigens, gametocyte/gamete surface antigens, blood-stage antigen pf 155/RESA and other plasmodial antigen components; toxoplasma antigens, such as SAG-1, p30 and other toxoplasmal antigen components; schistosomae antigens, such as glutathione-S-transferase, paramyosin, and other schistosomal antigen components; leishmania major and other leishmaniae antigens, such as gp63, lipophosphoglycan and its associated protein and other leishmanial antigen components; and trypanosoma cruzi antigens, such as the 75-77 kDa antigen, the 56 kDa antigen and other trypanosomal antigen components.

A non-limiting list of pathogens from which an epitope, antigen, or peptide can be derived for inclusion in one or more of the rBCG vaccine vectors described herein is provided in Table 2.

TABLE 2
LIST OF EXAMPLES OF PATHOGENS FROM
WHICH EPITOPES/ANTIGENS/PEPTIDES
CAN BE DERIVED FOR INCLUSION IN
THE rBCG VECTORS OF THE INVENTION

VIRUSES:
Flaviviridae
Yellow Fever virus
Japanese Encephalitis virus
Dengue virus, types 1, 2, 3, & 4
West Nile Virus
Tick Borne Encephalitis virus
Hepatitis C virus (e.g., genotypes 1a, 1b,
2a, 2b, 2c, 3a, 4a, 4b, 4c, and 4d)
Papoviridae:
Papillomavirus
Retroviridae
Human Immunodeficiency virus, type I
Human Immunodeficiency virus, type II
Simian Immunodeficiency virus
Human T lymphotropic virus, types I & II
Hepnaviridae
Hepatitis B virus
Picornaviridae
Hepatitis A virus
Rhinovirus
Poliovirus
Herpesviridae:
Herpes simplex virus, type I
Herpes simplex virus, type II
Cytomegalovirus
Epstein Barr virus
Varicella-Zoster virus
Togaviridae
Alphavirus
Rubella virus
Paramyxoviridae
Respiratory syncytial virus
Parainfluenza virus
Measles virus
Mumps virus
Orthomyxoviridae
Influenza virus
Filoviridae
Marburg virus
Ebola virus
Rotoviridae
Rotavirus
Coronaviridae
Coronavirus
Adenoviridae
Adenovirus
Rhabdoviridae
Rabiesvirus
BACTERIA:
Enterotoxigenic E. coli
Enteropathogenic E. coli
CAMPYLOBACTER JEJUNI
HELICOBACTER PYLORI
SALMONELLA TYPHI
VIBRIO CHOLERAE
Clostridium difficile
Clostridium tetani
Streptococccus pyogenes
Bordetella pertussis
Neisseria meningitides
Neisseria gonorrhoea
Legionella neumophilus
Clamydial spp.
Haemophilus spp.
Shigella spp.
PARASITES:
Plasmodium spp.
Schistosoma spp.
Trypanosoma spp.
Toxoplasma spp.
Cryptosporidia spp.
Pneumocystis spp.
Leishmania spp.

In another embodiment, the invention features a rBCG vector that expresses at least one tumor associated antigen. Examples of tumor associated antigens for use with one or more of the rBCG vaccine vector(s) described herein include tumor proteins, e.g., mutated oncogenes; viral proteins associated with tumors; and tumor mucins and glycolipids. Specific non-limiting examples of tumor antigens include, but are not limited to, CEA, prostate specific antigen (PSA), HER-2/neu, BAGE, GAGE, MAGE 1-4, 6 and 12, MUC-related protein (Mucin) (MUC-1, MUC-2, etc.), GM2 and GD2 gangliosides, ras, myc, tyrosinase, MART (melanoma antigen), MARCO-MART, cyclin B1, cyclin D, Pmel 17(gp100), GnT-V intron V sequence (N-acetylglucoaminyltransferase V intron V sequence), Prostate Ca psm, PRAME (melanoma antigen), β-catcnin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bcl-2, Ki-67, Cyclin B1, gp100, Survivin, and NYESO-1. In another aspect, the antigen is selected from tumor associated antigens that include antigens from leukemias and lymphomas, neurological tumors, such as astrocytomas or glioblastomas, melanoma, breast cancer, lung cancer, head and neck cancer, gastrointestinal tumors, gastric cancer, colon cancer, liver cancer, pancreatic cancer, genitourinary tumors such cervix, uterus, ovarian cancer, vaginal cancer, testicular cancer, prostate cancer or penile cancer, bone tumors, vascular tumors, or cancers of the lip, nasopharynx, pharynx and oral cavity, esophagus, rectum, gall bladder, biliary tree, larynx, lung and bronchus, bladder, kidney, brain and other parts of the nervous system, thyroid, Hodgkin's disease, non-Hodgkin's lymphoma, multiple myeloma and leukemia. In other embodiments, in addition to receiving the rBCG vector, the mammal may also receive chemotherapy, radiotherapy, or surgical therapy to treat the cancer or tumor.

In another embodiment, the invention features a rBCG vector that expresses at least one antigen associated with an autoimmune disease or disorder, an allergy, or graft rejection. For example, an antigen involved in any one or more of the following autoimmune diseases or disorders can be incorporated into one or more of the rBCG vaccine vector(s) of the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens, such as Japanese cedar pollen antigens, ragweed pollen antigens, rye grass pollen antigens, animal derived antigens, such as dust mite antigens and feline antigens, histocompatiblity antigens, and penicillin and other therapeutic drugs. Examples of antigens involved in graft rejection include antigenic components of the graft to be transplanted into the graft recipient, such as heart, lung, liver, pancreas, kidney, and neural graft components. The antigen may be an altered peptide ligand useful in treating an autoimmune disease.

The invention also features a rBCG vector that expresses at least one cytokine. Examples of cytokines for use with one or more of the rBCG vaccine vector(s) described above include, but are not limited to, interleukin-4, IL-5, IL-6, IL-10, IL-12, TGF-β, and TNF-α.

One or more of the rBCG vectors of the invention may also used as an adjuvant or modified to express an adjuvant. Examples of polypeptides that can be expressed as adjuvants in one or more of the rBCG vectors of the invention include, but are not limited to, the A subunit of cholera toxin (i.e. CtxA; Genbank accession no. X00171, AF175708, D30053, D30052,), or parts and/or mutant derivatives thereof (e.g., the A1 domain of the A subunit of Ctx (i.e. CtxA 1; Genbank accession no. K02679)), from any classical Vibrio cholerae (e.g., V. cholerae strain 395, ATCC #39541) or E1 Tor V. cholerae (e.g., V. cholerae strain 2125, ATCC #39050) strain. Alternatively, any bacterial toxin that is a member of the family of bacterial adenosine diphosphate-ribosylating exotoxins (see Krueger and Barbier, Clin. Microbiol. Rev., 8:34; 1995), may be used in place of CtxA, for example the A subunit of heat-labile toxin (referred to herein as EltA) of enterotoxigenic Escherichia coli (Genbank accession #M35581), pertussis toxin S1 subunit (E.g. ptxS1, Genbank accession #AJ007364, AJ007363, AJ006159, AJ006157, etc.); as a further alternative the adjuvant may be one of the adenylate cyclase-hemolysins of Bordetella pertussis (ATCC #8467), Bordetella bronchiseptica (ATCC #7773) or Bordetella parapertussis (ATCC #15237), E.g. the cyaA genes of B. pertussis (Genbank accession no. X14199), B. parapertussis (Genbank accession no. AJ249835) or B. bronchiseptica (Genbank accession no. Z37112).

One or more of the rBCG vectors of the invention may also be used as a vaccine preparation to elicit an immune response against tuberculosis. The vaccine preparations include at least one rBCG strain as described herein, and a pharmacologically suitable carrier. The preparation of such compositions for use as vaccines is well known to those of skill in the art. Typically, such compositions are prepared either as liquid solutions or suspensions, however, solid forms such as tablets, pills, powders and the like are also contemplated. Solid forms suitable for solution in, or suspension in, liquids prior to administration may also be prepared. The preparation may also be emulsified. The active ingredients may be mixed with excipients that are pharmaceutically acceptable and compatible with the active ingredients. Suitable excipients are, for example, water, saline, dextrose, raffinose, glycerol, ethanol and the like, or combinations thereof. In addition, the composition may contain minor amounts of auxiliary substances such as wetting or emulsifying agents, pH buffering agents, and the like. In addition, the composition may contain other adjuvants.

The present invention also provides methods of eliciting an immune response to tuberculosis and methods of vaccinating a mammal against tuberculosis by administering one or more of the rBCG vectors of the invention. By eliciting an immune response, we mean that administration of the vaccine preparation of the present invention causes the synthesis of specific antibodies (at a titer in the range of 1 to 1×10⁶, preferably 1×10³, more preferable in the range of about 1×10³ to about 1×10⁶, and most preferably greater than 1×10⁶) and/or cellular proliferation, as measured, e.g. by ³H thymidine incorporation.

The methods of the present invention involve administering a composition that includes one or more of the rBCG strains of the present invention in a pharmacologically acceptable carrier to a mammal (e.g., a human). The vaccine preparations of the present invention may be administered by any of the many suitable means which are well known to those of skill in the art, including but not limited to injection (e.g., intra-arterial, intravenous, and intrathecal injection), oral, intranasal, intra-pulmonary inoculation, by ingestion of a food product containing the rBCG, etc. In embodiments, the preferred modes of administration include by intra-pulmonary inoculation, by inhalation, and subcutaneous or intramuscular administration.

Although the examples presented above are directed to rBCG vectors (e.g., M. bovis BCG-based vectors), other mycobacterial vectors are envisioned, such as vectors derived from any mycobacterial species, including M. africanum, M. microti, M. leprae, M. smegmatis, M. avium, M. chelonae, M. canetti, M. pinnipedii, M. vacca, M. phlei, M. fortuitum, M. paratuberculosis, M. fortuitum, M. gordonae, M. hiberniae, M. kansasii, M. scrofulaceum, M. intracellulare, M. tuberculosis, M. marinum, M. simiae, M. szulgai, M. ulcerans, and M. xenopi, in which one or more antigens are integrated at the site of one or more of the 15 genes described herein (or their homologs in these other mycobacterial species). Preferably, the mycobacterium is an attenuated strain of a pathogenic mycobacterium. More preferably, the mycobacterium is nonpathogenic (i.e., does not normally cause disease). Examples of nonpathogenic mycobacteria are M. smegmatis, M. phlei, and M. vacca. Most preferably, the mycobacterium is M. smegmatis or M. bovis BCG.

### Pharmaceutical Compositions

Formulations for a BCG vector of the invention can be prepared using standard pharmaceutical formulation chemistries and methodologies that are readily available to the reasonably skilled artisan. For example, rBCG vectors can be combined with one or more pharmaceutically acceptable excipients or vehicles. Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Such compositions may be prepared, packaged, or sold in a form suitable for bolus administration or for continuous administration. Injectable compositions may be prepared, packaged, or sold in unit dosage form, such as in ampoules or in multi-dose containers containing a preservative. Compositions include, but are not limited to, suspensions, solutions, emulsions in oily or aqueous vehicles, pastes, and implantable sustained-release or biodegradable formulations. Such compositions may further comprise one or more additional ingredients including, but not limited to, suspending, stabilizing, or dispersing agents. In one embodiment of a composition for parenteral administration, the active ingredient is provided in dry (for e.g., a powder or granules) form for reconstitution with a suitable vehicle (e.g., sterile pyrogen-free water) prior to parenteral administration of the reconstituted composition. The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Other parentally-administrable compositions that are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Alternatively, the rBCG vectors of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated compositions will include an amount of the rBCG vector of interest that is sufficient to mount an immunological response. An appropriate effective amount can be readily determined by one of skill in the art. Such an amount will fall in a relatively broad range that can be determined through routine trials. The compositions may contain from about 0.1% to about 99.9% of the vector and can be administered directly to the subject or, alternatively, delivered ex vivo, to cells derived from the subject, using methods known to those skilled in the art.

### Immunization Vaccines

The rBCG vector can be administered as a prophylactic or therapeutic vaccine on its own or in combination with other art-known compositions that induce protective responses against pathogens (e.g., viral, bacterial, fungal, or parasitic pathogens), tumors or cancers, allergens, autoimmune disorders, or graft rejection. For example, the rBCG vectors of the present invention can be administered simultaneously, separately, or sequentially with another immunization vaccine, such as a vaccine for, e.g., influenza, malaria, tuberculosis, smallpox, measles, rubella, mumps, or any other vaccines known in the art.

For example, the rBCG vector can be administered as a stand alone vaccine for the treatment of a bacterial, viral, fungal, or parasitic agent, or it can be administered in combination with a secondary bacterial, viral, fungal, or parasite vaccine known in the art for treating a bacterial, viral, fungal, or parasitic agent, respectively. The rBCG and/or secondary vaccine may be directed against a bacterium selected from Pseudomonas aeruginosa, Salmonella typhimurium, Escherichia coli, Klebsiella pneumoniae, Bruscella, Burkholderia mallei, Yersinia pestis, and Bacillus anthracis; a virus selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus; a virus selected from a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus; a virus selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus; a virus selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O'nyong'nyong virus, and the chikungunya virus; a member of the Poxyiridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H₅N₁ avian influenza virus or H₁N₁ swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picornaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types 1 and 2), and human T-lymphotropic virus Types I and II (HTLV-1 and HTLV-2, respectively); or a fungus selected from Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, and Rhizopus arrhizus; or parasite selected from Toxoplasma gondii, Plasmodium falciparum, P. vivax, P. ovale, P. malariae, Trypanosoma spp., and Legionella spp.

Examples of additional secondary vaccines known in the art that can be administered in combination with the rBCG vector compositions of the present invention include AVA (BioThrax) for anthrax; VAR (Varivax) and MMRV (ProQuad) for chickenpox; DTaP (Daptacel, Infanrix, Tripedia), Td (Decavaca, generic), DT (-generic-), Tdap (Boostrix, Adacel), DTaP-IPV (Kinrix), DTaP-HepB-IPV (Pediarix), DTaP-IPV/Hib (Pentacel), and DTaP/Hib (TriHIBit) for Diphtheria; HepA (Havrix, Vaqta) and HepA-HepB (Twinrix) for Hepatitis A; HepB (Engerix-B, Recombivax HB), Hib-HepB (Comvax), DTaP-HepB-IPV (Pediarix), and HepA-HepB (Twinrix) for Hepatitis B; Hib (ActHIB, PedvaxHIB, Hiberix), Hib-HepB (Comvax), DTaP/Hib (TriHIBit), and DTaP-IPV/Hib (Pentacel) for Haemophilus influenzae type b; HPV4 (Gardasil) and HPV2 (Cervarix) for Human Papillomavirus (HPV); TIV (Afluria, Agriflu, FluLaval, Fluarix, Fluvirin, Fluzone) and LAIV (FluMist) for Influenza; JE (Ixiaro and JE-Vax) for Japanese encephalitis (JE); MMR (M-M-R II) and MMRV (ProQuad) for Measles; MCV4 (Menactra), MPSV4 (Menomune), and MODC (Menveo) for Meningitis; MMR (M-M-R II) and MMRV (ProQuad) for Mumps; DTaP (Daptacel, Infanrix, Tripedia), Tdap (Adacel, Boostrix), DTaP-IPV (Kinrix), DTaP-HepB-IPV (Pediarix), DTaP-IPV/Hib (Pentacel), and DTaP/Hib (TriHBit) for Pertussis; PCV7 (Prevnar), PCV13 (Prevnar13), and PPSV23 (Pneumovax 23) for Bacterial Pneumonia; Polio (Ipol), DTaP-IPV (Kinrix), DTaP-HepB-IPV (Pediarix), and DTaP-IPV/Hib (Pentacel) for Polio; Rabies (Imovax Rabies and RabAvert); RV1 (Rotarix) and RV5 (RotaTeq) for Rotavirus; MMR (M-M-R II) and MMRV (ProQuad) for Rubella; ZOS (Zostavax) for Shingles; Vaccinia (ACAM2000, Dryvax) for Smallpox and Monkeypox; DTaP (Daptacel, Infanrix, Tripedia), Td (Decavac, generic), DT (-generic-), TT (-generic-), Tdap (Boostrix, Adacel), DTaP-IPV (Kinrix), DTaP-HepB-IPV (Pediarix), DTaP-IPV/Hib (Pentacel), and DTaP/Hib (TriHIBit) for Tetanus; BCG (TICE BCG, Mycobax) for Tuberculosis (TB); Typhoid Oral (Vivotif) and Typhoid Polysaccharide (Typhim Vi) for Typhoid; and YF (YF-Vax) for Yellow Fever.

Immunization vaccines of the present invention include an effective amount of a mycobacterial vector described herein (e.g., a rBCG vector) typically dissolved or dispersed in a pharmaceutically acceptable carrier. The phrases “pharmaceutical or pharmacologically acceptable” refers to molecular entities and compositions that, on their own, do not produce an adverse, allergic or other untoward reaction when administered to an animal, such as, for example, a human, as appropriate, and include, e.g., any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. The preparation of a pharmaceutical composition that contains a mycobacterial vector will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards. A specific example of a pharmacologically acceptable carrier as described herein is the sterile saline solution (0.9% NaCl) with or without placebo (RPMI 1640 with 8% human serum albumin and 20% (by volume) RIMSO™-50).

The rBCG vector compositions of the invention, whether administered as an immunization vaccine or not, may be administered by direct intradermal injection or intra-pulmonary inoculation. Other methods of administration are contemplated may be used in some cases. It will be understood by of skill in the art that any form of systemic administration will preferable involve a dosage that may include about 1×10³ to about 1×10¹², e.g., about 1×10³, about 1×10⁴, about 1×10⁵, or about 1×10⁶ CFU of a recombinant mycobacterial vector of the invention (e.g., a rBCG vector of the invention). Thus, the mycobacterial vector of the present invention (e.g., a rBCG vector of the invention) can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), by intra-pulmonary inoculation, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a mycobacterial vector (e.g,. a rBCG vector) of the present invention administered to an animal patient (e.g., a human) can be determined by physical and physiological factors such as PPD antigen reactivity, assessment of the transgene-product specific T cell response, general immune status, body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 5 mg/kg/body weight to about 100 mg/kg/body weight, about 5 microgram/kg/body weight to about 500 milligram/kg/body weight, etc., can be administered, based on the numbers described above.

Sterile injectable solutions are prepared by incorporating the active components (e.g., a rBCG vector of the invention) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

In particular embodiments, prolonged absorption of an injectable composition can be brought about by the use in the compositions of agents delaying absorption, such as, for example, aluminum monostearate, gelatin or combinations thereof.

### Delivery Methods

Once formulated, the rBCG vector compositions of the invention can be delivered to a mammalian subject (e.g., a human or other mammal described herein) in vivo using a variety of known routes and techniques. For example, a composition can be provided as an injectable solution, suspension or emulsion and administered via parenteral, subcutaneous, epidermal, intradermal, intramuscular, intraarterial, intraperitoneal, and intravenous injection and by intra-pulmonary inoculation using a conventional needle and syringe, a liquid jet injection system, or other methods known in the art. Compositions can also be administered topically to skin or mucosal tissue, such as nasally, intratracheally, intestinal, rectally or vaginally, or provided as a finely divided spray suitable for respiratory or pulmonary administration (e.g., for intra-pulmonary inoculation). Other modes of administration include oral administration, suppositories, and active or passive transdermal delivery techniques. Particularly in relation to the present invention, compositions may be administered directly to the gastrointestinal tract.

Alternatively, the rBCG vector compositions can be administered ex vivo, for example, by delivery and reimplantation of transformed cells into a mammalian subject (e.g., a human or other mammal described herein).

### Delivery Regimes

The rBCG vector compositions of the present invention are administered to a mammalian subject (e.g., a human or other mammal described herein) in an amount that is compatible with the dosage formulation and that will be prophylactically and/or therapeutically effective. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed.

As used herein, the term “prophylactically or therapeutically effective dose” means a dose in an amount sufficient to elicit an immune response to one or more epitopes of a polypeptide incorporated into a rBCG vector of the invention and/or to alleviate, reduce, cure or at least partially arrest symptoms and/or complications from a disease or infection for which the rBCG vector is administered.

Prophylaxis or therapy can be accomplished by a single direct administration at a single time point or by multiple administrations, optionally at multiple time points. Administration can also be delivered to a single or to multiple sites. Those skilled in the art can adjust the dosage and concentration to suit the particular route of delivery. In one embodiment, a single dose is administered on a single occasion. In an alternative embodiment, a number of doses are administered to a subject on the same occasion but, for example, at different sites. In a further embodiment, multiple doses are administered on multiple occasions. Such multiple doses may be administered in batches, i.e. with multiple administrations at different sites on the same occasion, or may be administered individually, with one administration on each of multiple occasions (optionally at multiple sites). Any combination of such administration regimes may be used.

In one embodiment, different compositions of the invention may be administered at different sites or on different occasions as part of the same treatment regime. It is known that improved immune responses may be generated to an antigen by varying the vectors used to deliver the antigen. There is evidence that in some instances antibody and/or cellular immune responses may be improved by using two different vectors administered sequentially as a “prime” and a “boost.”

For example, a rBCG vector of the invention that expresses one or more of the antigens described herein may be administered as a “prime” in one composition, and the antigen may subsequently be administered as a “boost” in a different composition. The two vaccine compositions may differ in the choice of vector comprising the antigen. For example, the “boost” vector may be selected from a plasmid vector (e.g., a DNA vector), a poxvirus vector, an adenovirus vector, or other vector known in the art and may be administered sequentially after the rBCG vector. In the most common cases, the two different vectors would carry a common transgenic antigen.

In such a prime-boost protocol, one or more administrations of the prime and/or the boost may be performed. For example, the prime and/or boost step may be achieved using a single administration or using two or more administrations at different sites and/or on different occasions. In one embodiment, two administrations on different occasions are given for the prime step and a single administration on a later occasion is given for the boost step.

Different administrations may be performed on the same occasion, on the same day, one, two, three, four, five or six days apart, one, two, three, four or more weeks apart. Preferably, administrations are 1 to 5 weeks apart, more preferably 2 to 4 weeks apart, such as 2 weeks, 3 weeks or 4 weeks apart. The schedule and timing of such multiple administrations can be optimised for a particular composition or compositions by one of skill in the art by routine trials.

In certain embodiments of the invention, it is preferable that a particular dosage of the mycobacterial vector (e.g., a rBCG vector having one or more of mutations that ablate expression of, e.g., one or more genes selected from BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, BCG_{—}3808c, or the equivalent homologs thereof in a mycobacterial species other than M. bovis BCG, or the operon that includes one or more of these gene) be administered to a mammalian subject (e.g., a human or other mammal described herein). Thus, in certain embodiments of the invention, there is provided a containment means that includes 1 to 5 unit doses of a mycobacterial vector (e.g., the rBCG vector), in which each unit dose includes about 1×10³ to about 1×10¹² CFU of the mycobacterial vector (e.g., the rBCG vector). In some further embodiments of the invention, there is provided a composition comprising 1 to 5 unit doses of about 1×10⁵ to about 5×10⁶ CFU of the mycobacterial vector (e.g., the rBCG vector) in a suitable containment means. In very specific embodiments of the invention, there is also provided a composition that includes: 1 unit dose of about 3×10⁶ CFU of the mycobacterial vector (e.g., the rBCG vector); 2 unit doses of about 1.5×10⁶ CFU of the mycobacterial vector (e.g., the rBCG vector), or 4 unit doses of about 7.5×10⁵ CFU of the mycobacterial vector (e.g., the rBCG vector) in a suitable containment means. A container according to the invention in certain instances, may be a vial, an ampoule, a syringe or a tube. In some cases, the mycobacterial vector (e.g., the rBCG vector) may be lyophilized and formulated for resuspension prior to administration. However, in other cases, the mycobacterial vector (e.g., the rBCG vector) is suspended in a volume of a pharmaceutically acceptable liquid. In some of the most preferred embodiments there is provided a container that includes a single unit dose of the mycobacterial vector (e.g., the rBCG vector of the present invention) suspended in pharmaceutically acceptable carrier wherein the unit dose includes about 1×10 to about 1×10⁷ CFU of the mycobacterial vector (e.g., the rBCG vector). In some very specific embodiments the liquid comprising the suspended mycobacterial vector (e.g., the rBCG vector) is provided in a volume of between about 0.1 ml and 10 mls, or about 0.5 ml and 2 mls. In very specific case the suspended mycobacterial vector (e.g., the rBCG vector) is provided in a volume of about 1 ml. It will further be understood that in certain instances a composition comprising the mycobacterial vector (e.g., the rBCG vector) in a containment means is frozen (i.e. maintained at less than about 0° C.). The foregoing compositions provide ideal units for immunotherapeutic applications described herein.

In some further embodiments of the invention, methods of the invention involve the administration of 2, 3, 4, 5, 6, 7, 8, 9, 10 or more doses of the mycobacterial vector (e.g., the rBCG vector) separated by a period of one day or more. In certain preferred embodiments such separate doses will be separated by several days, one week, two weeks, one month or more. Such a separation of the doses is preferable due to superficial abscess formation that typically accompanies such therapy. For example, methods according to the invention may comprise administering 1 to 5 doses of the mycobacterial vector (e.g., the rBCG vector) over a period of three weeks or more. In yet further embodiments, methods of the invention comprise administering 1 to 5, 1 to 4, 1 to 3, 1 to 2 or 2 doses of the mycobacterial vector (e.g., the rBCG vector) over a period of about three weeks. Each dose administered may be the same or different dosage relative to a previous or subsequent dose administration. In certain cases, it may be preferred that a mycobacterial vector-based (e.g., the rBCG vector-based) immunotherapy be administered a minimal number of times, for example, in less than 10, 9, 8, 7, 6, 5, 4, 3 or fewer separate dosage administrations. In some cases the mycobacterial vector (e.g., the rBCG vector) composition is administered twice.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All publications and patent applications mentioned in this specification are herein incorporated by reference to the same extent as if each independent publication or patent application was specifically and individually indicated to be incorporated by reference in their entirety.

### BIBLIOGRAPHY

1. Bennink J R, Yewdell J W, Smith G L, Moller C, Moss B. Recombinant vaccinia virus primes and stimulates influenza haemagglutinin-specific cytotoxic T cells. Nature 1984; 311 (5986):578-9.
2. BCG vaccine. WHO position paper. Wkly Epidemiol Rec 2004; 79(4):27-38.
3. Levine M M, Sztein M B. Vaccine development strategies for improving immunization: the role of modern immunology. Nat Immunol 2004; 5(5):460-4.
4. Potential use of live viral and bacterial vectors for vaccines. WHO meeting, Geneva, 19-22 June, 1989. Vaccine 1990; 8(5):425-37.
5. Yu J S, Peacock J W, Vanleeuwen S, et al. Generation of mucosal anti-human immunodeficiency virus type 1 T-cell responses by recombinant Mycobacterium smegmatis. Clin Vaccine Immunol 2006; 13(11):1204-11.
6. Krieg A M. CpG motifs in bacterial DNA and their immune effects. Annu Rev Immunol 2002; 20:709-60.
7. Yu J S, Peacock J W, Jacobs W R, Jr., Frothingham R, Letvin N L, Liao H X, Haynes B F. Recombinant Mycobacterium bovis bacillus Calmette-Guerin elicits human immunodeficiency virus type 1 envelope-specific T lymphocytes at mucosal sites. Clin Vaccine Immunol 2007; 14(7):886-93.
8. Lim E M, Lagranderie M, Le Grand R, Rauzier J, Gheorghiu M, Gicquel B, Winter N. Recombinant Mycobacterium bovis BCG producing the N-terminal half of SIVmac251 Env antigen induces neutralizing antibodies and cytotoxic T lymphocyte responses in mice and guinea pigs. AIDS Res Hum Retroviruses 1997; 13(18):1573-81.
9. Cayabyab M J, Korioth-Schmitz B, Sun Y, et al. Recombinant Mycobacterium bovis BCG prime-recombinant adenovirus boost vaccination in rhesus monkeys elicits robust polyfunctional simian immunodeficiency virus-specific T-cell responses. J Virol 2009; 83(11):5505-13.
10. Edelman R, Palmer K, Russ K G, et al. Safety and immunogenicity of recombinant Bacille Calmette-Guerin (rBCG) expressing Borrelia burgdorferi outer surface protein A (OspA) lipoprotein in adult volunteers: a candidate Lyme disease vaccine. Vaccine 1999; 17(7-8):904-14.
11. Ami Y, Izumi Y, Matsuo K, et al. Priming-boosting vaccination with recombinant Mycobacterium bovis bacillus Calmette-Guerin and a nonreplicating vaccinia virus recombinant leads to long-lasting and effective immunity. J Virol 2005; 79(20):12871-9.
12. Yasutomi Y, Koenig S, Haun S S, et al. Immunization with recombinant BCG-SIV elicits SIV-specific cytotoxic T lymphocytes in rhesus monkeys. J Immunol 1993; 150(7):3101-7.
13. Kaufmann S H. Tuberculosis: back on the immunologists' agenda. Immunity 2006; 24(4):351-7.
14. Jang J, Becq J, Gicquel B, Deschavanne P, Neyrolles O. Horizontally acquired genomic islands in the tubercle bacilli. Trends Microbiol 2008; 16(7):303-8.
15. Garnier T, Eiglmeier K, Camus J C, et al. The complete genome sequence of Mycobacterium bovis. Proc Natl Acad Sci USA 2003; 100(13):7877-82.
16. Smith N H, Gordon S V, de la Rua-Domenech R, Clifton-Hadley R S, Hewinson R G. Bottlenecks and broomsticks: the molecular evolution of Mycobacterium bovis. Nat Rev Microbiol 2006; 4(9):670-81.
17. Perna N T, Plunkett G, 3rd, Burland V, et al. Genome sequence of enterohaemorrhagic Escherichia coli O157:H7. Nature 2001; 409(6819):529-33.
18. Brosch R, Gordon S V, Garnier T, et al. Genome plasticity of BCG and impact on vaccine efficacy. Proc Natl Acad Sci USA 2007; 104(13):5596-601.
19. Hsu T, Hingley-Wilson S M, Chen B, et al. The primary mechanism of attenuation of bacillus Calmette-Guerin is a loss of secreted lytic function required for invasion of lung interstitial tissue. Proc Natl Acad Sci USA 2003; 100(21):12420-5.
20. Lewis K N, Liao R, Guinn K M, Hickey M J, Smith S, Behr M A, Sherman D R. Deletion of RD1 from Mycobacterium tuberculosis mimics bacille Calmette-Guerin attenuation. J Infect Dis 2003; 187(1):117-23.
21. Fortune S M, Jaeger A, Sarracino D A, Chase M R, Sassetti C M, Sherman D R, Bloom B R, Rubin E J. Mutually dependent secretion of proteins required for mycobacterial virulence. Proc Natl Acad Sci USA 2005; 102(30):10676-81.
22. Behr M A. BCG—different strains, different vaccines? Lancet Infect Dis 2002; 2(2):86-92.
23. Kozak R A, Alexander D C, Liao R, Sherman D R, Behr M A. Region of difference 2 contributes to virulence of Mycobacterium tuberculosis. Infect Immun; 79(1):59-66.
24. Behr M A, Schroeder B G, Brinkman J N, Slayden R A, Barry C E, 3rd. A point mutation in the mma3 gene is responsible for impaired methoxymycolic acid production in Mycobacterium bovis BCG strains obtained after 1927. J Bacteriol 2000; 182(12):3394-9.
25. Liu J, Tran V, Leung A S, Alexander D C, Zhu B. BCG vaccines: their mechanisms of attenuation and impact on safety and protective efficacy. Hum Vaccin 2009; 5(2):70-8.
26. Belley A, Alexander D, Di Pietrantonio T, et al. Impact of methoxymycolic acid production by Mycobacterium bovis BCG vaccines. Infect Immun 2004; 72(5):2803-9.
27. Ritz N, Hanekom W A, Robins-Browne R, Britton W J, Curtis N. Influence of BCG vaccine strain on the immune response and protection against tuberculosis. FEMS Microbiol Rev 2008; 32(5):821-41.
28. Bastos R G, Borsuk S, Seixas F K, Dellagostin O A. Recombinant Mycobacterium bovis BCG. Vaccine 2009; 27(47):6495-503.
29. Aggarwal A, Dutta A K. Timing and dose of BCG vaccination in infants as assessed by postvaccination tuberculin sensitivity. Indian Pediatr 1995; 32(6):635-9.
30. Manufactured product: BCG Vaccine.
31. BCG Vaccine.
32. Antony V B, Sahn S A, Antony A C, Repine J E. Bacillus Calmette-Guerin-stimulated neutrophils release chemotaxins for monocytes in rabbit pleural spaces and in vitro. J Clin Invest 1985; 76(4):1514-21.
33. Suttmann H, Lehan N, Bohle A, Brandau S. Stimulation of neutrophil granulocytes with Mycobacterium bovis bacillus Calmette-Guerin induces changes in phenotype and gene expression and inhibits spontaneous apoptosis. Infect Immun 2003; 71(8):4647-56.
34. Ramos-Kichik V, Mondragon-Flores R, Mondragon-Castelan M, et al. Neutrophil extracellular traps are induced by Mycobacterium tuberculosis. Tuberculosis (Edinb) 2009; 89(1):29-37.
35. Abadie V, Badell E, Douillard P, et al. Neutrophils rapidly migrate via lymphatics after Mycobacterium bovis BCG intradermal vaccination and shuttle live bacilli to the draining lymph nodes. Blood 2005; 106(5):1843-50.
36. Geissmann F, Jung S, Littman D R. Blood monocytes consist of two principal subsets with distinct migratory properties. Immunity 2003; 19(1):71-82.
37. Schorey J S, Carroll M C, Brown E J. A macrophage invasion mechanism of pathogenic mycobacteria. Science 1997; 277(5329):1091-3.
38. Schlesinger L S, Hull S R, Kaufman T M. Binding of the terminal mannosyl units of lipoarabinomannan from a virulent strain of Mycobacterium tuberculosis to human macrophages. Immunol 1994; 152(8):4070-9.
39. Schlesinger L S, Kaufman T M, Iyer S, Hull S R, Marchiando L K. Differences in mannose receptor-mediated uptake of lipoarabinomannan from virulent and attenuated strains of Mycobacterium tuberculosis by human macrophages. J Immunol 1996; 157(10):4568-75.
40. Hawkes M, Li X, Crockett M, et al. CD36 deficiency attenuates experimental mycobacterial infection. BMC Infect Dis; 10:299.
41. Ernst J D. Macrophage receptors for Mycobacterium tuberculosis. Infect Immun 1998; 66(4):1277-81.
42. Stenger S, Modlin R L. Control of Mycobacterium tuberculosis through mammalian Toll-like receptors. Curr Opin Immunol 2002; 14(4):452-7.
43. Connelly M A, Moulton R A, Smith A K, Lindsey D R, Sinha M, Wetsel R A, Jagannath C. Mycobacteria-primed macrophages and dendritic cells induce an up-regulation of complement C5a anaphylatoxin receptor (CD88) in CD3+ murine T cells. J Leukoc Biol 2007; 81(1):212-20.
44. Yang C S, Shin D M, Kim K H, Lee Z W, Lee C H, Park S G, Bae Y S, Jo E K. NADPH oxidase 2 interaction with TLR2 is required for efficient innate immune responses to mycobacteria via cathelicidin expression. J Immunol 2009; 182(6):3696-705.
45. Miller J L, Velmurugan K, Cowan M J, Briken V. The type I NADH dehydrogenase of Mycobacterium tuberculosis counters phagosomal NOX2 activity to inhibit TNF-alpha-mediated host cell apoptosis. PLoS Pathog; 6(4):e1000864.
46. de Chastellier C, Forquet F, Gordon A, Thilo L. Mycobacterium requires an all-around closely apposing phagosome membrane to maintain the maturation block and this apposition is re-established when it rescues itself from phagolysosomes. Cell Microbiol 2009; 11(8):1190-207.
47. Astarie-Dequeker C, Le Guyader L, Malaga W, Seaphanh F K, Chalut C, Lopez A, Guilhot C. Phthiocerol dimycocerosates of M. tuberculosis participate in macrophage invasion by inducing changes in the organization of plasma membrane lipids. PLoS Pathog 2009; 5(2):e1000289.
48. Seto S, Matsumoto S, Tsujimura K, Koide Y. Differential recruitment of CD63 and Rab7-interacting-lysosomal-protein to phagosomes containing Mycobacterium tuberculosis in macrophages. Microbiol. Immunol; 54(3):170-4.
49. Cardoso C M, Jordao L, Vieira O V. Rab10 regulates phagosome maturation and its overexpression rescues Mycobacterium-containing phagosomes maturation. Traffic; 11(2):221-35.
50. Sun J, Deghmane A E, Soualhine H, Hong T, Bucci C, Solodkin A, Hmama Z. Mycobacterium bovis BCG disrupts the interaction of Rab7 with RILP contributing to inhibition of phagosome maturation. J Leukoc Biol 2007; 82(6):1437-45.
51. van der Wel N, Hava D, Houben D, Fluitsma D, van Zon M, Pierson J, Brenner M, Peters P J. M. tuberculosis and M. leprae translocate from the phagolysosome to the cytosol in myeloid cells. Cell 2007; 129(7):1287-98.
52. Caruso A M, Serbina N, Klein E, Triebold K, Bloom B R, Flynn J L. Mice deficient in CD4 T cells have only transiently diminished levels of IFN-gamma, yet succumb to tuberculosis. J Immunol 1999; 162(9):5407-16.
53. Flynn J L, Goldstein M M, Triebold K J, Bloom B R. Major histocompatibility complex class I-restricted T cells are necessary for protection against M. tuberculosis in mice. Infect Agents Dis 1993; 2(4):259-62.
54. Woodworth J S, Wu Y, Behar S M. Mycobacterium tuberculosis-specific CD8+ T cells require perforin to kill target cells and provide protection in vivo. J Immunol 2008; 181(12):8595-603.
55. MacMicking J D, Taylor G A, McKinney J D. Immune control of tuberculosis by IFN-gamma-inducible LRG-47. Science 2003; 302(5645):654-9.
56. Ehrt S, Schnappinger D. Mycobacterial survival strategies in the phagosome: defence against host stresses. Cell Microbiol 2009; 11(8):1170-8.
57. Kisich K O, Higgins M, Diamond G, Heifets L. Tumor necrosis factor alpha stimulates killing of Mycobacterium tuberculosis by human neutrophils. Infect Immun 2002; 70(8):4591-9.
58. Mittrucker H W, Steinhoff U, Kohler A, Krause M, Lazar D, Mex P, Miekley D, Kaufmann S H. Poor correlation between BCG vaccination-induced T cell responses and protection against tuberculosis. Proc Natl Acad Sci USA 2007; 104(30):12434-9.
59. Chen C Y, Huang D, Wang R C, et al. A critical role for CD8 T cells in a nonhuman primate model of tuberculosis. PLoS Pathog 2009; 5(4):e1000392.
60. Serbina N V, Flynn J L. CD8(+) T cells participate in the memory immune response to Mycobacterium tuberculosis. Infect Immun 2001; 69(7):4320-8.
61. Serbina N V, Lazarevic V, Flynn J L. CD4(+) T cells are required for the development of cytotoxic CD8(+) T cells during Mycobacterium tuberculosis infection. J Immunol 2001; 167(12):6991-7000.
62. Mogues T, Goodrich M E, Ryan L, LaCourse R, North R J. The relative importance of T cell subsets in immunity and immunopathology of airborne Mycobacterium tuberculosis infection in mice. J Exp Med 2001; 193(3):271-80.
63. Chow A, Toomre D, Garrett W, Mellman I. Dendritic cell maturation triggers retrograde MHC class II transport from lysosomes to the plasma membrane. Nature 2002; 418(6901):988-94.
64. Woodworth J S, Fortune S M, Behar S M. Bacterial protein secretion is required for priming of CD8+ T cells specific for the Mycobacterium tuberculosis antigen CFP10. Infect Immun 2008; 76(9):4199-205.
65. Lewinsohn D M, Grotzke J E, Heinzel A S, Zhu L, Ovendale P J, Johnson M, Alderson M R. Secreted proteins from Mycobacterium tuberculosis gain access to the cytosolic MHC class-I antigen-processing pathway. J Immunol 2006; 177(1):437-42.
66. Billeskov R, Vingsbo-Lundberg C, Andersen P, Dietrich J. Induction of CD8 T cells against a novel epitope in TB10.4: correlation with mycobacterial virulence and the presence of a functional region of difference-1. J Immunol 2007; 179(6):3973-81.
67. Weerdenburg E M, Peters P J, van der Wel N N. How do mycobacteria activate CD8+ T cells? Trends Microbiol; 18(1):1-10.
68. Majlessi L, Combaluzier B, Albrecht I, Garcia J E, Nouze C, Pieters J, Leclerc C. Inhibition of phagosome maturation by mycobacteria does not interfere with presentation of mycobacterial antigens by MHC molecules. J Immunol 2007; 179(3):1825-33.
69. Rock K L, Gamble S, Rothstein L. Presentation of exogenous antigen with class I major histocompatibility complex molecules. Science 1990; 249(4971):918-21.
70. Ackerman A L, Kyritsis C, Tampe R, Cresswell P. Access of soluble antigens to the endoplasmic reticulum can explain cross-presentation by dendritic cells. Nat Immunol 2005; 6(1):107-13.
71. Ackerman A L, Kyritsis C, Tampe R, Cresswell P. Early phagosomes in dendritic cells form a cellular compartment sufficient for cross presentation of exogenous antigens. Proc Natl Acad Sci USA 2003; 100(22):12889-94.
72. Denzer K, Kleijmeer M J, Heijnen H F, Stoorvogel W, Geuze H J. Exosome: from internal vesicle of the multivesicular body to intercellular signaling device. J Cell Sci 2000; 113 Pt 19:3365-74.
73. Bhatnagar S, Schorey J S. Exosomes released from infected macrophages contain Mycobacterium avium glycopeptidolipids and are proinflammatory. J Biol Chem 2007; 282(35):25779-89.
74. Girl P K, Schorey J S. Exosomes derived from M. Bovis BCG infected macrophages activate antigen-specific CD4+ and CD8+ T cells in vitro and in vivo. PLoS One 2008; 3(6):e2461.
75. Schaible U E, Winau F, Sieling P A, et al. Apoptosis facilitates antigen presentation to T lymphocytes through MHC-I and CD1 in tuberculosis. Nat Med 2003; 9(8):1039-46.
76. Chen M, Gan H, Remold H G. A mechanism of virulence: virulent Mycobacterium tuberculosis strain H37Rv, but not attenuated H37Ra, causes significant mitochondrial inner membrane disruption in macrophages leading to necrosis. J Immunol 2006; 176(6):3707-16.
77. Behar S M, Divangahi M, Remold H G. Evasion of innate immunity by Mycobacterium tuberculosis: is death an exit strategy? Nat Rev Microbiol; 8(9):668-74.
78. Divangahi M, Desjardins D, Nunes-Alves C, Remold H G, Behar S M. Eicosanoid pathways regulate adaptive immunity to Mycobacterium tuberculosis. Nat Immunol; 11(8):751-8.
79. Behar S M, Martin C J, Booty M G, Nishimura T, Zhao X, Gan H X, Divangahi M, Remold H G. Apoptosis is an innate defense function of macrophages against Mycobacterium tuberculosis. Mucosal Immunol.
80. Keane J, Remold H G, Komfeld H. Virulent Mycobacterium tuberculosis strains evade apoptosis of infected alveolar macrophages. J Immunol 2000; 164(4):2016-20.
81. Woodworth J S, Shin D, Volman M, Nunes-Alves C, Fortune S M, Behar S M. Mycobacterium tuberculosis directs immunofocusing of CD8+ T cell responses despite vaccination. J Immunol 2011; 186(3):1627-37.
82. Moody D B, Young D C, Cheng T Y, et al. T cell activation by lipopeptide antigens. Science 2004; 303(5657):527-31.
83. Van Rhijn I, Young D C, De Jong A, et al. CD1c bypasses lysosomes to present a lipopeptide antigen with 12 amino acids. J Exp Med 2009; 206(6):1409-22.
84. Van Rhijn I, Zajonc D M, Wilson I A, Moody D B. T-cell activation by lipopeptide antigens. Curr Opin Immunol 2005; 17(3):222-9.
85. Hava D L, van der Wel N, Cohen N, et al. Evasion of peptide, but not lipid antigen presentation, through pathogen-induced dendritic cell maturation. Proc Natl Acad Sci USA 2008; 105(32):11281-6.
86. Parekh V V, Wilson M T, Olivares-Villagomez D, Singh A K, Wu L, Wang C R, Joyce S, Van Kaer L. Glycolipid antigen induces long-term natural killer T cell anergy in mice. J Clin Invest 2005; 115(9):2572-83.
87. Felio K, Nguyen H, Dascher C C, et al. CD1-restricted adaptive immune responses to Mycobacteria in human group 1 CD1 transgenic mice. J Exp Med 2009; 206(11):2497-509.
88. Abebe F, Bjune G. The protective role of antibody responses during Mycobacterium tuberculosis infection. Clin Exp Immunol 2009; 157(2):235-43.
89. Vordermeier H M, Venkataprasad N, Harris D P, Ivanyi J. Increase of tuberculous infection in the organs of B cell-deficient mice. Clin Exp Immunol 1996; 106(2):312-6.
90. Maglione P J, Chan J. How B cells shape the immune response against Mycobacterium tuberculosis. Eur J Immunol 2009; 39(3):676-86.
91. Maglione P J, Xu J, Chan J. B cells moderate inflammatory progression and enhance bacterial containment upon pulmonary challenge with Mycobacterium tuberculosis. J Immunol 2007; 178(11):7222-34.
92. Glatman-Freedman A, Casadevall A. Serum therapy for tuberculosis revisited: reappraisal of the role of antibody-mediated immunity against Mycobacterium tuberculosis. Clin Microbiol Rev 1998; 11(3):514-32.
93. Lopez Y, Yero D, Falero-Diaz G, et al. Induction of a protective response with an IgA monoclonal antibody against Mycobacterium tuberculosis 16 kDa protein in a model of progressive pulmonary infection. Int J Med Microbiol 2009; 299(6):447-52.
94. Hamasur B, Haile M, Pawlowski A, et al. Mycobacterium tuberculosis arabinomannan-protein conjugates protect against tuberculosis. Vaccine 2003; 21(25-26):4081-93.
95. Balu S, Reljic R, Lewis M J, et al. A Novel Human IgA Monoclonal Antibody Protects against Tuberculosis. J Immunol; 186(5):3113-9.
96. Choucroun N. Precipitin test for carbohydrate antibodies in human tuberculosis. Am Rev Tubere 1949; 59(6):710-2.
97. Seibert F B, Miller E E, Buseman U, Seibert M V, Soto-Figueroa E, Fry L. The significance of antibodies to tuberculoprotein and polysaccharide in resistance to tuberculosis; interference with antibodies by these antigens. Am Rev Tuberc 1956; 73(4):547-62.
98. Costello A M, Kumar A, Narayan V, et al. Does antibody to mycobacterial antigens, including lipoarabinomannan, limit dissemination in childhood tuberculosis? Trans R Soc Trop Med Hyg 1992; 86(6):686-92.
99. de Valliere S, Abate G, Blazevic A, Heuertz R M, Hoft D F. Enhancement of innate and cell-mediated immunity by antimycobacterial antibodies. Infect Immun 2005; 73(10):6711-20.
100. Cocito C G. Properties of the mycobacterial antigen complex A60 and its applications to the diagnosis and prognosis of tuberculosis. Chest 1991; 100(6): 1687-93.
101. Wilkinson R J, Wilkinson K A, De Smet K A, Haslov K, Pasvol G, Singh M, Svarcova I, Ivanyi J. Human T- and B-cell reactivity to the 16 kDa alpha-crystallin protein of Mycobacterium tuberculosis. Scand J Immunol 1998; 48(4):403-9.
102. Fujita Y, Doi T, Sato K, Yano I. Diverse humoral immune responses and changes in IgG antibody levels against mycobacterial lipid antigens in active tuberculosis. Microbiology 2005; 151(Pt 6):2065-74.
103. Hendrickson R C, Douglass J F, Reynolds L D, McNeill P D, Carter D, Reed S G, Houghton R L. Mass spectrometric identification of mtb81, a novel serological marker for tuberculosis. J Clin Microbiol 2000; 38(6):2354-61.
104. Harboe M, Wiker H G. The 38-kDa protein of Mycobacterium tuberculosis: a review. J Infect Dis 1992; 166(4):874-84.
105. Wilkinson R J, Haslov K, Rappuoli R, et al. Evaluation of the recombinant 38-kilodalton antigen of Mycobacterium tuberculosis as a potential immunodiagnostic reagent. J Clin Microbiol 1997; 35(3):553-7.
106. Pottumarthy S, Wells V C, Morris A J. A comparison of seven tests for serological diagnosis of tuberculosis. J Clin Microbiol 2000; 38(6):2227-31.
107. Demkow U, Ziolkowski J, Filewska M, et al. Diagnostic value of different serological tests for tuberculosis in Poland. J Physiol Pharmacol 2004; 55 Suppl 3:57-66.
108. Demkow U, Ziolkowski J, Bialas-Chromiec B, Filewska M, Zielonka T, Wasik M, Rowinska-Zakrzewska E. Humoral immune response against mycobacterial antigens in children with tuberculosis. J Physiol Pharmacol 2006; 57 Suppl 4:63-73.
109. Verma R K, Jain A. Antibodies to mycobacterial antigens for diagnosis of tuberculosis. FEMS Immunol Med Microbiol 2007; 51(3):453-61.
110. Gutierrez M C, Brisse S, Brosch R, Fabre M, Omais B, Marmiesse M, Supply P, Vincent V. Ancient origin and gene mosaicism of the progenitor of Mycobacterium tuberculosis. PLoS Pathog 2005; 1(1):e5.
111. Becq J, Gutierrez M C, Rosas-Magallanes V, Rauzier J, Gicquel B, Neyrolles O, Deschavanne P. Contribution of horizontally acquired genomic islands to the evolution of the tubercle bacilli. Mol Biol Evol 2007; 24(8):1861-71.
112. Zink A R, Sola C, Reischl U, Grabner W, Rastogi N, Wolf H, Nerlich A G. Characterization of Mycobacterium tuberculosis complex DNAs from Egyptian mummies by spoligotyping. J Clin Microbiol 2003; 41(1):359-67.
113. Hershkovitz I, Donoghue H D, Minnikin D E, et al. Detection and molecular characterization of 9,000-year-old Mycobacterium tuberculosis from a Neolithic settlement in the Eastern Mediterranean. PLoS One 2008; 3(10):e3426.
114. Kuijl C, Neefjes J. New insight into the everlasting host-pathogen arms race. Nat Immunol 2009; 10(8):808-9.
115. Gercken J, Pryjma J, Ernst M, Flad H D. Defective antigen presentation by Mycobacterium tuberculosis-infected monocytes. Infect Immun 1994; 62(8):3472-8.
116. Benson S A, Ernst J D. TLR2-dependent inhibition of macrophage responses to IFN-gamma is mediated by distinct, gene-specific mechanisms. PLoS One 2009; 4(7):e6329.
117. van den Elsen P J, Holling T M, Kuipers H F, van der Stoep N. Transcriptional regulation of antigen presentation. Curr Opin Immunol 2004; 16(1):67-75.
118. Wu X, Kong X, Luchsinger L, Smith B D, Xu Y. Regulating the activity of class II transactivator by posttranslational modifications: exploring the possibilities. Mol Cell Biol 2009; 29(21):5639-44.
119. Pennini M E, Liu Y, Yang J, Croniger C M, Boom W H, Harding C V. CCAAT/enhancer-binding protein beta and delta binding to CIITA promoters is associated with the inhibition of CIITA expression in response to Mycobacterium tuberculosis 19-kDa lipoprotein. J Immunol 2007; 179(10):6910-8.
120. Pai R K, Convery M, Hamilton T A, Boom W H, Harding C V. Inhibition of IFN-gamma-induced class II transactivator expression by a 19-kDa lipoprotein from Mycobacterium tuberculosis: a potential mechanism for immune evasion. J Immunol 2003; 171(1):175-84.
121. Noss E H, Harding C V, Boom W H. Mycobacterium tuberculosis inhibits MHC class II antigen processing in murine bone marrow macrophages. Cell Immunol 2000; 201(1):63-74.
122. Pecora N D, Gehring A J, Canaday D H, Boom W H, Harding C V. Mycobacterium tuberculosis LprA is a lipoprotein agonist of TLR2 that regulates innate immunity and APC function. J Immunol 2006; 177(1):422-9.
123. Gehring A J, Dobos K M, Belisle J T, Harding C V, Boom W H. Mycobacterium tuberculosis LprG (Rv1411c): a novel TLR-2 ligand that inhibits human macrophage class II MHC antigen processing. J Immunol 2004; 173(4):2660-8.
124. Hmama Z, Gabathuler R, Jefferies W A, de Jong G, Reiner N E. Attenuation of HLA-DR expression by mononuclear phagocytes infected with Mycobacterium tuberculosis is related to intracellular sequestration of immature class II heterodimers. J Immunol 1998; 161(9):4882-93.
125. Gagliardi M C, Teloni R, Mariotti S, Iona E, Pardini M, Fattorini L, Orefici G, Nisini R. Bacillus Calmette-Guerin shares with virulent Mycobacterium tuberculosis the capacity to subvert monocyte differentiation into dendritic cell: implication for its efficacy as a vaccine preventing tuberculosis. Vaccine 2004; 22(29-30):3848-57.
126. Gagliardi M C, Lemassu A, Teloni R, Mariotti S, Sargentini V, Pardini M, Daffe M, Nisini R. Cell wall-associated alpha-glucan is instrumental for Mycobacterium tuberculosis to block CD1 molecule expression and disable the function of dendritic cell derived from infected monocyte. Cell Microbiol 2007; 9(8):2081-92.
127. Sendide K, Deghmane A E, Pechkovsky D, Av-Gay Y, Talal A, Hmama Z. Mycobacterium bovis BCG attenuates surface expression of mature class II molecules through IL-10-dependent inhibition of cathepsin S. J Immunol 2005; 175(8):5324-32.
128. Engele M, Stossel E, Castiglione K, Schwerdtner N, Wagner M, Bolcskei P, Rollinghoff M, Stenger S. Induction of TNF in human alveolar macrophages as a potential evasion mechanism of virulent Mycobacterium tuberculosis. J Immunol 2002; 168(3):1328-37.
129. Tobian A A, Potter N S, Ramachandra L, Pai R K, Convery M, Boom W H, Harding C V. Alternate class I MHC antigen processing is inhibited by Toll-like receptor signaling pathogen-associated molecular patterns: Mycobacterium tuberculosis 19-kDa lipoprotein, CpG DNA, and lipopolysaccharide. J Immunol 2003; 171(3): 1413-22.
130. Simmons D P, Canaday D H, Liu Y, Li Q, Huang A, Boom W H, Harding C V. Mycobacterium tuberculosis and TLR2 agonists inhibit induction of type I IFN and class I MHC antigen cross processing by TLR9. J Immunol; 185(4):2405-15.
131. Soualhine H, Deghmane A E, Sun J, Mak K, Talal A, Av-Gay Y, Hmama Z. Mycobacterium bovis bacillus Calmette-Guerin secreting active cathepsin S stimulates expression of mature MHC class II molecules and antigen presentation in human macrophages. J Immunol 2007; 179(8):5137-45.
132. Jacobs W R, Jr., Tuckman M, Bloom B R. Introduction of foreign DNA into mycobacteria using a shuttle phasmid. Nature 1987; 327(6122):532-5.
133. Aldovini A, Young R A. Humoral and cell-mediated immune responses to live recombinant BCG-HIV vaccines. Nature 1991; 351 (6326):479-82.
134. Stover C K, de la Cruz V F, Fuerst T R, et al. New use of BCG for recombinant vaccines. Nature 1991; 351 (6326):456-60.
135. da Cruz F W, McBride A J, Conceicao F R, Dale J W, McFadden J, Dellagostin O A. Expression of the B-cell and T-cell epitopes of the rabies virus nucleoprotein in Mycobacterium bovis BCG and induction of an humoral response in mice. Vaccine 2001; 20(5-6):731-6.
136. Bueno S M, Gonzalez P A, Cautivo K M, et al. Protective T cell immunity against respiratory syncytial virus is efficiently induced by recombinant BCG. Proc Natl Acad Sci USA 2008; 105(52):20822-7.
137. Grode L, Kursar M, Fensterle J, Kaufmann S H, Hess J. Cell-mediated immunity induced by recombinant Mycobacterium bovis Bacille Calmette-Guerin strains against an intracellular bacterial pathogen: importance of antigen secretion or membrane-targeted antigen display as lipoprotein for vaccine efficacy. J Immunol 2002; 168(4):1869-76.
138. Abdelhak S, Louzir H, Timm J, et al. Recombinant BCG expressing the leishmania surface antigen Gp63 induces protective immunity against Leishmania major infection in BALB/c mice. Microbiology 1995; 141 (Pt 7):1585-92.
139. Nascimento I P, Dias W O, Quintilio W, Hsu T, Jacobs W R, Jr., Leite L C. Construction of an unmarked recombinant BCG expressing a pertussis antigen by auxotrophic complementation: protection against Bordetella pertussis challenge in neonates. Vaccine 2009; 27(52):7346-51.
140. Barthold S W, Bockenstedt L K. Passive immunizing activity of sera from mice infected with Borrelia burgdorferi. Infect Immun 1993; 61(11):4696-702.
141. Schaible U E, Kramer M D, Eichmann K, Modolell M, Museteanu C, Simon M M. Monoclonal antibodies specific for the outer surface protein A (OspA) of Borrelia burgdorferi prevent Lyme borreliosis in severe combined immunodeficiency (scid) mice. Proc Natl Acad Sci USA 1990; 87(10):3768-72.
142. Stover C K, Bansal G P, Hanson M S, et al. Protective immunity elicited by recombinant bacille Calmette-Guerin (BCG) expressing outer surface protein A (OspA) lipoprotein: a candidate Lyme disease vaccine. J Exp Med 1993; 178(1):197-209.
143. Langermann S, Palaszynski S, Sadziene A, Stover C K, Koenig S. Systemic and mucosal immunity induced by BCG vector expressing outer-surface protein A of Borrelia burgdorferi. Nature 1994; 372(6506):552-5.
144. Stover C K, Bansal G P, Langerman S, Hanson M S. Protective immunity elicited by rBCG vaccines. Dev Biol Stand 1994; 82:163-70.
145. Langermann S, Palaszynski S R, Burlein J E, Koenig S, Hanson M S, Briles D E, Stover C K. Protective humoral response against pneumococcal infection in mice elicited by recombinant bacille Calmette-Guerin vaccines expressing pneumococcal surface protein A. J Exp Med 1994; 180(6):2277-86.
146. Joseph J, Fernandez-Lloris R, Pezzat E, Saubi N, Cardona P J, Mothe B, Gatell J M. Molecular characterization of heterologous HIV-1 gp120 gene expression disruption in mycobacterium bovis BCG host strain: a critical issue for engineering mycobacterial based-vaccine vectors. J Biomed Biotechnol; 2010:357370.
147. Winter N, Lagranderie M, Gangloff S, Leclerc C, Gheorghiu M, Gicquel B. Recombinant BCG strains expressing the SIVmac251 nef gene induce proliferative and CTL responses against nef synthetic peptides in mice. Vaccine 1995; 13(5):471-8.
148. Chujoh Y, Matsuo K, Yoshizaki H, et al. Cross-clade neutralizing antibody production against human immunodeficiency virus type 1 clade E and B′ strains by recombinant Mycobacterium bovis BCG-based candidate vaccine. Vaccine 2001; 20(5-6):797-804.
149. Kawahara M, Matsuo K, Honda M. Intradermal and oral immunization with recombinant Mycobacterium bovis BCG expressing the simian immunodeficiency virus Gag protein induces long-lasting, antigen-specific immune responses in guinea pigs. Clin Immunol 2006; 119(1):67-78.
150. Wiriyarat W, Sukpanichnant S, Sittisombut N, et al. Specific immune response and pathological findings in BALB/c mice inoculated with recombinant BCG expressing HIV-1 antigen. Asian Pac J Allergy Immunol 2005; 23(1):41-51.
151. Someya K, Cecilia D, Ami Y, et al. Vaccination of rhesus macaques with recombinant Mycobacterium bovis bacillus Calmette-Guerin Env V3 elicits neutralizing antibody-mediated protection against simian-human immunodeficiency virus with a homologous but not a heterologous V3 motif. J Virol 2005; 79(3):1452-62.
152. Chege G K, Thomas R, Shephard E G, et al. A prime-boost immunisation regimen using recombinant BCG and Pr55(gag) virus-like particle vaccines based on HIV type 1 subtype C successfully elicits Gag-specific responses in baboons. Vaccine 2009; 27(35):4857-66.
153. Hess J, Miko D, Catic A, Lehmensiek V, Russell D G, Kaufmann S H. Mycobacterium bovis Bacille Calmette-Guerin strains secreting listeriolysin of Listeria monocytogenes. Proc Natl Acad Sci USA 1998; 95(9):5299-304.
154. Grode L, Seiler P, Baumann S, et al. Increased vaccine efficacy against tuberculosis of recombinant Mycobacterium bovis bacille Calmette-Guerin mutants that secrete listeriolysin. J Clin Invest 2005; 115(9):2472-9.
155. Sun R, Skeiky Y A, Izzo A, et al. Novel recombinant BCG expressing perfringolysin O and the over-expression of key immunodominant antigens; pre-clinical characterization, safety and protection against challenge with Mycobacterium tuberculosis. Vaccine 2009; 27(33):4412-23.
156. Sendide K, Deghmane A E, Reyrat J M, Talal A, Hmama Z. Mycobacterium bovis BCG urease attenuates major histocompatibility complex class II trafficking to the macrophage cell surface. Infect Immun 2004; 72(7):4200-9.
157. Mobley H L, Island M D, Hausinger R P. Molecular biology of microbial ureases. Microbiol. Rev 1995; 59(3):451-80.
158. Jones S, Preiter K, Portnoy D A. Conversion of an extracellular cytolysin into a phagosome-specific lysin which supports the growth of an intracellular pathogen. Mol Microbiol 1996; 21(6):1219-25.
159. Rosario M, Hopkins R, Fulkerson J, et al. Novel recombinant Mycobacterium bovis BCG, ovine atadenovirus, and modified vaccinia virus Ankara vaccines combine to induce robust human immunodeficiency virus-specific CD4 and CD8 T-cell responses in rhesus macaques. J Virol; 84(12): δ 898-908.
160. Rosario M, Fulkerson J, Soneji S, et al. Safety and immunogenicity of novel recombinant BCG and modified vaccinia virus Ankara vaccines in neonate rhesus macaques. J Virol; 84(15):7815-21.
161. Rock K L, Rothstein L, Gamble S. Generation of class I MHC-restricted T-T hybridomas. J Immunol 1990; 145(3):804-11.
162. Shen Z, Reznikoff G, Dranoff G, Rock K L. Cloned dendritic cells can present exogenous antigens on both MHC class I and class II molecules. J Immunol 1997; 158(6):2723-30.
163. Kovacsovics-Bankowski M, Rock K L. Presentation of exogenous antigens by macrophages: analysis of major histocompatibility complex class I and II presentation and regulation by cytokines. Eur J Immunol 1994; 24(10):2421-8.
164. Kovacsovics-Bankowski M, Rock K L. A phagosome-to-cytosol pathway for exogenous antigens presented on MHC class I molecules. Science 1995; 267(5195):243-6.
165. Mazzaccaro R J, Gedde M, Jensen E R, van Santen H M, Ploegh H L, Rock K L, Bloom B R. Major histocompatibility class I presentation of soluble antigen facilitated by Mycobacterium tuberculosis infection. Proc Natl Acad Sci USA 1996; 93(21):11786-91.
166. Rubin E J, Akerley B J, Novik V N, Lampe D J, Husson R N, Mekalanos J J. In vivo transposition of mariner-based elements in enteric bacteria and mycobacteria. Proc Natl Acad Sci USA 1999; 96(4):1645-50.
167. Chiang S L, Rubin E J. Construction of a mariner-based transposon for epitope-tagging and genomic targeting. Gene 2002; 296(1-2): 179-85.
168. Bardarov S, Kriakov J, Carriere C, et al. Conditionally replicating mycobacteriophages: a system for transposon delivery to Mycobacterium tuberculosis. Proc Natl Acad Sci USA 1997; 94(20):10961-6.
169. Kriakov J, Lee S, Jacobs W R, Jr. Identification of a regulated alkaline phosphatase, a cell surface-associated lipoprotein, in Mycobacterium smegmatis. J Bacteriol 2003; 185(16):4983-91.
170. Bardarov S, Bardarov Jr S, Jr., Pavelka Jr M S, Jr., et al. Specialized transduction: an efficient method for generating marked and unmarked targeted gene disruptions in Mycobacterium tuberculosis, M. bovis BCG and M. smegmatis. Microbiology 2002; 148(Pt 10):3007-17.
171. Kalpana G V, Bloom B R, Jacobs W R, Jr. Insertional mutagenesis and illegitimate recombination in mycobacteria. Proc Natl Acad Sci USA 1991; 88(12):5433-7.
172. McFadden J. Recombination in mycobacteria. Mol Microbiol 1996; 21(2):205-11.
173. Pelicic V, Reyrat J M, Gicquel B. Expression of the Bacillus subtilis sacB gene confers sucrose sensitivity on mycobacteria. J Bacteriol 1996; 178(4):1197-9.
174. Pavelka M S, Jr., Jacobs W R, Jr. Comparison of the construction of unmarked deletion mutations in Mycobacterium smegmatis, Mycobacterium bovis bacillus Calmette-Guerin, and Mycobacterium tuberculosis H37Rv by allelic exchange. J Bacteriol 1999; 181(16):4780-9.
175. Mercier F, Zervosen A, Teller N, Frere J M, Herman R, Pennartz A, Joris B, Luxen A. 1,6-AnhMurNAc derivatives for assay development of amidase AmiD. Bioorg Med Chem; 18(21):7422-31.
176. Pennartz A, Genereux C, Parquet C, Mengin-Lecreulx D, Joris B. Substrate-induced inactivation of the Escherichia coli AmiD N-acetylmuramoyl-L-alanine amidase highlights a new strategy to inhibit this class of enzyme. Antimicrob Agents Chemother 2009; 53(7):2991-7.
177. Sharma S, Sharma M, Bose M. Mycobacterium tuberculosis infection of human monocyte-derived macrophages leads to apoptosis of T cells. Immunol Cell Biol 2009; 87(3):226-34.
178. Rao V, Gao F, Chen B, Jacobs W R, Jr., Glickman M S. Trans-cyclopropanation of mycolic acids on trehalose dimycolate suppresses Mycobacterium tuberculosis-induced inflammation and virulence. J Clin Invest 2006; 116(6):1660-7.
179. Barkan D, Liu Z, Sacchettini J C, Glickman M S. Mycolic acid cyclopropanation is essential for viability, drug resistance, and cell wall integrity of Mycobacterium tuberculosis. Chem Biol 2009; 16(5):499-509.
180. Ranganathan U D, Larsen M H, Kim J, Porcelli S A, Jacobs W R, Jr., Fennelly G J. Recombinant pro-apoptotic Mycobacterium tuberculosis generates CD8+ T cell responses against human immunodeficiency virus type 1 Env and M. tuberculosis in neonatal mice. Vaccine 2009; 28(1):152-61.
181. Sadagopal S, Braunstein M, Hager C C, et al. Reducing the activity and secretion of microbial antioxidants enhances the immunogenicity of BCG. PLoS One 2009; 4(5):e5531.
182. Im E J, Saubi N, Virgili G, et al. Vaccine platform for prevention of tuberculosis and mother-to-child transmission of human immunodeficiency virus type 1 through breastfeeding. J Virol 2007; 81(17):9408-18.
183. Sassetti C M, Boyd D H, Rubin E J. Genes required for mycobacterial growth defined by high density mutagenesis. Mol Microbiol 2003; 48(1):77-84.
184. Abdallah A M, Verboom T, Hannes F, et al. A specific secretion system mediates PPE41 transport in pathogenic mycobacteria. Mol Microbiol 2006; 62(3):667-79.
185. Tundup S, Pathak N, Ramanadham M, Mukhopadhyay S, Murthy K J, Ehtesham N Z, Hasnain S E. The co-operonic PE25/PPE41 protein complex of Mycobacterium tuberculosis elicits increased humoral and cell mediated immune response. PLoS One 2008; 3(10):e3586.
186. Buchmeier N, Blanc-Potard A, Ehrt S, Piddington D, Riley L, Groisman E A. A parallel intraphagosomal survival strategy shared by mycobacterium tuberculosis and Salmonella enterica. Mol Microbiol 2000; 35(6): 1375-82.
187. Ramage H R, Connolly L E, Cox J S. Comprehensive functional analysis of Mycobacterium tuberculosis toxin-antitoxin systems: implications for pathogenesis, stress responses, and evolution. PLoS Genet. 2009; 5(12):e1000767.
188. Klumpp J, Fuchs T M. Identification of novel genes in genomic islands that contribute to Salmonella typhimurium replication in macrophages. Microbiology 2007; 153(Pt 4):1207-20.
189. George K M, Yuan Y, Sherman D R, Barry C E, 3rd. The biosynthesis of cyclopropanated mycolic acids in Mycobacterium tuberculosis. Identification and functional analysis of CMAS-2. J Biol Chem 1995; 270(45):27292-8.
190. Bhatt A, Fujiwara N, Bhatt K, et al. Deletion of kasB in Mycobacterium tuberculosis causes loss of acid-fastness and subclinical latent tuberculosis in immunocompetent mice. Proc Natl Acad Sci USA 2007; 104(12):5157-62.
191. Yuan Y, Lee R E, Besra G S, Belisle J T, Barry C E, 3rd. Identification of a gene involved in the biosynthesis of cyclopropanated mycolic acids in Mycobacterium tuberculosis. Proc Natl Acad Sci USA 1995; 92(14):6630-4.
192. Barkan D, Rao V, Sukenick G D, Glickman M S. Redundant function of cmaA2 and mmaA2 in Mycobacterium tuberculosis cis cyclopropanation of oxygenated mycolates. J Bacteriol; 192(14):3661-8.
193. Geisel R E, Sakamoto K, Russell D G, Rhoades E R. In vivo activity of released cell wall lipids of Mycobacterium bovis bacillus Calmette-Guerin is due principally to trehalose mycolates. J Immunol 2005; 174(8):5007-15.
194. Iyer L M, Abhiman S, Maxwell Burroughs A, Aravind L. Amidoligases with ATP-grasp, glutamine synthetase-like and acetyltransferase-like domains: synthesis of novel metabolites and peptide modifications of proteins. Mol Biosyst 2009; 5(12):1636-60.
195. Burns K E, Darwin K H. Pupylation: A Signal for Proteasomal Degradation in Mycobacterium tuberculosis. Subcell Biochem; 54:149-57.
196. Herr H W, Morales A. History of bacillus Calmette-Guerin and bladder cancer: an immunotherapy success story. J Urol 2008; 179(1):53-6.
197. Brandau S, Suttmann H. Thirty years of BCG immunotherapy for non-muscle invasive bladder cancer: a success story with room for improvement. Biomed Pharmacother 2007; 61(6):299-305.

1. A mycobacterium comprising one or more mutations that ablate or reduce expression of at least one gene selected from BCG_{—}0381, BCG_{—}0546c, BCG_{—}0992, BCG_{—}0993, BCG_{—}1472c, BCG_{—}1790, BCG_{—}1964, BCG_{—}2067c, BCG_{—}2384c, BCG_{—}2449c, BCG_{—}2580, BCG_{—}2588, BCG_{—}2589, BCG_{—}3231C, BCG_{—}3297, BCG_{—}3445, and BCG_{—}3808c, or a homolog thereof, or an operon that includes said gene.

2. The mycobacterium of claim 1, wherein said mutation is a deletion, substitution, or insertion.

3. The mycobacterium of claim 2, wherein said mutation is a deletion of all or a part of said gene.

4. The mycobacterium of claim 1, wherein mycobacterium is selected from M. africanum, M. avium, M. bovis, M. canetti, M. chelonae, M. fortuitum, M. gordonae, M. hiberniae, M. intracellulare, M. leprae, M. kansasii, M. marinum, M. microti, M. paratuberculosis, M. phlei, M. pinnipedii, M. scrofulaceum, M. simiae, M. smegmatis, M. szulgai, M. tuberculosis, M. ulcerans, M. vacca, and M. xenopi.

5. The mycobacterium of claim 4, wherein said mycobacterium is M. bovis BCG.

6. The mycobacterium of claim 1 engineered to express a foreign antigen.

7. The mycobacterium of claim 6, wherein said foreign antigen is from a pathogen, a cancer cell, an allergen, is associated with an autoimmune disease or graft rejection, or is a cytokine, a chemokine, an immunoregulatory agent, or a therapeutic agent.

8. The mycobacterium of claim 7, wherein said pathogen is a virus, bacteria, fungus, or parasite.

9.-12. (canceled)

13. The mycobacterium of claim 6, wherein said antigen is an HIV antigen selected from the group consisting of gpl20 env, gpl40 env, gp160 env, gag, pol, vif, vpr, vpu, tat, rev and nef.

14.-15. (canceled)

16. The mycobacterium of claim 13, wherein said mycobacterium is formulated as a vaccine.

17. (canceled)

18. The mycobacterium of claim 1, wherein said mycobacterium is clone G9, J13, A79, AK27, C46, K14, BC15, CN11, A25, AF30, C63, C57, AF25, BL2, AE29, AZ11, or CX18.

19. (canceled)

20. An antigenic composition comprising the mycobacterium of claim 6 and a pharmaceutically acceptable carrier, diluent, and/or excipient and, optionally, an adjuvant.

21.-23. (canceled)

24. A method of inducing an immune response in a mammal against an antigen comprising administering the antigenic composition of claim 20 to said mammal.

25.-30. (canceled)

31. The method of claim 24, wherein said mammal is a human.

32. The method of claim 24, wherein said composition is administered as either a priming composition or a boosting composition in a prime-boost immunization.

33. The method of claim 32, wherein said composition is administered as the priming composition and said boosting composition is selected from a recombinant adenovirus vector (rAd) expressing an antigen, epitope, and/or polypeptide for which an immune response is sought to be generated, a NYVAC vector expressing an antigen, epitope, and/or polypeptide against which an immune response is sought to be generated, and a composition that includes an antigen, epitope, and/or polypeptide (e.g., a protein antigen) against which an immune response is sought to be generated.

34. A kit for immunizing a mammal against a disease or disorder comprising the antigenic composition of claim 20 and instructions for use.

35. A method of optimizing a mycobacterial vaccine vector comprising:
a) mutagenizing a parental mycobacterial strain expressing an antigen by disruption of one or more genes of said parental mycobacterial strain using a transposon to produce a mutated mycobacterium; and
b) assaying said mutated mycobacterium for MHC class I presentation of said antigen, wherein an increase in MHC class I presentation of said antigen relative to said parental mycobacterial strain indicates said mutated mycobacterium is an optimized mycobacterial vaccine vector.

36.-41. (canceled)

42. A method of inducing an immune response in a mammal against an antigen comprising administering the mycobacterium of claim 1 to said mammal.
